(19) United States
(12) Patent Application Publication
(10) Pub. No.: US 2024/0368229 A1
(43) Pub. Date: Nov. 7, 2024

(54) Cell-Penetrating Peptides and Methods of Use Thereof

(71) Applicant: YALE UNIVERSITY, New Haven, CT (US)

(72) Inventors: Daniel DiMaio, New Haven, CT (US); Pengwei Zhang, New Haven, CT (US); Gabriel Monteiro Da Silva, Ribeirao Preto (BR); Weiya Bai, Shanghai (CN)

(21) Appl. No.: 18/757,397

(22) Filed: Jun. 27, 2024

Related U.S. Application Data
(63) Continuation of application No. 17/057,501, filed on Nov. 20, 2020, now Pat. No. 12,060,388.
(60) Provisional application No. 62/676,706, filed on May 25, 2018.

(51) Int. Cl. C07K 14/005; A61K 9/00; A61K 38/00; A61P 31/20; C12N 7/00

(52) U.S. Cl. CPC C07K 14/005; A61K 9/0014; A61P 31/20; C12N 7/00; A61K 38/00; C07K 2319/33; C12N 2710/20022; C12N 2710/20033

(57) ABSTRACT

In various aspects and embodiments the invention provides compositions and methods for facilitating cell penetration of a cargo molecule. In another aspect, the invention provides a method of preventing viral infection in a subject in need thereof, the method comprising providing to the subject a therapeutically effective amount of a polypeptide comprising a cell-penetrating peptide and a retromer binding site.

### CROSS REFERENCE TO RELATED APPLICATIONS

The present application claims priority under 35 U.S.C. § 119(e) to U.S. Provisional Patent Application No. 62/676,706, filed May 25, 2018, which is incorporated herein by reference in its entirety.

### STATEMENT REGARDING FEDERALLY SPONSORED RESEARCH OR DEVELOPMENT

This invention was made with government support under CA016038 and AI102876 awarded by National Institutes of Health. The government has certain rights in the invention.

### SEQUENCE LISTING

The instant application contains a Sequence Listing which has been submitted in ASCII format via Patent Center and is hereby incorporated by reference in its entirety. Said ASCII copy, created on Jul. 27, 2024, is named “047162-7170US2_Sequence Listing,” and is 705,600 bytes in size.

### BACKGROUND OF THE INVENTION

Cell-penetrating peptides as vectors for cell entry show great promise as research tools or for drug delivery. However, the majority of cell-penetrating peptides and thus the associated cargo molecules are sequestered in endosomes and eventually degraded.

There is a need in the art for cell-penetrating peptides that allow for transport of attached cargo into the cell, and yet avoid endosome sequestration and consequent degradation of the cargo. The present disclosure addresses this need.

### SUMMARY OF THE INVENTION

In one aspect, the invention provides a method of delivering a cargo molecule to the cytoplasm of at least one cell, the method comprising contacting the cell with a transport construct comprising the cargo molecule and at least one cell-penetrating peptide selected from the group consisting of SEQ ID NOs:1-164, or a salt or solvate thereof.

In another aspect, the invention provides a method of promoting endosomal escape for a cargo molecule, the method comprising contacting at least one cell with a transport construct comprising the cargo molecule and at least one cell-penetrating peptide selected from the group consisting of SEQ ID NOs:1-164, or a salt or solvate thereof.

In various embodiments, the cell-penetrating peptide is SEQ ID NO: 2.

In various embodiments, the transport construct further comprises at least one selected from the group consisting of an activity modulating flanking sequence, a temperature modulating flanking sequence, and a pH modulating flanking sequence.

In various embodiments, the transport construct further comprises a linker connecting the cargo molecule and the cell-penetrating peptide.

In various embodiments, the cargo molecule is at least one selected from the group consisting of a nucleic acid; peptide; protein; peptide-nucleic acid; oligosaccharide; lipid; glycolipid; lipoprotein; small molecule compound; therapeutic drug; UV-vis, fluorescent or radioactive label; imaging agent; diagnostic agent; prophylactic agent; liposome; and virus.

In various embodiments, the at least one cell is human.

In various embodiments, the at least one cell is in a human subject.

In various embodiments, the contacting comprises administering a pharmaceutical composition comprising an effective amount of the transport construct and at least one pharmaceutically acceptable carrier to the human subject.

In another aspect, the invention provides an isolated transport construct comprising a cargo molecule and at least one cell-penetrating peptide selected from the group consisting of SEQ ID NOs: 165-517, or a salt or solvate thereof.

In various embodiments, the cell-penetrating peptide is SEQ ID NO: 2.

In various embodiments, the transport construct further comprises at least one selected from the group consisting of an activity modulating flanking sequence, a temperature modulating flanking sequence, and a pH modulating flanking sequence.

In various embodiments, the transport construct further comprises a linker connecting the cargo molecule and the cell-penetrating peptide.

In various embodiments, the cargo molecule is at least one selected from the group consisting of a nucleic acid; peptide; protein; peptide nucleic acid; oligosaccharide; lipid; glycolipid; lipoprotein; small molecule compound; therapeutic drug; UV-vis, fluorescent or radioactive label; imaging agent; diagnostic agent; prophylactic agent; liposome; and virus.

In various embodiments, the invention provides a pharmaceutical composition comprising the transport construct and at least one pharmaceutically acceptable excipient.

In another aspect, the invention provides a method of preventing viral infection in a subject in need thereof, the method comprising providing to the subject a therapeutically effective amount of a polypeptide comprising a cell-penetrating peptide and a retromer binding site.

In various embodiments, the viral infection is papillomavirus, hepatitis C virus, influenza virus or human immunodeficiency virus (HIV).

In various embodiments, the therapeutically effective amount of the polypeptide is formulated for topical administration.

In various embodiments, the polypeptide comprises at least one cell-penetrating peptide selected from the group consisting of SEQ ID NOS: 165-517.

In various embodiments, the polypeptide comprises a retromer binding site comprising the sequence FYL.

### BRIEF DESCRIPTION OF THE DRAWINGS

The following detailed description of preferred embodiments of the invention will be better understood when read in conjunction with the appended drawings. For the purpose of illustrating the invention, certain embodiments are shown in the drawings. It should be understood, however, that the invention is not limited to the precise arrangements and instrumentalities of the embodiments shown in the drawings.

FIGS. 1A-1F show that the basic region of human papillomavirus (HPV) 16 L2 can be replaced by a cationic cell-penetrating motif FIG. 1A depicts the amino acid sequence of the C-terminus of the L2 protein of various HPV types. Basic amino acids downstream of the FYL major retromer binding motif are shown. Numbers indicate the amino acid position in the 473-residue HPV16 L2 protein. The membrane-destabilizing sequence in HPV33 L2 is underlined. FIG. 1B depicts the sequence of the C-terminus of wild-type and mutant HPV16 L2 proteins. In FIGS. 1C-1D HeLa S3 cells were infected with wild-type (WT) or mutant HPV16 pseudovirus (PsV) stocks containing equal numbers of the HcRed reporter plasmid (corresponding to multiplicity of infection (MOI) of one for wild-type). Forty-eight hours post-infection (h.p.i.), infectivity was measured by using flow cytometry to determine the fraction of fluorescent cells. The results were normalized to the fraction of cells infected by wild-type. The mean results and standard deviation of three or more independent experiments for each sample are shown. *p<0.05; **p<0.01. In FIG. 1E, HeLa S3 cells were transfected with a RISC-free control siRNA (black bars) or siRNA targeting retromer subunit Vps29 (grey bars), followed by infection at MOI of one with wild-type or L2-Tat HPV16 PsV. Infectivity was measured (n=3) and displayed as in FIG. 1C. In FIG. 1F HeLa S3 cells were left untreated (black bars) or pretreated with 250 nM γ-secretase inhibitor XXI for one h at 37° C. (grey bars), and then infected at MOI of one with wild-type or L2-Tat HPV16 PsV. Infectivity was measured (n=3) and displayed as described in FIG. 1C. See also FIGS. 7A-7B and Table 1.

FIGS. 2A-2D show that the L2 basic sequence displays cell-penetrating activity. FIG. 2A depicts the amino acid sequence of HPV16 L2 C-terminal peptides containing the wild-type (WT) basic sequence or the six alanine (6A) or three arginine (3R) mutations conjugated to Alexa Fluor 488. 293T cells were mock-treated or incubated with fluorescent peptides for three h at 37° C. and examined by confocal microscopy. FIG. 2B: Schematic diagram and C-terminal sequences of GFP-L2 fusion proteins containing the wild-type or mutant L2 C-terminal sequence. FIG. 2C: HaCaT and HeLa S3 cells were incubated with GFP-L2 fusion proteins for five h. Cells were examined by confocal microscopy, then treated for 10-15 min with 0.04% trypan blue (+TB) to quench extracellular fluorescence, and the same fields were re-imaged. FIG. 2D: HeLa S3 and HaCaT cells were incubated with GFP-L2 fusion proteins for various times. Cells were then treated with trypsin, and fluorescence was measured by flow cytometry. Mean fluorescent intensity (MFI) was plotted at the indicated time periods.

FIGS. 3A-3F show that the basic region of HPV16 L2 is not essential for virus binding and internalization. FIG. 3A: HeLa-sen2 cells were mock-infected or incubated at 4° C. for two h at MOI of 20 with wild-type, 6A, or 3R HPV16 PsV. Non-permeabilized cells were stained with anti-L1 antibody (green) and examined by confocal microscopy. FIG. 3B: HeLa-sen2 cells were treated as described in FIG. 3A, detached with 0.5 mM EDTA for 10 min, and stained with rabbit polyclonal anti-L1 antibody. MFI of the cells was measured by flow cytometry and normalized to cells incubated with wild-type HPV16 PsV. Mean results and standard deviation are shown (n=3). ***p<0.001; ns, not significant. FIG. 3C: HeLa cells were treated as described in FIG. 3A. After two h, cells were washed, lysed, and bound virus was assessed by SDS-polyacrylamide gel electrophoresis and blotting for L1 (top panel). GAPDH was used as a loading control (bottom panel). FIG. 3D: HeLa-sen2 cells were treated and processed as described in FIG. 3C with PsV containing wild-type or 6A mutant L2 or with L1-only PsV lacking L2. FIG. 3E: HeLa cells-sen2 were mock-infected or infected at MOI of 20 at 4° C. for two h with wild-type, 6A or 3R HPV16 PsV and then washed and shifted to 37° C. for six or 16 h to allow internalization. Permeabilized cells were stained with anti-L1 antibody (green) and examined by confocal microscopy. FIG. 3F: HeLa-sen2 cells were infected as described in FIG. 3E, and 6 h after shift to 37° C. harvested by trypsinization. Permeabilized cells were stained with anti-L1 antibody and subjected to flow cytometry. MFI of the cell populations was normalized to cells infected with wild-type HPV16 PsV. Mean results and standard deviation are shown (n=3). *p<0.05. See also FIG. 11B.

FIGS. 4A-4B show that the 3R mutant PsV accumulates in endosomes and does not reach trans-Golgi network (TGN). FIG. 4A: HeLa-sen2 cells were mock-infected or infected at 37° C. at MOI of 100 with wild-type, 3R or retromer binding site (DM) mutant HPV16 PsV. At eight and 16 h.p.i., PLA was performed with anti-L1 antibody and either an EEA1 (FIG. 4A) or a TGN46 (FIG. 4B) antibody. PLA signal is green. Multiple images obtained as in left panels were processed with BlobFinder software to determine the fluorescence intensity per cell. The graphs in the right panels show mean results and standard deviation (n=3), in which the results for the EEA1/L1 samples and the TGN46/L1 samples were normalized to those of cells infected with wild-type HPV16 PsV at eight and 16 h.p.i., respectively. Black bars, 8 h.p.i.; grey bars, 16 h.p.i. **p<0.01; ***p<0.001.

FIGS. 5A-5D show that the 3R mutant is defective in accessing retromer during HPV infection. FIG. 5A: HeLa-sen2 cells were infected as in FIG. 4A. At eight and 16 h.p.i., PLA was performed with anti-L1 antibody and an antibody recognizing Vps35 to assess L1 in proximity to retromer. FIG. 5B: Multiple images obtained as in FIG. 5A were processed by using BlobFinder software to determine the fluorescence intensity per cell. The graph shows the mean intensity and standard deviation for the mutant samples normalized to that of cells infected with wild-type PsV at eight h.p.i. Black bars, wild-type; dark grey bars, 3R mutant; light grey bars, DM mutant. *p<0.05; **p<0.01; ***p<0.001. FIG. 5C: Retromer adsorbed to glutathione beads via GST-tagged Vps35 was incubated with purified GFP-L2 fusion proteins. After pull-down, proteins bound to retromer were separated by SDS-PAGE and detected by blotting with anti-GFP antibody (top panel). Bottom panel shows input GFP-L2 fusion proteins. FIG. 5D: Top portion shows sequences of wild-type and mutant biotinylated peptides from the C-terminus of L2. Bottom portion. Peptides were incubated with extracts of uninfected HeLa S3 cells, pulled-down with streptavidin, and separated by SDS-PAGE. Retromer associated with the peptide was detected by blotting for Vps35.

FIGS. 6A-6D show reconstitution of split GFP by infection with PsV containing L2-GFP11. FIG. 6A: Schematic diagrams of wild-type L2 protein and derivatives with GFP11 (green) inserted upstream of RKRRKR (L2-GFP11-CPP) or at the C-terminus of L2 (L2-CPP-GFP11). The bulk of the L2 protein is shown in blue; wild-type and mutant CPP in red; and retromer binding sites in purple. See FIGS. 8A-8F for sequences. FIG. 6B: Clonal HaCaT/GFP1-10NES cells were infected at MOI of 2000 with untagged HPV16 PsV or with a PsV containing GFP11-tagged L2. Three h.p.i., cells were examined by confocal microscopy for GFP fluorescence. FIG. 6C: Fluorescence of cells in images as in FIG. 6B was quantified for ˜250 cells for each condition. Results show the fluorescence intensity of individual cells from three independent experiments. ***p<0.0001. FIG. 6D: Clonal HaCaT/GFP1-10NES cells were infected at MOI of 2000 with HPV16 or PsV containing L2-GFP 11-CPP or L2-CPP-GFP 11 with the wild-type L2 or the 3R mutation. Three h.p.i., cells were examined by confocal microscopy for GFP fluorescence. See FIGS. 8A-12B.

FIGS. 7A-7C show the assembly and infectivity of L2 CPP mutants. HPV16 PsV containing wild-type (WT) L2 or the six alanine (6A) or three arginine (3R) mutants were examined by (FIG. 7A) transmission electron microscopy or (FIG. 7B) SDS-PAGE and staining with coomassie blue. FIG. 7C: HeLa S3 (black bars) and HaCaT (grey bars) cells were infected with wild-type, 6A, or 3R mutant HPV16 PsV at MOI of one. Two days later, infection was measured by flow cytometry for HcRed expression and normalized to infection by wild-type. Average results ±standard deviation are shown (n=3). Size bars in FIG. 7A are 50 nm. Numbers in FIG. 7B show molecular mass (in kDa) of size markers.

FIGS. 8A-8F depict the validation of the split GFP assay. (FIG. 8A) Schematic diagram of GFP1-10 construct with a C-terminal nuclear export signal (blue). (FIG. 8B) Parental HaCaT cells and HaCaT cells expressing GFP1-10NES were analyzed by immunostaining with anti-GFP antibody (green) and confocal microscopy. (FIG. 8C) Schematic diagram of CD8-CIMPR and calnexin (CNX) fusion proteins used to validate the split GFP system. All proteins contain a transmembrane domain (TM). The GFP11 segment is in the luminal domain of GFP11-CD8-CIMPR and the cytoplasmic domain of the CD8-CIMPR-GFP11. Both proteins contain a WLM-to-AAA mutation that inactivates the retromer binding site in the cytoplasmic segment. The GFP1-10 segment is in the luminal domain of GFP1-10-CXN and the cytoplasmic domain of CXN-GFP1-10. (FIG. 8D) Cartoon showing cytoplasmic localization of the GFP11 segment in CD8-CIMPR-GFP11 reconstituting GFP fluorescence in HaCaT/GFP1-10NES cells, and luminal localization of the GFP11 segment in GFP11-CD8-CIMPR, reconstituting GFP fluorescence in cells expressing GFP1-10-CNX. (FIG. 8E) A plasmid expressing GFP11-CD8-CIMPR or CD8-CIMPR-GFP11 was transfected into clonal HaCaT/GFP1-10NES cells. Reconstituted GFP fluorescence is shown. (FIG. 8F) 293T cells were co-transfected with a plasmid expressing GFP11-CD8-CIMPR and a plasmid expressing GFP1-10 fused to the N-terminal luminal domain or the C-terminal cytoplasmic domain of calnexin (i.e., GFP1-10-CNX or CXN-GFP1-10, respectively). Reconstituted GFP fluorescence is shown.

FIGS. 9A-9B depict infection by GFP11-tagged HPV16 pseudovirus. FIG. 9A depicts the amino acid sequence of the C-terminus of wild-type and mutant HPV16 L2 proteins with inserted copies of GFP11. FIG. 9B depicts HeLa S3 cells were infected at MOI (determined by the number of encapsidated reporter genomes) of one with the indicated HPV16 PsV containing an hcRed reporter gene. After 48 h, the fraction of fluorescent cells was determined by flow cytometry and normalized to cells infected with wild-type PsV. Mean results ±standard deviation are shown (n=3).

FIG. 10 depicts early reconstitution of GFP fluorescence in infected cells. Clonal HaCaT/GFP1-10NES cells were infected at MOI of 2000 with untagged HPV16 PsV (WT) or a PsV containing GFP11-tagged L2. Cells were examined by confocal microscopy for GFP fluorescence (pseudo-colored black) at three h.p.i. Graph shows quantitation of fluorescent signal at 1.5 (black bars) and three (grey bars) h.p.i. normalized to cells infected with HPV16 PsV containing untagged L2 at 1.5 h.p.i.

FIGS. 11A-11B depict the rapid internalization of HPV16 PsV. FIG. 11A depicts HaCaT cells mock-infected or infected at MOI of 200 with wild-type HPV16 PsV or GFP11-tagged PsV with a wild-type or 3R mutant CPP. Three h.p.i., cells were stained with mouse polyclonal anti-L1 antibody (BD Sciences, #554171) and examined by confocal microscopy. FIG. 11B: HaCaT cells were mock-infected or infected at an MOI of 200 with wild-type HPV16 PsV. Cells were stained with conformation-specific 33L1-7 antibody at the indicated times and examined by confocal microscopy.

FIGS. 12A-12B show that cell cycle progression is required for HPV infection but not L2 protrusion. FIG. 12A depicts HeLa S3 cells left untreated or treated with 6 μM aphidicolin. Twenty-four h later, cells were infected at MOI of one with wild-type HPV16 PsV or PsV containing L2-GFP11-CPP or L2-CPP-GFP11. Forty-eight h.p.i., the fraction of infected cells was determined by flow cytometry for HcRed fluorescence and normalized to untreated cells infected with wild-type HPV16 PsV. FIG. 12B depicts HaCaT/GFP1-10NES cells infected at an MOI of 2000 with wild-type HPV16 PsV or PsV containing GFP11-tagged L2 in the presence of absence of 6 μM aphidicolin. Three h.p.i., cells were examined by confocal microscopy for reconstituted GFP fluorescence. Fluorescence in individual cells (˜250 for each condition) is plotted from two independent experiments. There were no statistically significant differences among samples infected with either GFP11-tagged PsV in the presence or absence of aphidocolin.

FIG. 13 shows that GFP fusion protein containing the HPV16 L2 CPP alone is active in cell binding and internalization, but is not as efficient at cell binding and internalization as a GFP fusion protein containing the HPV16 L2 CPP plus flanking upstream L2 sequences. HaCaT cells were incubated for three hours at 37° C. at pH 7 with the indicated concentration of GFP fusion protein containing only the HPV16 L2 CPP or the L2 CPP plus 21 flanking upstream amino acids from L2. The cells were then imaged in the presence or absence of trypan blue. Trypan blue quenches extracellular fluorescence. In the sequences of peptides below the images, the HPV16 L2 CPP (RKRRKR), SEQ ID NO: 2, and the retromer binding site (FYL) are shown in bold.

FIG. 14 shows that GFP fusion protein with full-length HPV16 C-terminus (HPV16 L2) binds and enters HaCaT cells poorly at pH 7 but well at pH 4. A GFP fusion protein lacking the C-terminal 12 amino acids of L2 (GFP-21P-CPP) binds at pH 7. HaCaT cells were incubated in the absence of GFP fusion protein (mock) or with the indicated GFP fusion protein for three hours at 37° C. at pH 7 or pH 4. The cells were then imaged in the presence or absence of trypan blue (TB). GFP-HPV16 L2 contains 12 amino acids from the end of the basic CPP to the C-terminus of the native protein. These 12 amino acids are not present in GFP-21P-CPP.

FIG. 15 shows that GFP fusion protein with HPV5 C-terminus binds HaCaT cells better at 30° C. than at 37° C. HaCaT cells were incubated for three hours at pH 7 or pH 4 at 30° C. or 37° C. in the absence of GFP fusion protein (mock) or with the GFP fusion protein containing the C-terminus of HPV5. HPV5 is a papillomavirus that infects skin, which is at a lower temperature than core body temperature of 37° C.

FIG. 16 shows that GFP 11 fused to truncated HPV16 C-terminus reconstitutes cytoplasmic fluorescence in HaCaT/GFP1-10NES cells. HaCaT/GFP1-10NES cells were incubated for three hours at pH 7 at 37° C. in the absence of GFP fusion protein or with a short fusion protein consisting of seven tandem copies of GFP11 fused to the C-terminus of L2 terminating at the L2 CPP. The cells were then imaged in the presence or absence of trypan blue (TB). Reconstituted fluorescence is resistant to trypan blue, indicating the delivery of the C-terminus of L2 into the cytoplasm.

FIG. 17 shows that the HPV16 L2 CPP can deliver a peptide into cells that inhibits HPV16 infection. HeLa cells were treated at 37° C. with 4 μg L2-C, a peptide containing the HPV16 L2 retromer binding site and CPP or mock treated. B represents N terminal biotinylation and Am represents C-terminal amidation. Two hours later, the cells were infected for two hours at 37° C. with wild-type HPV16 pseudovirus. Cells were washed and medium was replenished with medium containing the peptide for 24 hours. Infectivity was tested 48 hours later by using flow cytometry to measure fluorescence of HcRed expressed from a plasmid encapsidated in the pseudovirus particle. Similar results were obtained in two independent experiments.

FIGS. 18A-18E depict the identification of peptides that inhibit HPV infection. FIG. 18A (top) depicts sequences of L2 peptides. The top line shows wild-type P16/16 peptide with CPP segment and retromer binding site (RBS) highlighted. Amino acid substitutions in the CPP or RBS mutant peptides are shown in orange and blue, respectively. Bottom: RBS and flanking sequences of indicated HPV L2 proteins or DMT1-II. FIG. 18B depicts an inhibitory dose-response curve for P16/16 peptide. 5×10⁴ HeLa-S3 in 24-well plates were pretreated with various concentrations of P16/16 for one hour prior to infection with HPV pseudovirus (PsV) at multiplicity of infection (MOI) of 1. Peptide was left in the medium for the duration of the experiment. Cells were assessed by flow cytometry to determine the fraction of cells expressing reporter protein HcRed at 48 hours post-infection (h.p.i.). Graph shows average results of three independent experiments, +/−standard deviation. FIG. 18C depicts 5×10⁴ HeLa-S3 in 24-well plates were pretreated with 14 μM of the indicated peptide for one hour, followed by infection with HPV PsV at MOI of one. Peptides were left in the medium for the duration of the experiment. As a control, cells were incubated with the solution used to dissolve peptide. Forty-eight h.p.i., cells were assessed by flow cytometry to determine fraction of cells expressing reporter protein HcRed. Graph shows average results of three independent experiments, +/−standard deviation, normalized to infection of each PsV type in the absence of peptide. P16/16, P16/Tat, and P16/31 caused statistically significant (p<0.01) inhibition of all three PsV types. For the comparison of P16/16 to P16/6A, P16/3R, and PDM/16 for HPV16 PsV, **, p<0.01; ***, p<0.001. Similar levels of significance were achieved with these peptides and HPV18 and HPV5 PsV. FIG. 18D depicts inhibition of HPV infection of HaCaT cells. HaCaT keratinocytes were infected at MOI of 1 with HPV16 PsV in the presence and absence of 14 μM P16/16, and infectivity was assessed 48 h.p.i. by flow cytometry for HcRed fluorescence. FIG. 18E depicts inhibition of authentic HPV16. HeLa cells were infected with HPV16 harvested from organotypic cultures of human keratinocytes or with HPV16 PsV in the presence (grey bars) or absence (black bars) of P16/16. Successful infection by HPV16 and HPV16 PsV was assessed by quantitative reverse transcription-PCR (qRT-PCR) for expression of HPV early region and HcRed mRNA, respectively, and normalized to infection by the cognate virus in the absence of peptide. Background signal determined with non-cognate primers was <0.01%.

FIGS. 19A-19B show that peptide binds retromer and blocks retromer association with HPV. FIG. 19A depicts uninfected HeLa cells were incubated with 14 μM bP16/16 or bPDM/16 for three hours (“b” refers to peptides with an N-terminal biotin moiety). Cells were then fixed, permeabilized, and stained with AlexaFluor-streptavidin (green) and with anti-VPS35 (red). Overlapping signal is pseudocolored yellow. Nuclei were stained blue with DAPI. Mander's correlation coefficients for overlap between streptavidin and VPS35 staining are shown at right, with each spot representing an individual cell. Approximately 100 cells were analyzed for each condition. FIG. 19B depicts 5×10⁴ HeLa-S3 cells grown on glass coverslips for 16 hours were incubated for one hour with or without 14 μM P16/16 or PDM/16, followed by mock-infection or infection with HPV16 PsV at MOI of 200. At 8 or 16 (or both) h.p.i., cells were fixed and processed for PLA with anti-L1 antibody and an antibody recognizing retromer subunit VPS35. PLA signal is green and the nuclei are stained blue with DAPI. Images were acquired by a Leica SP5 confocal microscope. Approximately 200 cells in each sample were imaged. Images show results of a representative experiment. Images were processed by Fiji and quantitatively analyzed by BlobFinder software to measure total fluorescence intensity per cell in each sample. The PLA signal was normalized to that of cells infected with wild-type HPV16 PsV in the absence of peptide at 8 h.p.i. Graph shows the average of total fluorescence per cell, +/−standard deviation, from three independent experiments. n.s., not significant; **, p<0.01; ***p<0.001.

FIGS. 20A and 20B show that the peptide inhibits HPV exit from endosome. HeLa-S3 cells grown on glass coverslips for 16 hours were incubated for one hour with or without 14 μM P16/16, followed by mock-infection or infection with HPV16 PsV at MOI of 200. 8 and 16 h.p.i., cells were processed for PLA with anti-L1 antibody and an antibody recognizing EEA1 (FIG. 20A) or TGN46 (FIG. 20B). PLA was performed as in FIG. 19B. The PLA signal for EEA1/L1 was normalized to that of cells infected with HPV16 PsV in the absence of peptide at 8 h.p.i., while the TGN46/L1 signal was normalized to untreated cells at 16 h.p.i.

FIG. 21 shows that the L2 peptide does not display toxicity. HeLa-S3 cells were incubated with medium, medium plus vehicle, or medium plus 14 μM P16/16 for 4 or 24 hours. Viability assay was performed by using the CellTiter-Fluor viability assay protocol (Promega, Cat #G6082). Briefly, an equal volume of CellTiter-Fluor reagent was added to each well without removing the medium, mixed briefly, then incubated for one hour at 37° C. Fluorescence was measured by using a GloMax Explorer Multimode Microplate Reader (380-400 nmEx/505 nmEm). n.s., not significant.

FIG. 22A-22C show the timing of L2 peptide inhibition. FIG. 22A depicts HeLa cells in 24-well plates infected with HPV16 PsV at MOI of 1. 14 μM P16/16 peptide was added at the time of infection (0) or at the indicated h.p.i., and cells were analyzed by flow cytometry 48 h.p.i. to determine reporter protein fluorescence as a measure of infection. FIG. 22B depicts HeLa-S3 in 24-well plates were incubated with 14 μM P16/16 or vehicle for one hour, followed by infection with HPV16 PsV at MOI of 1. Peptide was left in the medium for the duration of the experiment. Cells were analyzed by flow cytometry 48, 72, or 96 h.p.i. to determine fluorescence. Infectivity was normalized to that in the absence of peptide at each timepoint. ***, p<0.0005. FIG. 22C depicts HeLa cells mock-infected (line A) or infected with HPV16 PsV at MOI of 1 in the absence of peptide (blue, Line B) or in the presence of P16/16. Virus was removed from all infected samples after 24 h. At this time, peptide was added back to some samples (Line C) or removed (Line D). Cells were harvested at various times and subjected to flow cytometry to measure HcRed fluorescence. Panels show histograms at the indicated h.p.i.

FIG. 23 shows that L2 peptide does not inhibit SV40 infection. HeLa-S3 cells were treated with 14 μM P16/16 peptide or vehicle for one hour then infected with SV40 at MOI of ˜1. Forty-eight h.p.i., cells were washed, fixed, and permeabilized with cold methanol for 30 min. Cells were stained for intracellular SV40 large T antigen with fluorophore-conjugated monoclonal antibody PAb101, followed by flow cytometry. The histograms show number of cells and HcRed fluorescence in the absence or presence of peptide. Traces correspond to mock-infected cells (Line C), mock-infected cells in the presence of peptides (Line D), SV40-infected cells (Line A), and SV40-infected cells in the presence of peptides (Line B).

FIG. 24 shows that biotinylated peptides inhibit HPV infection. HeLa cells were infected with HPV16 PsV at MOI of 1 in the presence or absence of 14 μM bP16/16. Infectivity was assessed 48 h.p.i. by flow cytometry for HcRed fluorescence.

FIG. 25 shows that L2 peptide does not inhibit internalization of HPV PsV. HeLa-S3 cells were incubated with 14 μM P16/16 or vehicle for one hour, followed by infection with HPV16 PsV at MOI of 50 at 4° C. for 2 h. Cells were washed with PBS to remove loosely-bound PsV and then shifted to 37° C. to allow virus internalization. After 8 or 16 h at 37° C., samples were fixed with 4% paraformaldehyde, permeabilized with 1% saponin, and stained with anti-L1 antibody. Cells were also stained with DAPI to visualize nuclei and examined by a Leica SP5 confocal microscope.

FIGS. 26A-26C show that L2 peptide does not inhibit protrusion of L2 protein into the cytoplasm. FIG. 26A depicts clonal HaCaT/GFP1-10NES cells expressing cytosolic GFP1-10 infected at MOI of 2000 with untagged HPV16 or with HPV16 PsV containing GFP11-tagged L2 in the absence or presence of 30 μM P16/16. Three h.p.i., cells were stained with Hoescht 33342 to visualize nuclei (blue) and examined by confocal microscopy for GFP fluorescence. FIG. 26B the corrected total cellular fluorescence (CTCF) of each cell was calculated and plotted in arbitrary units from three independent experiments. n.s., not significant. FIG. 26C depicts HaCaT/GFP-10NES cells infected as described in FIG. 26A with wild-type HPV16 PsV in the presence of 30 μM P16/16 or in its absence for three hours, followed by removal of PsV and incubation with medium containing peptide for an additional 48 hours. Cells were assessed by flow cytometry to determine reporter protein expression. Infectivity was normalized with that in the absence of peptide. **, p<0.01.

FIGS. 27A and 27B show that L2 peptide causes redistribution of DMT1-II from trans-Golgi network. FIG. 27A depicts HeLa-S3 cells transfected with a plasmid expressing a GFP-DMT1-II fusion protein. Six hours later, cells were treated with 14 μM P16/16 or PDM/16 or vehicle. After ˜20 hours, cells were fixed, permeabilized, and stained with anti-TGN46 antibody. Cells were stained with DAPI (blue) and imaged on a Leica SP5 inverted fluorescence microscope for GFP and anti-TGN46 fluorescence. Areas of colocalization are shown in yellow in the merge panels. FIG. 27B depicts Mander's coefficient for colocalization of staining was analyzed and plotted. ˜100 cells were analyzed for each condition.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, selected materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

As used herein, the term “cell-penetrating peptide” or “CPP” refers to a cell-permeable peptide, which is defined as a peptide capable of permeating and/or crossing a cell membrane. CPPs are sometimes referred to as protein transduction domains (PTD)

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

“L2” as used herein, refers to the minor capsid protein of a papillomavirus, of which the HPV16 type has the amino acid sequence (SEQ ID NO. 518):

```
              10 20 30 40 50
MRHKRSAKRT KRASATQLYK TCKQAGTCPP DIIPKVEGKT IAEQILQYGS
                60 70 80 90 100
MGVFFGGLGI GTGSGTGGRT GYIPLGTRPP TATDTLAPVR PPLTVDPVGP
              110 120 130 140 150
SDPSIVSLVE ETSFIDAGAP TSVPSIPPDV SGFSITTSTD TTPAILDINN
              160 170 180 190 200
TVTTVTTHNN PTFTDPSVLQ PPTPAETGGH FTLSSSTIST HNYEEIPMDT
              210 220 230 240 250
FIVSTNPNTV TSSTPIPGSR PVARLGLYSR TTQQVKVVDP AFVTTPTKLI
              260 270 280 290 300
TYDNPAYEGI DVDNTLYFSS NDNSINIAPD PDFLDIVALH RPALTSRRTG
              310 320 330 340 350
IRYSRIGNKQ TLRTRSGKSI GAKVHYYYDL STIDPAEEIE LQTITPSTYT
              360 370 380 390 400
TTSHAASPTS INNGLYDIYA DDFITDTSTT PVPSVPSTSL SGYIPANTTI
              410 420 430 440 450
PFGGAYNIPL VSGPDIPINI TDQAPSLIPI VPGSPQYTII ADAGDFYLHP
              460 470
SYYMLRKRRK RLPYFFSDVS LAA
```

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds can also be incorporated into the compositions. The “pharmaceutically acceptable carrier” can further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “polypeptide” refers to a polymer composed of amino acid residues, related naturally occurring structural variants, and synthetic non-naturally occurring analogs thereof linked via peptide (or amide) bonds. Synthetic polypeptides can be synthesized, for example, using an automated polypeptide synthesizer.

As used herein, the term “solvate” refers to a complex between a molecule and a solvent molecule, which can exist in solution or in solid phase. In certain embodiments, the solvent comprises at least one selected from the group consisting of water, methanol, ethanol, n-propanol, 2-propanol, DMSO, DMF, ethyl ether, acetone and pyridine.

As used herein, the term “transport construct” refers to a construct that crosses the cell membrane, wherein the construct comprises a cell-penetrating peptide and at least one heterologous cargo molecule, wherein the cargo molecule alone crosses the cell membrane at a lower rate or to a lower degree than the transport construct. In certain embodiments, the cargo molecule is selected from the group consisting of a nucleic acid; peptide; protein; peptide nucleic acid; oligosaccharide; lipid; glycolipid; lipoprotein; small molecule compound; therapeutic drug; UV-vis, fluorescent or radioactive label; imaging agent; diagnostic agent; prophylactic agent; liposome and virus. In other embodiments, the cargo molecule is linked to the transport peptide through a covalent or non-covalent linkage.

As used herein, “treating a disease or disorder” means reducing the frequency or severity with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that can be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Transport Constructs

The invention is based in part on the discovery that the human papillomavirus L2 protein C-terminal basic sequence acts as a cell-penetrating peptide that facilitates entry into the cell and escape from the endosome. These sequences are unlikely to be toxic because they naturally evolved to support infection by a non-lytic virus. There are a variety of analogous peptides found in various types of papillomavirus that can be fused to a cargo molecule and used for cell penetration and endosomal escape.

In one aspect, the invention provides an isolated transport construct comprising a cargo molecule and at least one cell-penetrating peptide selected from the group consisting of SEQ ID NO:1 to SEQ ID NO: 164, or a salt or solvate thereof. In various embodiments, the cell-penetrating peptide is SEQ ID NO: 2, which is RKRRKR. This is the basic sequence common to several papillomavirus L2 proteins including HPV16 and is evaluated in the examples described elsewhere herein. The sequences for other cell-penetrating peptides are shown in Table 1.

In various embodiments, the cargo molecule is selected from the group consisting of a nucleic acid; peptide; protein; peptide nucleic acid; oligosaccharide; lipid; glycolipid; lipoprotein; small molecule compound; therapeutic drug; UV-vis, fluorescent or radioactive label; imaging agent; diagnostic agent; prophylactic agent; liposome; and virus.

The cargo molecule can be combined with or linked to the cell-penetrating peptide to form the transport construct of the present invention. The cell-penetrating peptide and the cargo molecule are combined or linked in such a manner that they remain combined or linked under the conditions in which the transport construct is used (e.g., under conditions in which the transport construct is administered to an individual).

In certain embodiments, the cargo molecule is covalently linked to the cell-penetrating peptide through a linker or a chemical bond. In other embodiments, the linker comprises a disulfide bond, or the chemical bond between the cargo molecule and the transport peptide comprises a disulfide bond. In yet other embodiments, the cargo molecule comprises a peptide. In yet other embodiments, the transport peptide is covalently linked through an amide bond to the N-terminus of the peptide molecule of the cargo molecule. In yet other embodiments, the transport peptide is covalently linked through an amide bond to the C-terminus of the peptide of the cargo molecule. In yet other embodiments, the transport peptide is covalently linked through an amide bond to both the N-terminus and the C-terminus of the peptide of the cargo molecule.

Alternatively, the transport peptide and the cargo molecule are combined through a noncovalent linkage, such as electrostatic and/or hydrophobic interaction.

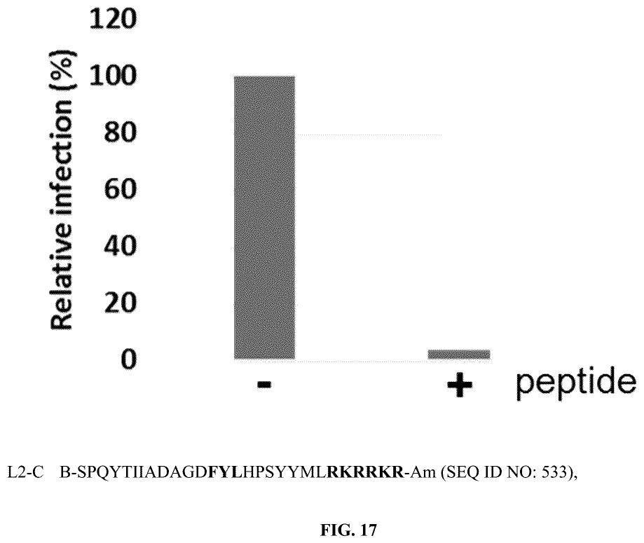

In various embodiments, the transport construct further comprises a linker connecting the cargo molecule and the cell-penetrating peptide. In various embodiments, the linker is a disulfide, a polyethylene glycol chain (PEG), a short polypeptide chain, or an amide, or other linkers that do not impair activity. In various embodiments, the cargo molecule can inhibit infection by papillomavirus. As shown in FIG. 17, HPV16 L2 CPP can deliver a peptide into cells that inhibits infection by HPV16 and other HPV types.

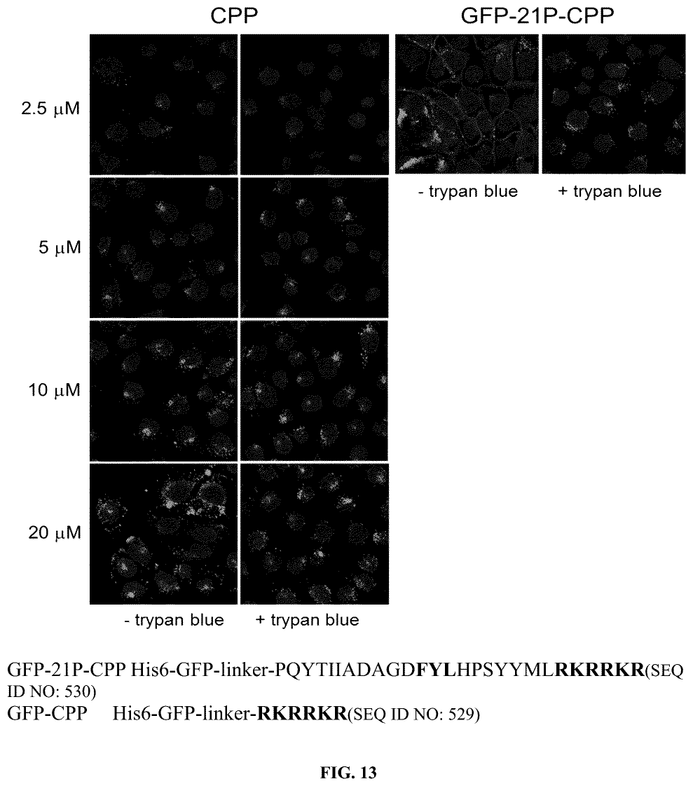

In various embodiments, the transport construct can include one or more flanking sequences that modulate the cell-penetrating activity or properties of the transport construct. In various embodiments, the flanking sequences can be an activity modulating flanking sequence, a flanking sequence that modulates temperature dependence, and/or a flanking sequence that modulates pH dependence, or a flanking sequence that modulates other aspects of cell penetration, such as lipid composition of the membrane. The term “flanking sequence” as used herein refers to a peptide fused to the transport construct that modulates the cell-penetrating activity of the transport construct. In various embodiments, the flanking sequence can modulate the cell-penetrating activity of the transport construct generally. As shown in FIG. 13, the activity modulating flanking sequence can be the 21 flanking upstream amino acids from L2, which improve the cell-penetrating activity of the construct relative to a transport construct with the cell-penetrating peptide alone.

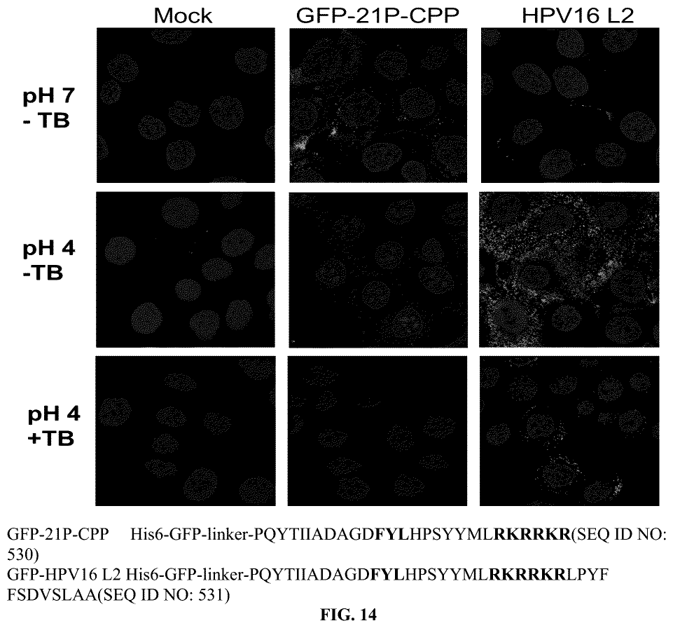

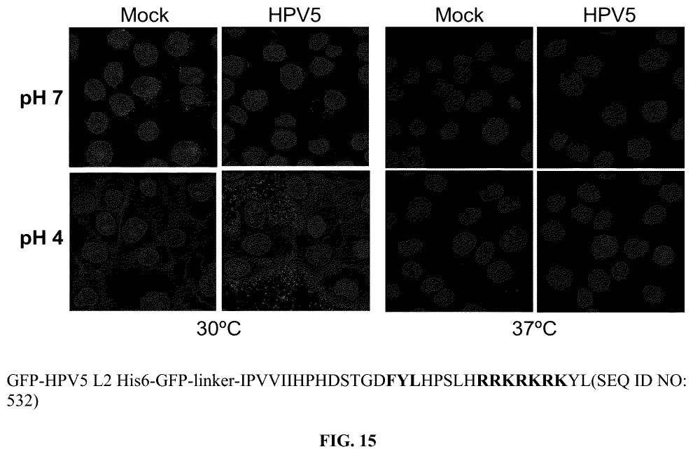

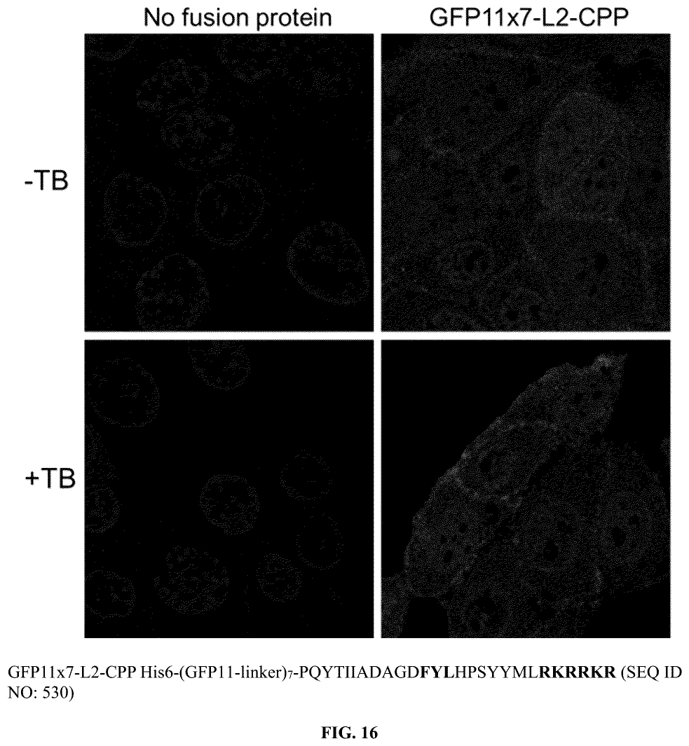

In various embodiments, the flanking sequence can be a certain portion of L2 from a form of papillomavirus selected to modulate the activity of the transport construct based on the characteristics of the papillomavirus. As shown in FIG. 14, addition of the 12 amino acids at the C-terminus of HPV16 L2 improves the cell-penetrating activity of the transport construct at pH 4. As a non-limiting example, HPV5 infects skin and therefore would require greater cell-penetrating activity at lower temperatures. As shown in FIG. 15, addition of the HPV5 C-terminus to the transport construct modulates the cell-penetrating activity such that it is higher at 30° C. than at 37° C.

In various embodiments, the invention provides a pharmaceutical composition comprising the transport construct as described herein and at least one pharmaceutically acceptable excipient.

### Methods of Delivering a Cargo Molecule

In another aspect, the invention provides a method of delivering a cargo molecule to the cytoplasm of at least one cell by contacting the cell with a transport construct comprising the cargo molecule and at least one cell-penetrating peptide selected from the group consisting of SEQ ID NO:1 to SEQ ID NO: 164, or a salt or solvate thereof.

In another aspect, the invention provides a method of promoting endosomal escape for a cargo molecule by contacting at least one cell with a transport construct comprising the cargo molecule and at least one cell-penetrating peptide selected from the group consisting of SEQ ID NO:1 to SEQ ID NO: 164. In various embodiments, the cell-penetrating peptide is SEQ ID NO: 2, or a salt or solvate thereof.

The cargo molecule can be any molecule intended for delivery to the cytoplasm and that can be covalently linked the cell-penetrating peptides of the invention. In various embodiments, the cargo molecule is at least one selected from the group consisting of a nucleic acid; peptide; protein; oligosaccharide; lipid; glycolipid; lipoprotein; small molecule compound; therapeutic drug; UV-vis, fluorescent or radioactive label; imaging agent; diagnostic agent; prophylactic agent; liposome; and virus.

The methods of the invention can be applied to deliver a cargo molecule to cells in vitro or to the cells of a subject. In various embodiments, the contacting step comprises administering a pharmaceutical composition comprising the transport construct and at least one pharmaceutically acceptable carrier to a subject. In various embodiments, the cell or cells are human. In various embodiments, the subject is human. Various suitable pharmaceutically acceptable carriers are described under administration/dosage/formulations.

In table 1, the left two columns list virus type and associated cell penetrating peptide. The right two columns compile the basic sequences found in the various virus-associated cell-penetrating peptides and their frequency.

TABLE 1

| Virus | Sequence | Basic sequence | Frequency |
|---|---|---|---|
| RaIPV1 | ... DGDISLTDLNYRQYFLHPSLMKGKRRKKRVSG SEQ. ID NO: 165. | SEQ. ID NO: 1. KRRKR | 17 |
| FcaPV4 | ... PISPPLPFDSVTATFDLHPGLKHRKRKRKHHGLT SEQ. ID NO: 166. | SEQ. ID NO: 2. RKRRKR | 16 |
| HPV144 | ... LDPAILIDVASDTYYIHPSLLKKGKKRKYSDIF SEQ. ID NO: 167. | SEQ. ID NO: 3. KRKRKR | 14 |
| BPV4 | ... PDIILNFEDDTATFFLHPSLLKKHKHNKHWFL SEQ. ID NO: 168. | SEQ. ID NO: 4. RRKRKR | 14 |
| BPV9 | ... PAIIIDFDEDTATFFLHPSLLKKHKHKHWFF SEQ. ID NO: 169. | SEQ. ID NO: 5. RRRRKR | 13 |
| HPV62 | ... PYSIYIVGSDYYLFPSYIFFPKKHKRLHYFFTDGYVAAW | SEQ. ID NO: 6. KKRKR | 12 |
| | SEQ. ID NO: 170. | | |
| HPV81 | ... PYAIYVVGTDFYLFPSYIFFPKKHKRIHYSFTDGYVAAW | SEQ. ID NO: 7. RRRKRKR | 12 |
| | SEQ. ID NO: 171. | | |
| CePV1 | ... ILTDSDAGFFWNTFLHPSLLSKKKGKKTF SEQ. ID NO: 172. | SEQ. ID NO: 8. RKRKR | 10 |
| HPV156 | ... QPSFGVDVYSDDFYLHPGLYHKKKKETNRIFLMFCRCPYGCRPLENC | SEQ. ID NO: 9. KRRKRKR | 6 |
| | IFHLKSQLLKC SEQ. ID NO: 173. | | |
| CcrPV1 | ... TPAVVIDLLGGTDFYLHPALFKKKKKRLFCDFFADGGVASCTE | SEQ. ID NO: 10. RKKRKR | 6 |
| | SEQ. ID NO: 174. | | |
| EcPV8 | ... PSTANSLDASYSYYLHPSLNNKKKKKSKGLRGGWWFVADDLLAT | SEQ. ID NO: 11. RKRKRKR | 6 |
| | SEQ. ID NO: 175. | | |
| HPV37 | ... TPTVVIRFGEAGTDYYLHPSLKKKKRKRKYL SEQ. ID NO: 176. | SEQ. ID NO: 12. RRRRRKR | 6 |
| CdPV1 | ... GIVIDLSDDYYRHYYLHPSLLKKKKSKVRKLWYA SEQ. ID NO: 177. | SEQ. ID NO: 13. RKRRKRK | 5 |
| OaPV3 | ... IPVSPAVSLGGANYWLEPSLIKKKRKKKRLI SEQ. ID NO: 178. | SEQ. ID NO: 14. RKRRR | 5 |
| HPV162 | ... QPPTVILDLFSDDYFLHPSYLKKKRKRSDIF SEQ. ID NO: 179. | SEQ. ID NO: 15. RRRRKRK | 5 |
| TmPV1 | ... TILIDFQSSYGDFFLHPSLIPKKKRRLGLFTDEYVVTE | SEQ. ID NO: 16. KKRKRK | 4 |
| | SEQ. ID NO: 180. | | |
| AmPV4 | ... TPVIVLDWQQSNDFFLHPSLIKKKRRKRSAAFF SEQ. ID NO: 181. | SEQ. ID NO: 17. RKRKKRK | 4 |
| HPV132 | ... IGPSYYVGVDNDFYLHPSLIPKKKRRRLDYF SEQ. ID NO: 182. | SEQ. ID NO: 18. RKRKRR | 4 |
| BPV22 | ... PIRPGLDVYDSIDFYLHPSLGKKLRKKRKRRFY SEQ. ID NO: 183. | SEQ. ID NO: 19. RKRRK | 4 |
| HPV68 | ... TFAITIYGSNYYLLPLLFFLLKKRKHLPYFFTDGIVAS | SEQ. ID NO: 20. RRRKRKRK | 4 |
| | SEQ. ID NO: 184. | | |
| RrPV1 | ... PWVVDGDGGSGYWIDPSLLTNKKRKKHFH SEQ. ID NO: 185. | SEQ. ID NO: 21. RRRRKRKR | 4 |
| CmPV1 | ... VHPAYSVTFSMLSELDDPFLTKKRKKCFADGCLDTFY | SEQ. ID NO: 22. RRRRKRR | 4 |
| | SEQ. ID NO: 186. | | |
| HPV18 | ... TQYIGIHGTHYYLWPLYYFIPKKRKRVPYFFADGFVAA | SEQ. ID NO: 23. KRRKRK | 3 |
| | SEQ. ID NO: 187. | | |
| HPV39 | ... TYAITIQGSNYYLLPLLYFFLKKRKRIPYFFSDGYVAV | SEQ. ID NO: 24. KRRRKR | 3 |
| | SEQ. ID NO: 188. | | |
| HPV45 | ... TTYIGIHGTQYYLWPWYYYFPKKRKRIPYFFADGFVAA | SEQ. ID NO: 25. RKKRKRK | 3 |
| | SEQ. ID NO: 189. | | |
| HPV70 | ... TVAIAIQGSNYYLLPLLYYFLKKRKRIPYFFTDGFVAV | SEQ. ID NO: 26. RKRKRK | 3 |
| | SEQ. ID NO: 190. | | |
| HPV71 | ... PSSFIVYGTEYYLMPSYIFFPKKRKRVHYFFADGFVAA | SEQ. ID NO: 27. RKRRRKR | 3 |
| | SEQ. ID NO: 191. | | |
| HPV83 | ... IQSVFIDGTDYYLLPNYIFFPKKRKRVHYSFADGYVAAW | SEQ. ID NO: 28. RRKKRKR | 3 |
| | SEQ. ID NO: 192. | | |
| HPV84 | ... PYAIYILGSDYYLLPNYIFFPKKRKRVPYSFSDGFVAAW | SEQ. ID NO: 29. RRRRK | 3 |
| | SEQ. ID NO: 193. | | |
| HPV85 | ... THSVVLQGTNYYLWPNYYFIFKKRKRVPYFLTDGFVAF | SEQ. ID NO: 30. RRRRRRRRRR | 3 |
| | SEQ. ID NO: 194. | | |
| HPV90 | ... PGSVLVNGSTYYLLPPLGLLPKKRKRFPYFFADGNVEA | SEQ. ID NO: 31. KKHKR | 2 |
| | SEQ. ID NO: 195. | | |
| HPV97 | ... THYIGINGTHYYLWPLYYFLPKKRKRVPYFFADGLLAA | SEQ. ID NO: 32. KKRKK | 2 |
| | SEQ. ID NO: 196. | | |
| HPV102 | ... SQSVFVDGTDYFLLPNYLFFPKKRKRVHYSFADGFVATW | SEQ. ID NO: 33. KKRKRKR | 2 |
| | SEQ. ID NO: 197. | | |
| EePV1 | ... TIVIEAFDSSGGFYLHPSFVGKKRKRLYFL SEQ. ID NO: 198. | SEQ. ID NO: 34. KRHKHK | 2 |
| EhPV1 | ... VPIIVVDATSQDFYLHPYLQHKKRKRKHFVYMFADGSVASEYQ | SEQ. ID NO: 35. KRKKRR | 2 |
| | SEQ. ID NO: 199. | | |
| HPV147 | ... VAPAVHVEAFGSNYYLHPSLLKKRKRKYLDVF SEQ. ID NO: 200. | SEQ. ID NO: 36. KRKRRKRK | 2 |
| CPV1 | ... PAIVIDILDSSADYYLHPSLIKKRKRKHFFF SEQ. ID NO: 201. | SEQ. ID NO: 37. KRKRRR | 2 |
| LrPV1 | ... PAILIEIWDSGSNYSLHPSLLKKRKRKLLFL SEQ. ID NO: 202. | SEQ. ID NO: 38. KRLKRKRK | 2 |
| HPV115 | ... LPAVVIHIYDDSGDFYLHPSLKKRKRKRAYL SEQ. ID NO: 203. | SEQ. ID NO: 39. KRRKRKRK | 2 |
| HPV170 | ... GPSILIDEFSSEDFVLHPSLSKKRKRKRLYSDF SEQ. ID NO: 204. | SEQ. ID NO: 40. KRRRRKR | 2 |
| HPV17 | ... TPTVVIKFAEAGGRFLFTPSLKKRKRKRKYL SEQ. ID NO: 205. | SEQ. ID NO: 41. RKHKHK | 2 |
| HPV158 | ... PFIPSTLQFDTFDYDIHPSAIKKRRKRKRSDFM SEQ. ID NO: 206. | SEQ. ID NO: 42. RKKRRR | 2 |
| HPV148 | ... VAPSYIPDYYADFYLYPSFLPKKRRRIDIV SEQ. ID NO: 207. | SEQ. ID NO: 43. RKRKRKRK | 2 |
| EcPV4 | ... SATPPTSTDTAGFSMLDPSLLKRCRRKRRSCFADGIVDARQSEVLSAP | SEQ. ID NO: 44. RKRRKRKR | 2 |
| | CSCDKGAQY SEQ. ID NO: 208. | | |
| BPV3 | ... PDIIITFEEGTATFFLHPSLLKRHKHKHWFF SEQ. ID NO: 209. | SEQ. ID NO: 45. RKRRRKRK | 2 |
| BPV15 | ... PDIVIDMEEDTATFTLHPSLLKRHKHKHWFF SEQ. ID NO: 210. | SEQ. ID NO: 46. RRKRKRK | 2 |
| HPV159 | ... PTVVIHIHDTSGDYYLHPSLQKRKCKRKHRYL SEQ. ID NO: 211. | SEQ. ID NO: 47. RRKRKRKR | 2 |
| HPV135 | ... GPTSPVSFYSDDFYLHPSLFTKRKKRKYYNF SEQ. ID NO: 212. | SEQ. ID NO: 48. RRKRR | 2 |
| BPV5 | ... ITDSGVDGTYFLNTYLHPSLHKRKKRRFS SEQ. ID NO: 213. | SEQ. ID NO: 49. RRRRRKRK | 2 |
| BPV8 | ... IIDFSVEGTYFLNTYAHPSLHKRKKRRLS SEQ. ID NO: 214. | SEQ. ID NO: 50. RRRRRRRR | 2 |
| AmPV2 | ... TPIITGVFVSFDFWLHPSQLLKRKRSPFYLADGIVAA | SEQ. ID NO: 51. KGKRRKKR | 1 |
| | SEQ. ID NO: 215. | | |
| HPV128 | ... PYAPSPITVFGDTFYLNPSLLKRKRKQYFY SEQ. ID NO: 216. | SEQ. ID NO: 52. KHRKRKRK | 1 |
| HPV73 | ... PAGPIYIYGSGFILHPSYYLLKRKRKRLSYSFTDVATY | SEQ. ID NO: 53. KKGKKRK | 1 |
| | SEQ. ID NO: 217. | | |
| HPV104 | ... PAVVIHIADASGDFYLHPSLQKRKRKRAYL SEQ. ID NO: 218. | SEQ. ID NO: 54. KKHK | 1 |
| HPV121 | ... EPAIIIDLESASDFFIHPSLLKRKRKRPLL SEQ. ID NO: 219. | SEQ. ID NO: 55. KKHKHK | 1 |
| HPV130 | ... EPAIIIDLESANDFLIHPSLLKRKRKRPLL SEQ. ID NO: 220. | SEQ. ID NO: 56. KKKGKK | 1 |
| HPV133 | ... EPAIVIDLESASDFFIHPSLLKRKRKRPLL SEQ. ID NO: 221. | SEQ. ID NO: 57. KKKK | 1 |
| HPV136 | ... INPSLGASVIFDTYDLHPSLLKRKRKRPFF SEQ. ID NO: 222. | SEQ. ID NO: 58. KKKKKR | 1 |
| HPV142 | ... EPAIIIDVDSATDFLIHPSLLKRKRKRHLL SEQ. ID NO: 223. | SEQ. ID NO: 59. KKKKKSK | 1 |
| HPV153 | ... PLYPTEVTVYGDSFVIDPFFLKRKRKRYTLY SEQ. ID NO: 224. | SEQ. ID NO: 60. KKKKRKRK | 1 |
| HPV166 | ... TEPHLIIDLFSDDFYLHPGYLKRKRKRSDIF SEQ. ID NO: 225. | SEQ. ID NO: 61. KKKKSKVR | 1 |
| HPV175 | ... IMPGVTIDIYSIDYDIHPSLLKRKRKRIDYV SEQ. ID NO: 226. | SEQ. ID NO: 62. KKKKKKR | 1 |
| HPV180 | ... EPAIIIDLESATDFLIHPSLLKRKRKRNLL SEQ. ID NO: 227. | SEQ. ID NO: 63. KKKRKR | 1 |
| UuPV1 | ... AIVIEIWGSGNSYSLHPSLLSKRKRKRLSL SEQ. ID NO: 228. | SEQ. ID NO: 64. KKKRR | 1 |
| TmPV3 | ... HIFVYSPHLTSFDFLPHPSLLKRKRKRSLDDDFTILQ | SEQ. ID NO: 65. KKKRRKR | 1 |
| | SEQ. ID NO: 229. | | |
| GcPV1 | ... IIDNELAVWFHSYFLHPSKLGKRKRKRSDSSV SEQ. ID NO: 230. | SEQ. ID NO: 66. KKKRRR | 1 |
| BPV14 | ... IIDGNTIDLYSSNFTLHPSLLKRKRKRKHA SEQ. ID NO: 231. | SEQ. ID NO: 67. KKLRKKRKRR | 1 |
| HPV111 | ... PTVVIHISDTSGDYYLHPSLQKRKRKRKRKYL SEQ. ID NO: 232. | SEQ. ID NO: 68. KKRK | 1 |
| MpPV1 | ... PTIIVTKETGVTYDLHPSLFGKRKRKRRSL SEQ. ID NO: 233. | SEQ. ID NO: 69. KKRKRKRK | 1 |
| RtiPV1 | ... TVLINVYDEGLDFLLHPSMFPKRKRRKLAFL SEQ. ID NO: 234. | SEQ. ID NO: 70. KKRRKRKR | 1 |
| EIPV1 | ... PGILIEVLDSSGDFYLHPSLLKRKRRKRPSF SEQ. ID NO: 235. | SEQ. ID NO: 71. KKRRR | 1 |
| HPV122 | ... PTVVIHIADTSGDYYLHPSLQKRKRRKRKYL SEQ. ID NO: 236. | SEQ. ID NO: 72. KRCRRKRR | 1 |
| CgPV2 | ... PVVIIYPHDNTGDFYLHPSLHKRKRRKRKYF SEQ. ID NO: 237. | SEQ. ID NO: 73. KRKCKRK | 1 |
| HPV165 | ... PAPHTVSDINDDYYLYPSLYPKRKRRRLDFF SEQ. ID NO: 238. | SEQ. ID NO: 74. KRKKRK | 1 |
| HPV184 | ... PLEPANAIEVYYDFFLHPALQKRKRRRLDVF SEQ. ID NO: 239. | SEQ. ID NO: 75. KRKR | 1 |
| CcaPV1 | ... DGNINLQEVLYRQYFLHPSLMKRKRRRKRLFG SEQ. ID NO: 240. | SEQ. ID NO: 76. KRKRK | 1 |
| RnPV2 | ... RPPIYIGSSPGVDYYLHPSLYKRKRRRRRHSYL SEQ. ID NO: 241. | SEQ. ID NO: 77. KRKRKRK | 1 |
| HPV20 | ... PVVIIHTYDTSGDFYLHPSLTKRLKRKRKYL SEQ. ID NO: 242. | SEQ. ID NO: 78. KRKRKRKRK | 1 |
| HPV14 | ... PVVIIHTYDTSGDFYLHPSLHKRLKRKRKYL SEQ. ID NO: 243. | SEQ. ID NO: 79. KRKRKRR | 1 |
| CcPV1 | ... STPLYPPRHVFFSDLDDPIMFKRRKKCFADGCVDAFY | SEQ. ID NO: 80. KRKRRK | 1 |
| | SEQ. ID NO: 244. | | |
| AsPV1 | ... PPTVIVYDYDDSVDFYLHPSLKRRKKRKYIVY SEQ. ID NO: 245. | SEQ. ID NO: 81. KRKRRKR | 1 |
| HPV2 | .. PSSVYIFGGDYYLMPSYVLWPKRRKRVHYFFADGFVAA | SEQ. ID NO: 82. KRKRRRKR | 1 |
| | SEQ. ID NO: 246. | | |
| HPV26 | ... LPAIVVHGDNYYLWPYIYLIHKRRKRMPYFFSDGFVAY | SEQ. ID NO: 83. KRKRRRRR | 1 |
| | SEQ. ID NO: 247. | | |
| HPV27 | ... PSSVYIFGGDYYLLPSYILWPKRRKRVNYFFADGFVAA | SEQ. ID NO: 84. KRRKK | 1 |
| | SEQ. ID NO: 248. | | |
| HPV57 | ... PSSVYIVGGDYYLLPSYVLWPKRRKRVHYFFADGYVAA | SEQ. ID NO: 85. KRRKKRK | 1 |
| | SEQ. ID NO: 249. | | |
| HPV61 | ... PHSIYIQGSDFYLLPAYVFFPKRRKRVPYSFSDGFVAAW | SEQ. ID NO: 86. KRRR | 1 |
| | SEQ. ID NO: 250. | | |
| HPV72 | ... PHSIYVEGFDFYLLPAYIFFPKRRKRVPYSFADGFVAAW | SEQ. ID NO: 87. KRRRK | 1 |
| | SEQ. ID NO: 251. | | |
| HPV87 | ... PYSVYIQGSDYYLLPNYIFFPKRRKRVPYSFSDGFVAAW | SEQ. ID NO: 88. KRRRR | 1 |
| | SEQ. ID NO: 252. | | |
| HPV91 | ... PSAVSIYGTDFYLHPSLLHFGKRRKRISYFFADNYVAA | SEQ. ID NO: 89. KRRRRR | 1 |
| | SEQ. ID NO: 253. | | |
| HPV114 | ... PYAIYIVGSDYYLLPNYIFFPKRRKRVPYSFSDGFVAAW | SEQ. ID NO: 90. KRRRRRR | 1 |
| | SEQ. ID NO: 254. | | |
| CgPV1 | ... QHSIYVHGTDFYLLPGYLFVPKRRKRFIYSFADGYVAA | SEQ. ID NO: 91. KRRRRRRRK | 1 |
| | SEQ. ID NO: 255. | | |
| AgPV1 | ... QPSAAISIFASDFYLHPSYILKRRKRVPYTFFADGIVAS | SEQ. ID NO: 92. KRRRRRRTRR | 1 |
| | SEQ. ID NO: 256. | | |
| SscPV1 | ... APPASVTVVSGDFVLHPSYFWKRRKRVSYFFADGVVAA | SEQ. ID NO: 93. KSRKRK | 1 |
| | SEQ. ID NO: 257. | | |
| SscPV2 | ... VIPAAVTIYAGDFFLHPSYIWKRRKRVSYFLADGIVAA | SEQ. ID NO: 94. KTRKRKRK | 1 |
| | SEQ. ID NO: 258. | | |
| TePV1 | ... PVIVIHGVYFSVDFYLHPHLLKRRKRFHF SEQ. ID NO: 259. | SEQ. ID NO: 95. KWKKRKR | 1 |
| HPV43 | ... PQSVSIHGTDFYLHPSLWHLGKRRKRFSYFFTDNYVAA | SEQ. ID NO: 96. RGRKRKRR | 1 |
| | SEQ. ID NO: 260. | | |
| MfPV6 | ... IGHVVVHGGDFYLHPSYYTLHKRRKRMPRFLADVSVAA | SEQ. ID NO: 97. RHRRKRR | 1 |
| | SEQ. ID NO: 261. | | |
| HPV89 | ... PQAIYIQGTDFYLVPNYVFFPKRRKRVPYSFADGFVAAW | SEQ. ID NO: 98. RHRRRKR | 1 |
| | SEQ. ID NO: 262. | | |
| HPV112 | ... YTPVIYIDPFGSDFYLHPALLKRRKRKYSEIF SEQ. ID NO: 263. | SEQ. ID NO: 99. RKFKRRRK | 1 |
| HPV172 | ... LIPGRSVDVYSNDFIIHPSVLKRRKRKLSDSF SEQ. ID NO: 264. | SEQ. ID NO: 100. RKHRHK | 1 |
| HPV168 | ... ISPAVLIDAFSADYYLHPSLMKRRKRKYSEIF SEQ. ID NO: 265. | SEQ. ID NO: 101. RKKKKK | 1 |
| HPV150 | ... TPTVIIHTEDYSGDYYLHPSLKRRKRKRAYL SEQ. ID NO: 266. | SEQ. ID NO: 102. RKKKKRK | 1 |
| HPV169 | ... PVGPSSSVDIFYYDYDLHPSLKRRKRKRNMF SEQ. ID NO: 267. | SEQ. ID NO: 103. RKKKR | 1 |
| HPV171 | ... PIGPSNSVEIFYYDYDLHPSLKRRKRKRNVF SEQ. ID NO: 268. | SEQ. ID NO: 104. RKKRK | 1 |
| PcPV1 | ... PAIILEIWGSGENYSLHPSLLKRRKRKRLIL SEQ. ID NO: 269. | SEQ. ID NO: 105. RKKRR | 1 |
| CPV17 | ... DVPSSSSDSVSSTFDLHPSLLKRRKRKRGDI SEQ. ID NO: 270. | SEQ. ID NO: 106. RKKRRK | 1 |
| HPV197 | ... LEPPILLDYLGSGYYLHPSLWKRRKRKRSDTYNSFTDGIVDATEW | SEQ. ID NO: 107. RKKRRKR | 1 |
| | SEQ. ID NO: 271. | | |
| HPV80 | ... TPTWVINFEDAGGDYYLHPSLKRRKRKRKYL SEQ. ID NO: 272. | SEQ. ID NO: 108. RKKRRKRK | 1 |
| HPV164 | ... ITPAVYLDAFSSDYYLHPALMKRRKRKRKYLEVF SEQ. ID NO: 273. | SEQ. ID NO: 109. RKKRRRR | 1 |
| HPV50 | ... SEQPLFVLDYSDYDLHPGLLPKRRRIDYF SEQ. ID NO: 274. | SEQ. ID NO: 110. RKRFKRKRK | 1 |
| MrPV1 | ... IFSTPYYEYNYILDPSILFLLKRRRKLFV SEQ. ID NO: 275. | SEQ. ID NO: 111. RKRK | 1 |
| HPV31 | ... PQVSIFVDGGDFYLHPSYYMLKRRRKRVSYFFTDVSVAA | SEQ. ID NO: 112. RKRKHK | 1 |
| | SEQ. ID NO: 276. | | |
| HPV35 | ... PIYSIIADGGDFYLHPSYYLLKRRRKRIPYFFADVSVAV | SEQ. ID NO: 113. RKRKKRR | 1 |
| | SEQ. ID NO: 277. | | |
| HPV69 | ... SHSVVAQGGNYYLWPYIYLIHKRRRKRVPCFFSDGLAAY | SEQ. ID NO: 114. RKRKRKYR | 1 |
| | SEQ. ID NO: 278. | | |
| EaPV1 | ... PSFDWPMTDLDADFVLHPSLLKRRRRFYWSFADGGLASRTK | SEQ. ID NO: 115. RKRKRRRK | 1 |
| | SEQ. ID NO: 279. | | |
| HPV53 | ... DTTHDVVIQGSTFALWPVYFLKRRRRKRIPYFLADGGVAA | SEQ. ID NO: 116. RKRR | 1 |
| | SEQ. ID NO: 280. | | |
| HPV66 | ... DVTHDVYIQGATFALWPVYFFKRRRRKRIPYFFADGDVAA | SEQ. ID NO: 117. RKRRKK | 1 |
| | SEQ. ID NO: 281. | | |
| TmPV2 | ... PIVIIDLTSTSIDYFLHPSLAKRRRRRAHWSFLADVGLAT | SEQ. ID NO: 118. RKRRKRR | 1 |
| | SEQ. ID NO: 282. | | |
| RtPV2 | ... PDIIIDFLTPGDTFYLHPSHFKRRRRRRYQLFFF SEQ. ID NO: 283. | SEQ. ID NO: 119. RKRRRRRKR | 1 |
| CPV3 | ... VIVDVERGSGSDYYLHPSLSLKRRRRRRRKSL SEQ. ID NO: 284. | SEQ. ID NO: 120. RKRRRRRRRR | 1 |
| MscPV1 | ... ITAIPAGGQSIDFLLHPGLFPKRRRRRRTRRHSYL | SEQ. ID NO: 121. RLKRKRK | 1 |
| | SEQ. ID NO: 285. | | |
| HPV119 | ... ITPLIQIDPFGPDFYLHPALMKSRKRKYLEVF SEQ. ID NO: 286. | SEQ. ID NO: 122. RLKRKRKR | 1 |
| HPV15 | ... TPTVVINFEEAGGDYYLHPSLKTRKRKRKYL SEQ. ID NO: 287. | SEQ. ID NO: 123. RLKRRRRR | 1 |
| HPV92 | ... TPTVIIHTEDFSGDYYLHPSLKWKKRKRAYL SEQ. ID NO: 288. | SEQ. ID NO: 124. RLRKRRKRR | 1 |
| PmPV1 | ... PQPPTYEGPSSGVTYYLHPSLRGRKRKRRNLHVRFSIPDGILAS | SEQ. ID NO: 125. RLRRKR | 1 |
| | SEQ. ID NO: 289. | | |
| FcaPV5 | ... VMPPIIFDPETSTFDLHPSLHRHRRKRRHIGL SEQ. ID NO: 290. | SEQ. ID NO: 126. RLRRKRKR | 1 |
| HPV10 | ... THYVYIDGGDFYLWPVTFHFSRHRRRKRVSYFFADGTLAL | SEQ. ID NO: 127. RRKKRKRK | 1 |
| | SEQ. ID NO: 291. | | |
| HPV21 | ... PVVIIHTFDTSGDFYLHPSLSRKFKRRRKYL SEQ. ID NO: 292. | SEQ. ID NO: 128. RRKRK | 1 |
| BPV10 | ... PTVIIDFEDGSATFFLHPSLLRKHKHKHWFF SEQ. ID NO: 293. | SEQ. ID NO: 129. RRKRKKRKR | 1 |
| BPV11 | ... PEIVIDFEENTATFYLHPSLLRKHKHKHWFF SEQ. ID NO: 294. | SEQ. ID NO: 130. RRKRKKRKRR | 1 |
| BPV6 | ... PDIIVNLEENTATFFLHPSLLRKHRHKHWFF SEQ. ID NO: 295. | SEQ. ID NO: 131. RRKRKRR | 1 |
| MscPV2 | ... PLVIIDPVGTGANYFLHPSLLRKKKKKLIFH SEQ. ID NO: 296. | SEQ. ID NO: 132. RRKRRKRKR | 1 |
| MsPV1 | ... EVVIVQEGNNSGTFYLHPSLLRKKKKRKYVF SEQ. ID NO: 297. | SEQ. ID NO: 133. RRKRRKRR | 1 |
| AmPV1 | ... WYPTILSELSSSDFIFHPSLWRKKKRFPFFLSDGIVAA | SEQ. ID NO: 134. RRKRRRR | 1 |
| | SEQ. ID NO: 298. | | |
| RaPV1 | ... VIVEDDTAAYDYWFDLYLHLPRKKRKWCSFCSLTDGIVDT | SEQ. ID NO: 135. RRKRRRRGRR | 1 |
| | SEQ. ID NO: 299. | | |
| MfPV9 | ... PTTHVVVYGGDFYLHPSYFPVRKKRKRVHRFLSDVIVAA | SEQ. ID NO: 136. RRKRRRRKR | 1 |
| | SEQ. ID NO: 300. | | |
| HPV4 | ... LEPALLSDIFSTDFVYRPSLYRKKRKRLEMF SEQ. ID NO: 301. | SEQ. ID NO: 137. RRKRRRRRK | 1 |
| HPV65 | ... LEPPFFSEFYSSDFVYRPSLYRKKRKRSDIF SEQ. ID NO: 302. | SEQ. ID NO: 138. RRKRRRRRRKR | 1 |
| HPV95 | ... PLKPALLTDFYSDFTYYPSLYRKKRKRSDLF SEQ. ID NO: 303. | SEQ. ID NO: 139. RRLRRKRK | 1 |
| PphPV1 | ... PLIVLFEPGFGPSFYLHPSLLRKKRKRVFY SEQ. ID NO: 304. | SEQ. ID NO: 140. RRRGRKRKR | 1 |
| TtPV5 | ... PFIFFLFSHGDPSFFLHPSLLRKKRKRVFY SEQ. ID NO: 305. | SEQ. ID NO: 141. RRRKRIK | 1 |
| HPV134 | ... RPSILIDDFASNDFVLHPSLNRKKRKRKQVHFL SEQ. ID NO: 306. | SEQ. ID NO: 142. RRRKRK | 1 |
| HPV9 | ... PTVVIHINDTSGDYYLHPSLQRKKRKRKYL SEQ. ID NO: 307. | SEQ. ID NO: 143. RRRKRR | 1 |
| EhPV3 | ... VGPSYVESTDSFPYWLAPSLQRKKRKRKTVSFCSLADGCMDS | SEQ. ID NO: 144. RRRKRRKRR | 1 |
| | SEQ. ID NO: 308. | | |
| HPV48 | ... INWFPLFDSYSDFALDPFFIPRKKRRLDIL SEQ. ID NO: 309. | SEQ. ID NO: 145. RRRR | 1 |
| SfPV1 | ... ISPVFIFEGNADGTYYLEEPLRKKRRKSIFLLADGSVAVYAE | SEQ. ID NO: 146. RRRRARR | 1 |
| | SEQ. ID NO: 310. | | |
| CPV6 | .. PAISFDIYGDGLNFYLHPSLLRKKRRKRYFY SEQ. ID NO: 311. | SEQ. ID NO: 147. RRRRKRKRR | 1 |
| HPV23 | ... APAVVIHTLDKSFDYYLHPSLRKKRRKRKYL SEQ. ID NO: 312. | SEQ. ID NO: 148. RRRRKRRR | 1 |
| HPV127 | ... TPAFYLDIYNDFNLHPALLPPRKKRRRLDIF SEQ. ID NO: 313. | SEQ. ID NO: 149. RRRRRK | 1 |
| HPV199 | ... LTPSIITSSYNNFYLEPFYVPRKKRRRLDMF SEQ. ID NO: 314. | SEQ. ID NO: 150. RRRRRKRRR | 1 |
| TtPV2 | ... PNINVYSLDDTGGQFRFWHFLRKKRRRRYL SEQ. ID NO: 315. | SEQ. ID NO: 151. RRRRRR | 1 |
| HPV19 | ... LPVVIIHTYDTSGDFYLHPSLRKRFKRKRKYL SEQ. ID NO: 316. | SEQ. ID NO: 152. RRRRRRKR | 1 |
| BPV7 | ... VVVISYTADVSIFSLFEPSLYRKRKYSYLY SEQ. ID NO: 317. | SEQ. ID NO: 153. RRRRRRR | 1 |
| BPV23 | ... PEIVIDFGESTASFSLHPSLLRKRKHKHWFF SEQ. ID NO: 318. | SEQ. ID NO: 154. RRRRRRRRK | 1 |
| BPV1 | ... IIDGHTVDLYSSNYTLHPSLLRKRKKRKHA SEQ. ID NO: 319. | SEQ. ID NO: 155. RRRRRRRRRK | 1 |
| BPV2 | ... IIDGHTVDLYSNNYSLHPSLLRKRKKRKHA SEQ. ID NO: 320. | SEQ. ID NO: 156. RSKKRKRK | 1 |
| BPV13 | ... IIDGHTIDLYSNNYSLHPSLLRKRKKRKHA SEQ. ID NO: 321. | SEQ. ID NO: 157. RSRKRKR | 1 |
| BgPV1 | ... IIDGHIVDLYSRNYSLHPSLYRKRKKRKHA SEQ. ID NO: 322. | SEQ. ID NO: 158. RTKRRKRK | 1 |
| BPV16 | ... DYSYDQSAGPSFTLDPSLLQLRKRKKRRYY SEQ. ID NO: 323. | SEQ. ID NO: 159. RVKKRR | 1 |
| HPV34 | ... SGPIYIYGSDFILHPSLYVIPRKRKRLSYFFADVATY | SEQ. ID NO: 160. RWKKRKRK | 1 |
| | SEQ. ID NO: 324. | | |
| HPV106 | ... TGDVLVHGSTYYLLPSYVLLPRKRKRFPSFFADGIVEA | SEQ. ID NO: 161. RWRKRKR | 1 |
| | SEQ. ID NO: 325. | | |
| HPV59 | ... IQSINIEGTNYFLWPIYYFLPRKRKRVPYFFTDGSMAF | SEQ. ID NO: 162. RWRKRKRK | 1 |
| | SEQ. ID NO: 326. | | |
| HPV86 | ... PYAIYIQGSDYYLLPNYIFFPRKRKRVHYSFSDGFVAAW | SEQ. ID NO: 163. RWRRKRRRR | 1 |
| | SEQ. ID NO: 327. | | |
| AaPV1 | ... GNINVSMEYFRHYYLHPSLLGRKRKRLFG SEQ. ID NO: 328. | SEQ. ID NO: 164. RWRRRKRK | 1 |
| HPV103 | ... PSVVVDSFVTSTTFYLHPGLSRKRKRSHMF SEQ. ID NO: 329. | | |
| HPV129 | ... QPSFAIDVDNSTFDIHPSLLPRKRKRPSF SEQ. ID NO: 330. | | |
| HPV140 | ... LGPAVGLTVNFDDFDLHPSLLRKRKRSIL SEQ. ID NO: 331. | | |
| HPV202 | ... LGPSQGLTVDFYDFDLHPSLLRKRKRSIF SEQ. ID NO: 332. | | |
| HPV204 | ... PLITIRIDNSTGDYDVDPSLLRKRKRVF SEQ. ID NO: 333. | | |
| PphPV4 | ... ISPAFWNMSYSTFYLHPGYLARKRKRKVLT SEQ. ID NO: 334. | | |
| McPV2 | ... IPARTVIYFDDFADFYLHPSLRKRKRKHVIR SEQ. ID NO: 335. | | |
| MmiPV1 | ... PGADVVVVPEDSVDYYLHPSLRKRKRKYVFH SEQ. ID NO: 336. | | |
| CPV2 | ... IPLSSPDYGSVTFDLHPGLIHRKRKRKRGSV SEQ. ID NO: 337. | | |
| BpPV1 | ... PFEPPTEPDPAETGGNFEPSLRKRKRKRVEVASRTYSLRRKR | | |
| | SEQ. ID NO: 338. | | |
| HPV123 | ... TPSILVDGFSSTDFILHPSHTRKRKRKRSDYF SEQ. ID NO: 339. | | |
| HPV161 | ... DNTPLLVLDLTSDDYFLHPSLRKRKRKRSDVF SEQ. ID NO: 340. | | |
| FcaPV1 | ... PAIVIEVWASGATYSLHPSLLRKRKRKRISL SEQ. ID NO: 341. | | |
| PIpPV1 | ... PAIVIDIWESGNSYSLHPSLLRKRKRKRLFL SEQ. ID NO: 342. | | |
| HPV105 | ... IPIVVIHTHDSSGDFYLHPSLRKRKRKRKYL SEQ. ID NO: 343. | | |
| HPV113 | ... APSVVFHITDTSGDFYLHPSLRKRKRKRKYL SEQ. ID NO: 344. | | |
| MmuPV1 | ... PASTVISVDDDFADFYLHPSLRKRKRKYRIY SEQ. ID NO: 345. | | |
| EcPV2 | ... SVFVFTFSMNGLFDLHPSLHPRKRKRRYNCCFADGFLDNEQTTPVPTP | | |
| | QSRL SEQ. ID NO: 346. | | |
| HPV108 | ... PFVIVDSFISSDTFYLHPSLVRKRKRRDLV SEQ. ID NO: 347. | | |
| HPV137 | ... QPPDGVLVDDNDYYLHPGLYSRKRKRRVL SEQ. ID NO: 348. | | |
| HPV201 | ... TVPVLEYDFTSADFTLHPSLSRKRKRRPSF SEQ. ID NO: 349. | | |
| EsPV3 | ... DTPVITIDIWSSDFYLHPSLSRKRKRRRKFVFVY SEQ. ID NO: 350. | | |
| SaPV1 | ... IPTEPHKKGKPIHVHRKRRCIKRRGKRCVKYSL SEQ. ID NO: 351. | | |
| HPV74 | ... PTGPVFINGSTFYLYPSWYFARKRRKHVPLFFTDVAA | | |
| | SEQ. ID NO: 352. | | |
| HPV32 | ... PSFDSVMVLGWDFILHPSYMWRKRRKPVPYFFADVRVAA | | |
| | SEQ. ID NO: 353. | | |
| MfPV7 | ... PQGHITVLGGAFYWHPSWYTARKRRKLVPNFLADVSVAA | | |
| | SEQ. ID NO: 354. | | |
| HPV63 | ... IPLIIIHLDNSTGDYDLHPSLRKRRKLVHI SEQ. ID NO: 355. | | |
| ChPV1 | ... PIPAVLFNVLSSDLLLDPSLLRKRRKKYGVFS SEQ. ID NO: 356. | | |
| PhPV1 | ... PTARIIVYGGDFYLHPSYFGIRKRRKRVHHSFADVFVAA | | |
| | SEQ. ID NO: 357. | | |
| HPV7 | ... IPAISVLIRGTDYYLNPAYYFRKRRKRILAY SEQ. ID NO: 358. | | |
| HPV13 | ... PTGPVFITASGFYLYPTWYFTRKRRKRVSLFFTDVAA | | |
| | SEQ. ID NO: 359. | | |
| HPV16 | ... PQYTIIADAGDFYLHPSYYMLRKRRKRLPYFFSDVSLAA | | |
| | SEQ. ID NO: 360. | | |
| HPV40 | ... IPAISVLIHGTDYYLHPAYYLRKRRKRILAHQYVAT | | |
| | SEQ. ID NO: 361. | | |
| HPV44 | ... PTGPVFISGAAFYLYPTWYFARKRRKRVSLFFADVAA | | |
| | SEQ. ID NO: 362. | | |
| HPV51 | ... TKHSIVILGGDYYLWPYTHLLRKRRKRIPYFFTDGIVAH | | |
| | SEQ. ID NO: 363. | | |
| HPV54 | ... PQTPIAVNGGDFYLHPSYTYVRKRRKRFPYFLADGYVAA | | |
| | SEQ. ID NO: 364. | | |
| HPV82 | ... TQHAIVIQGGDYYLWPYTYLLRKRRKRIPYFFADGFVAY | | |
| | SEQ. ID NO: 365. | | |
| MfPV5 | ... PTSHVVVYGGDFYLHPSYYTIRKRRKRVHRFLSDVLVAA | | |
| | SEQ. ID NO: 366. | | |
| MfuPV1 | ... PSSHIVVYGGDFYLHPAYYPTRKRRKRMHRFLSDVLVAA | | |
| | SEQ. ID NO: 367. | | |
| PpPV1 | ... PITPIFISGSQFYLHPSLYLARKRRKRVSLFFADVAA | | |
| | SEQ. ID NO: 368. | | |
| SscPV3 | ... VVPSMSVTVFSGDYFLHPSYFRKRRKRVSYFFADGIVAA | | |
| | SEQ. ID NO: 369. | | |
| PIPV1 | ... PLIIIEVLDGSGDYFLHPSLFRKRRKRPFF SEQ. ID NO: 370. | | |
| HPV1 | ... TPLVIIALNNSTGDFELHPSLRKRRKRAYV SEQ. ID NO: 371. | | |
| HPV6 | ... PTGPVFITGSGFYLHPAWYFARKRRKRIPLFFSDVAA | | |
| | SEQ. ID NO: 372. | | |
| HPV118 | ... PVVVIHTHDTSGDFYLHPSLSRKRRKRKYL SEQ. ID NO: 373 | | |
| HPV124 | ... VWIHTHDATGDFYLHPSLTLRKRRKRKYL SEQ. ID NO: 374 | | |
| HPV146 | ... IGPSYDVAATNSDFIYDPDLFRKRRKRKLSAF SEQ. ID NO: 375 | | |
| HPV179 | ... LKPALGLYTLGEDFIFDPDLFRKRRKRKYSDV SEQ. ID NO: 376 | | |
| HPV152 | ... VVIIHTHDTTGDFYLHPSLTLRKRRKRKYL SEQ. ID NO: 377 | | |
| EhPV2 | ... AGPSIDVGDVGIDYFLDPYLFRKRRKRKRFFSFADDHVDS | | |
| | SEQ. ID NO: 378 | | |
| CPV13 | ... PTVDLSDFELSMTFDLHPSLLRKRRKRKRTFL SEQ. ID NO: 379 | | |
| AmPV3 | ... RPPALGGFPGTDYYLHPGLSHRKRRKRRPFWFLL SEQ. ID NO: 380 | | |
| HPV131 | ... VEPSLIITDSHDFELHPALWPRKRRRLDLF SEQ. ID NO: 381 | | |
| HPV157 | ... ILPQYVIETFHDFFLTPDLYPRKRRRIDFF SEQ. ID NO: 382 | | |
| HPV200 | ... PWYSIIERNFADFVLDPAFIPRKRRRLEIL SEQ. ID NO: 383 | | |
| PaPV1 | ... PGVLISYDGSMDPSLYYLLFYRKRRRLHRLFYR SEQ. ID NO: 384 | | |
| PaPV2 | ... NPGIPIAYYDNLDPSLIWWYLRKRRRLQHLFYR SEQ. ID NO: 385 | | |
| HPV29 | ... THYVYIDGGDYFLWPVTFPVSRKRRRKRLSYFLADGFVAL | | |
| | SEQ. ID NO: 386 | | |
| HPV77 | ... THYVYIDGGNFYLWPVTFSVSRKRRRKRLSYFFADGTVAL | | |
| | SEQ. ID NO: 387 | | |
| HPV149 | ... IPSILIDGFSSDDFVLHPSHSRKRRRKRTPLL SEQ. ID NO: 388 | | |
| HPV99 | ... IPVVVIHTHDYSGDFYLHPSLRKRRRKRKYL SEQ. ID NO: 389 | | |
| HPV110 | ... TVVIHISDTSGDYYLHPSLQTRKRRRKRKYL SEQ. ID NO: 390 | | |
| CPV8 | ... PPTVVVGSFGGVDYSLHPSLLRKRRRRRKRYIS SEQ. ID NO: 391 | | |
| CPV5 | ... LVIADITMGEGTDYYLHPSLTRKRRRRRRRRSL SEQ. ID NO: 392 | | |
| MfPV2 | ... PTVVIRTSDSSGDFYLHPSLLRLKRKRKYL SEQ. ID NO: 393 | | |
| HPV173 | ... EPLPPAIVELDNFDYDLHPSLRLKRKRKRTDLV SEQ. ID NO: 394 | | |
| RfPV1 | ... NDTPAVYIDPFSADYYLHPSLRLKRRRRRGQFVYVY | | |
| | SEQ. ID NO: 395 | | |
| PpuPV1 | ... TPEIIVTFLDSSYTVADPSLFRLRKRRKRRFH SEQ. ID NO: 396 | | |
| TtPV6 | ... PFVVLHFSGGGTSFFLHPSLLRLRRKRVFY SEQ. ID NO: 397 | | |
| TmPV4 | ... IMFPQASHISSFDFLLHPSALRLRRKRKRSVHDDDGTIIE | | |
| | SEQ. ID NO: 398 | | |
| HPV205 | ... PLPPTFVDFDNFDYDIHPGLLRRKKRKRTDLV SEQ. ID NO: 399 | | |
| PphPV2 | ... KPTVTFDALDIGGGFYLHPYIRRKKRKRMYL SEQ. ID NO: 400 | | |
| VvPV1 | ... LQPVIVVSPFESFDYNLHPSLRRKKRKRPYFF SEQ. ID NO: 401 | | |
| HPV22 | ... GPAIIQSPTHSSFDYYLHPSLRRKKRKRKYL SEQ. ID NO: 402 | | |
| HPV116 | ... LTPSFSIDVNYSDYNIHPANIRRKRKHSSSLYF SEQ. ID NO: 403 | | |
| HPV41 | ... PYIVVDLYSGSMDYDIHPSLLRRKRKKRKRVYFSDGRVASRPK | | |
| | SEQ. ID NO: 404 | | |
| CPV7 | ... SNASDTTSDFISVTFDLHPSLRRKRKKRKRRYI SEQ. ID NO: 405 | | |
| MfPV8 | ... PHGHVVVFGGDFLWHPSWYSPRRKRKRLPTFFADVSVAA | | |
| | SEQ. ID NO: 406 | | |
| MfPV11 | ... PQGHVYVSGGDFLWHPSLYTPRRKRKRVHTFFADVSVAA | | |
| | SEQ. ID NO: 407 | | |
| MmPV1 | ... PSAHIILYGGDFYLHPSYLGIRRKRKRMHNFFSDVYVAA | | |
| | SEQ. ID NO: 408 | | |
| HPV209 | ... PAVVIHFGEPGGDFYLHPDLQRRKRKRAYL SEQ. ID NO: 409 | | |
| OaPV1 | ... DGNIIIYSTYFKHYYLHPSLYRRKRKRLLD SEQ. ID NO: 410 | | |
| OvPV1 | ... DGSIALSLEYFRHYYLHPSLLRRKRKRNPIFI SEQ. ID NO: 411 | | |
| RtPV1 | ... DGDISLSVEYFRHYYLHPSLLRRKRKRLFN SEQ. ID NO: 412 | | |
| EcPV6 | ... PGTPEVVIDIYPHTPLAFLHHRRKRKRGSSVFFADVLLAF | | |
| | SEQ. ID NO: 413 | | |
| HPV109 | ... PSILINESSSDDFVLHPSHIPRRKRKRAYPF SEQ. ID NO: 414 | | |
| HPV154 | ... VVPGFDINVEASDFNIHPSVLRRKRKRSMF SEQ. ID NO: 415 | | |
| PsPV1 | ... PSILLFYPDSSPSFYLHPSLLRRKRKRVFY SEQ. ID NO: 416 | | |
| HPV138 | ... PSILIDEFSSDDFVLHPSHIPRRKRKRIDSL SEQ. ID NO: 417 | | |
| HPV107 | ... PAVVIHFGESGADYYLHPHLQRRKRKRAYL SEQ. ID NO: 418 | | |
| OaPV4 | ... DGNIIIYSTYLRHYYLHPSLYRRKRKRLLD SEQ. ID NO: 419 | | |
| HPV5 | ... PVVIIHPHDSTGDFYLHPSLHRRKRKRKYL SEQ. ID NO: 420 | | |
| BPV21 | ... DDPDITLAIFGTDFYLHPGLLRRKRKRKNFSV SEQ. ID NO: 421 | | |
| HPV101 | ... EPPIVVDGFDAFDTFYLHPSHRRKRKRKRSGF SEQ. ID NO: 422 | | |
| HPV155 | ... PSILIDGFSSDDFVLHPSHIPRRKRKRKRSL SEQ. ID NO: 423 | | |
| EdPV1 | ... DDTLDTFQNYNDYDLHPSLLPRRKRKRRIL SEQ. ID NO: 424 | | |
| EcPV5 | ... DVIIEYPEAGGSYFLHPTAPCRRKRRYCFADGLLDAGQSEVLSPPC | | |
| | ACY SEQ. ID NO: 425 | | |
| UmPV1 | ... TTPHAGVYVSVDFWLHPGLLSRRKRRFPFLFTDGIVAA | | |
| | SEQ. ID NO: 426 | | |
| BPV17 | ... PPYIIDFNDNSATFYLHPSLIRRKRRKRKRIFS SEQ. ID NO: 427 | | |
| CePV2 | ... PVIIDFGTSGATFYLHPSLFLRRKRRKRRFL SEQ. ID NO: 428 | | |
| RnPV3 | ... HPPIYVGSVPGVDYYLHPSLSRRKRRRRHSYL SEQ. ID NO: 429 | | |
| CPV12 | ... PIIIVDVAVGAGTDYYLHPSLRRKRRRRGRRHFM SEQ. ID NO: 430 | | |
| CdPV2 | ... GIIIDLSEELFKHYYLHESLLRRKRRRRKRLYA SEQ. ID NO: 431 | | |
| EsPV1 | ... SDTPIVTLDIWSSDFYLHPSLRRKRRRRRKYVFVY SEQ. ID NO: 432 | | |
| CPV10 | ... PRPDVVIYYYGGVDYSLHPSLRRKRRRRRRKRVSF SEQ. ID NO: 433 | | |
| MfPV1 | ... PIVVIHPHDNSGDFYLHPSLTRRLRRKRKYL SEQ. ID NO: 434 | | |
| BPV20 | ... PDIILDIYTPGSTFYLHPSHYRRRGRKRKRTVF SEQ. ID NO: 435 | | |
| SsPV2 | ... PLVIIDVLGSGDFLLHPSLLQRRRKRIKSVF SEQ. ID NO: 436 | | |
| RnPV1 | ... PGTEIVFIPEDSIDFYLHPSLRRRKRKYFVR SEQ. ID NO: 437 | | |
| HPV49 | ... LPTVIIHTADTSGDFYLHPSLRRRKRKRTYL SEQ. ID NO: 438 | | |
| HPV75 | ... LPAVVIHTSDTSGDFYLHPSLRRRKRKRAYL SEQ. ID NO: 439 | | |
| HPV76 | ... LPAVVIHTSDTSGDFYLHPSLRRRKRKRAYL SEQ. ID NO: 440 | | |
| HPV96 | ... TPTIIIHTEDFSGDYYLHPSLRRRKRKRAYL SEQ. ID NO: 441 | | |
| SsPV1 | ... RPGVGLVDNAFMFAWGPQYILRRRKRKRPLPPLFADGFVAA | | |
| | SEQ. ID NO: 442 | | |
| HPV60 | ... IIQPAMAVDVYDDFYLHPHLLRRRKRKRLDFF SEQ. ID NO: 443 | | |
| HPV126 | ... PIGPSAALDVEYYDFDLHPSLRRRKRKRNMF SEQ. ID NO: 444 | | |
| HPV139 | ... TPSILIDEFSSDDFILHPSHIRRRKRKRLNSL SEQ. ID NO: 445 | | |
| HPV141 | ... PLQPPYEAEVVFDTFDLHPSLRRRKRKRSSF SEQ. ID NO: 446 | | |
| HPV163 | ... PYAPPLLYDFESHDFILHPSHRRRKRKRIAMF SEQ. ID NO: 447 | | |
| MaPV1 | ... GVDINVGSIYPSVDYYLHPSLRRRKRKRTLH SEQ. ID NO: 448 | | |
| PsuPV1 | ... GTTHIVGSEFSSVDYYLHPSLRRRKRKRNFH SEQ. ID NO: 449 | | |
| HPV8 | ... LPVVIIHTHDNSGDFFLHPSLRRRKRKRKYL SEQ. ID NO: 450 | | |
| HPV12 | ... IPVVVIHTHDNSGDFYLHPSLRRRKRKRKYL SEQ. ID NO: 451 | | |
| HPV47 | ... LPVVVIHTHDNSGDFYLHPSLRRRKRKRKYL SEQ. ID NO: 452 | | |
| HPV151 | ... GPAIVQSLTHTSLDYYLHPSLRRRKRKRKYL SEQ. ID NO: 453 | | |
| EcPV1 | ... IIILVDSPDTSGVFDLHPSLLRRRKRRYMWN SEQ. ID NO: 454 | | |
| ZcPV1 | ... VPPLPGYSNYGGPDFYLHPSLRRRKRRKRRYLSSFVF | | |
| | SEQ. ID NO: 455 | | |
| MnPV1 | ... RPHVIYRGYNGTDYYLHPSLSRRRRNSRHIYFSDGVLAA | | |
| | SEQ. ID NO: 456 | | |
| FIPV1 | ... PGVLVGYNDFAKDPSLYWWFIRRRRARRFHPYSRSR | | |
| | SEQ. ID NO: 457 | | |
| HPV42 | ... PQGNFVMVSGWDFILHPSYFWRRRRKPVPYFFADVRVAA | | |
| | SEQ. ID NO: 458 | | |
| MfPV4 | ... PSSHVVVYGGDFYLHPSYYPVRRRRKHMPRFLSDVVVAA | | |
| | SEQ. ID NO: 459 | | |
| CcanPV1 | ... MIPWIIIGTQGSDYDLHPSLLRRRRKLSFL SEQ. ID NO: 460 | | |
| HPV11 | ... PTGPVFITGSDFYLHPTWYFARRRRKRIPLFFTDVAA | | |
| | SEQ. ID NO: 461 | | |
| OaPV2 | ... DGNIVIYSTYLKHYYLHPSLYRRRRKRLLD SEQ. ID NO: 462 | | |
| HPV33 | ... PFDTIVVDGADFVLHPSYFILRRRRKRFPYFFTDVRVAA | | |
| | SEQ. ID NO: 463 | | |
| HPV52 | ... PSTSIIVDGTDFILHPSYFLLRRRRKRFPYFFTDVRVAA | | |
| | SEQ. ID NO: 464 | | |
| HPV58 | .. PFNTIIVDGADFMLHPSYFILRRRRKRFPYFFADVRVAA | | |
| | SEQ. ID NO: 465 | | |
| HPV67 | ... SPFNNVLVYGSDFILHPSYFLRRRRKRFPYFFADVRVAA | | |
| | SEQ. ID NO: 466 | | |
| HPV78 | ... HYVYVDGGDFYLWPITFYVSHRRRRKRVSYFFTDGTVAT | | |
| | SEQ. ID NO: 467 | | |
| HPV160 | ... QYVYIDGGDFYLWPVTFSLFGRRRRKRLSYFFADGTVAL | | |
| | SEQ. ID NO: 468 | | |
| MfPV3 | ... PSADIIVNGGDFYLHPSYLSLRRRRKRMHRFFADVLVAA | | |
| | SEQ. ID NO: 469 | | |
| MfPV10 | ... PTAHIVVHGGDFYLHPSYLYVRRRRKRLHHFFTDVYVAA | | |
| | SEQ. ID NO: 470 | | |
| EcPV3 | ... GGDREVVVNMYPYSIMSFLHYRRRRKRGYVYFSDVILAI | | |
| | SEQ. ID NO: 471 | | |
| EcPV7 | ... PEFPEIVIDGYGLSALTFVLHRRRRKRGSHLYFSDVLLAV | | |
| | SEQ. ID NO: 472 | | |
| BPV12 | ... PAIIIDFTDSTATFYLHPSLMRRRRKRHIF SEQ. ID NO: 473 | | |
| HPV24 | ... AVVIHTEDSSGDFYLHPSLLQRRRRKRKYL SEQ. ID NO: 474 | | |
| HPV25 | ... WVIIHTYDTSGDFYLHPSLTTRRRRKRKYL SEQ. ID NO: 475 | | |
| HPV145 | ... VVIHVEDSSGDYYLHPSLRVTRRRRKRKYL SEQ. ID NO: 476 | | |
| HPV167 | ... DAPLVTINALGSDYDLHPSYLRRRRKRKYSEIF SEQ. ID NO: 477 | | |
| OcPV1 | ... GTITPSFSFNNSGDFVLHPSLRRRRKRKFVF SEQ. ID NO: 478 | | |
| EsPV2 | ... DIIIHDVGSSVDYILHPGLLPRRRRKRKRSY SEQ. ID NO: 479 | | |
| HPV88 | ... SEPDIRLDWFSPDYDLHPSLLRRRRKRKRNMF SEQ. ID NO: 480 | | |
| HPV178 | ... PLEPPIILDFQSSTYYLHPSLRRRRKRKRSDIYDFLSDGSVDT | | |
| | PEW SEQ. ID NO: 481 | | |
| BPV19 | ... GPDLIINLYGNDYSLHPSYLLRRRRKRKRFHVL SEQ. ID NO: 482 | | |
| CPV19 | ... DAPDLTSSEYLSVTFDLHPSLRRRRKRKRRYI SEQ. ID NO: 483 | | |
| DdPV1 | ... PSVVILSTDDFGDMFRFWHLLRRRRKRRYL SEQ. ID NO: 484 | | |
| TtPV1 | ... PDVVIYSTDDFGYMFRFWHFLRRRRKRRYL SEQ. ID NO: 485 | | |
| TtPV4 | ... PHVVIYYTDDYGDMFRFWHLLRRRRKRRYL SEQ. ID NO: 486 | | |
| TtPV7 | ... PHVNIYYTDEFGDMFRFWHFLRRRRKRRYL SEQ. ID NO: 487 | | |
| FcaPV2 | ... RPAPSDDTVYYSAGVLDPSLLRRRRKRRRSVAYGF | | |
| | SEQ. ID NO: 488 | | |
| HPV30 | ... DTTHDVVIHGSTFALWPVYFLRRRRRKHVPYFLADGGVAA | | |
| | SEQ. ID NO: 489 | | |
| HPV3 | ... THYVYIDGGDFYLWPVTFFLPRRRRRKRVSYFLADGTVAL | | |
| | SEQ. ID NO: 490 | | |
| HPV28 | ... THYVYIDGGDFYLWPVTLFVPRRRRRKRLSYFLADGTVAL | | |
| | SEQ. ID NO: 491 | | |
| HPV56 | ... DVTHDVYIQGSSFALWPVYFFRRRRRKRIPYFFADGDVAA | | |
| | SEQ. ID NO: 492 | | |
| HPV94 | ... THYVYIDGGDFYLWPVTFYLSRRRRRKRV SEQ. ID NO: 493 | | |
| HPV117 | ... THYVYINGGDFYLWPVTFPLSRRRRRKRVSYFFTDGTLAP | | |
| | SEQ. ID NO: 494 | | |
| HPV125 | ... THYVYIDGADFYLWPVALFVPRRRRRKRISYFLADGTVAL | | |
| | SEQ. ID NO: 495 | | |
| HPV93 | ... PAVVIHTYDNSGDFYLHPSLLRRRRRKRKYL SEQ. ID NO: 496 | | |
| HPV98 | ... PVVVIHTHDNSGDFYLHPSLFRRRRRKRKYL SEQ. ID NO: 497 | | |
| CPV15 | ... PRVVVFYYGGVDYSLHPSLLFRRRRRKRRRHAF SEQ. ID NO: 498 | | |
| FcPV1 | ... FPSGVDINVTDPSLYWLRWWLRRRRRRGYLLFR SEQ. ID NO: 499 | | |
| CPV14 | ... PTVVVGYFGGVDYSLHPSLMFRRRRRRKRYIS SEQ. ID NO: 500 | | |
| TtPV3 | ... SDWVIYSTDDFGDMFRFWHFLRRRRRRRYL SEQ. ID NO: 501 | | |
| CPV9 | ... IIVDVASGAGTDYYLHPSLQARRRRRRRRHFM SEQ. ID NO: 502 | | |
| PePV1 | .. APVSYGGLVSMDPNSLFWFLLRRRRRRRRTTKRILLNR | | |
| | SEQ. ID NO: 503 | | |
| CPV18 | ... IIVDVERGSGSDYYLHPSLTLRRRRRRRRKSL SEQ. ID NO: 504 | | |
| CPV4 | ... IVDVARGPSSDYFLHPSLYATRRRRRRRRRKHI SEQ. ID NO: 505 | | |
| CPV11 | ... IIIVDVIGASGSDYYLHPALSRRRRRRRRRRSL SEQ. ID NO: 506 | | |
| CPV16 | ... VIIVDVSVGSSTDYYLHPSLARRRRRRRRRRSL SEQ. ID NO: 507 | | |
| CPV20 | ... IIIVDIIGGAGSDYYLHPSLSRRRRRRRRRRSQ SEQ. ID NO: 508 | | |
| HPV174 | ... APAVVIHVEDSSGDFYLHPSLRSKKRKRKYL SEQ. ID NO: 509 | | |
| BPV18 | ... PSSLQPTNYFDYWQLFEPSLWRSRKRKRNVYY SEQ. ID NO: 510 | | |
| HPV120 | ... IPAVIVHIIDTSFDYYLHPSLRTKRRKRKYL SEQ. ID NO: 511 | | |
| FgPV1 | ... PYWPGEGWPPPHFEFSDPSLYRVKKRRWDDCIAIMITVT | | |
| | SEQ. ID NO: 512 | | |
| HPV100 | ... VPAVIVHITDTSGDFYLHPSLRWKKRKRKYL SEQ. ID NO: 513 | | |
| HPV143 | ... TPVVVIHTEDNTGDFYLHPSLRWRKRKRHYL SEQ. ID NO: 514 | | |
| HPV36 | ... IPVVVIHTHDNTGDFYLHPSLRWRKRKRKYL SEQ. ID NO: 515 | | |
| FcaPV3 | ... GQPSLLWDPSTGTFDLHPGLLRWRRKRRRRHDL SEQ. ID NO: 516 | | |
| HPV38 | ... IPTVVIHVADSSGDFYLHPSLRWRRRKRKYL SEQ. ID NO: 517 | | |

### Methods of Preventing Viral Infection

Without wishing to be limited by theory, it has now been found that delivering a peptide containing a retromer binding site to a cell in advance of or shortly after exposure to a virus prevents viral infection by sequestering retromer and interfering with the role this complex plays in the viral infection of the cell. Accordingly, in another aspect, the invention provides a method of preventing viral infection in a subject in need thereof, the method comprising providing to the subject a therapeutically effective amount of a polypeptide comprising a cell-penetrating peptide and a retromer binding site. In various embodiments, the viral infection is papillomavirus, hepatitis C virus, influenza virus or human immunodeficiency virus (HIV). The polypeptide is provided to the subject by a method appropriate for the viral infection to be prevented. In various embodiments, the polypeptide is formulated for topical administration.

The cell-penetrating peptide may be any cell-penetrating peptide known in the art including but not limited to the cell-penetrating peptides taught herein. In various embodiments, the polypeptide comprises at least one cell-penetrating peptide selected from the group consisting of SEQ ID NOS: 1-164. In various embodiments, the polypeptide comprises at least one cell-penetrating peptide selected from the group consisting of SEQ ID NOS: 165-517. In various embodiments, the polypeptide comprises a retromer binding site comprising the sequence FYL.

### Administration/Dosage/Formulations

The regimen of administration can affect what constitutes an effective amount for the treatment of various diseases, depending on the nature of the cargo molecule. The therapeutic formulations can be administered to the subject either prior to or after the onset of diseases contemplated herein. Further, several divided dosages, as well as staggered dosages can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

Administration of the compositions of the present invention to a patient, preferably a mammal, more preferably a human, can be carried out using known procedures, at dosages and for periods of time effective to treat the disease in the patient. An effective amount of the therapeutic compound necessary to achieve a therapeutic effect can vary according to factors such as the state of the disease or disorder in the patient; the age, sex, and weight of the patient; and the ability of the therapeutic compound to treat the disease in the patient. Dosage regimens can be adjusted to provide the optimum therapeutic response. For example, several divided doses can be administered daily or the dose can be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention can be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of an inflammatory disease in a patient.

The carrier can be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration can be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of the disease in a patient.

Formulations can be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents.

Routes of administration of any of the compositions of the invention include oral, nasal, rectal, intravaginal, parenteral, buccal, sublingual or topical. The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use can be prepared according to any method known in the art and such compositions can contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets can be uncoated or they can be coated by known techniques for elegance or to delay the release of the active ingredients. Formulations for oral use can also be presented as hard gelatin capsules wherein the active ingredient is mixed with an inert diluent.

The present invention also includes a multi-layer tablet comprising a layer providing for the delayed release of one or more compounds of the invention, and a further layer providing for the immediate release of a medication for treatment of certain diseases or disorders. Using a wax/pH-sensitive polymer mix, a gastric insoluble composition can be obtained in which the active ingredient is entrapped, ensuring its delayed release.

### Parenteral Administration

For parenteral administration, the compounds of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of an inflammatory disease in the patient being treated. The skilled artisan is able to determine appropriate dosages depending on these and other factors.

A suitable dose of a compound of the present invention can be in the range of from about 0.01 mg to about 5,000 mg per day, such as from about 0.1 mg to about 1,000 mg, for example, from about 1 mg to about 500 mg, such as about 5 mg to about 250 mg per day. The dose can be administered in a single dosage or in multiple dosages, for example from 1 to 4 or more times per day. When multiple dosages are used, the amount of each dosage can be the same or different. For example, a dose of 1 mg per day can be administered as two 0.5 mg doses, with about a 12-hour interval between doses.

It is understood that the amount of compound dosed per day can be administered, in non-limiting examples, every day, every other day, every 2 days, every 3 days, every 4 days, or every 5 days. For example, with every other day administration, a 5 mg per day dose can be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in practicing the following examples 1-9 are here described:

### Cells, Plasmids, and Virus

HeLa S3 cervical carcinoma cells were obtained from the American Type Culture Collection (ATCC). HeLa-Sen2, a cloned strain of HeLa cells, infect efficiently with SV40 and HPV16 pseudovirus and were used for immunofluorescence experiments. HaCaT adult human keratinocyte cells were purchased from AddexBio. The HPV16 L2 C-terminal mutants were constructed in pHPV16sheLL by using the Phusion site-directed mutagenesis protocol (New England Biolabs, Ipswich, MA). This method was also used to replace the basic segment of the L2 protein with known CPPs: cationic (SEQ ID NO: 519 RKKRRRQRRR), amphipathic (SEQ ID NO: 520 PLSSIFSRIDG), and hydrophobic (SEQ ID NO: 521 AAVLLPVLLAAP), from HIV Tat, hepatitis B virus large surface antigen, and the K-fibroblast growth factor signal peptide, respectively. The L1 and L2 genes in each mutant were sequenced to verify mutations.

HPV PsVs were produced by co-transfecting 293TT cells with pCAG-HcRed (cat. #11152; Addgene, Cambridge, MA) and p16sheLL or p16L1-GFP with or without mutations or a FLAG tag at the C-terminus of L2. The packaged PsVs were purified by density gradient centrifugation in Opti Prep (cat. ##1114524; Axis-shield, Dundee, Scotland). Encapsidated HcRed genomes were quantified by qPCR as described. Briefly, purified PsVs were treated with DNase I (cat. #79254; Qiagen, Germantown, MD) to remove unencapsidated DNA associated with capsids. Reporter DNA genome was isolated using a DNA purification kit (Qiagen, #69504), and the copy number of encapsidated reporter plasmids was determined by qPCR using primers for the HcRed gene in comparison to a standard curve.

### Characterization of Purified Pseudovirus

Five μl of pseudovirus solution was placed on freshly glow-discharged copper grids (formvar/carbon-coated, 200 mesh, Electron Microscopy Services, Hatfield, PA). After two min, the grids were rinsed twice with droplets of deionized water, stained by 2% aqueous uranyl acetate for two min, and then the excess staining solution was blotted off. The grids were allowed to air dry for 15 min. The samples were examined in a FEI Tecnai F20 transmission electron microscope at 200 kV. Images were acquired using a FEI Eagle CCD camera (4K×4K). Virus stocks containing ˜1×107 PsV genomes were analyzed by SDS-PAGE, followed by staining with Coomassie brilliant blue.

### Infectivity

5×10⁴ HeLa S3 or HaCaT cells in 12-well plates were incubated with PsVs at MOI of approximately one. The number of packaged reporter plasmids required to achieve this MOI as assessed by flow cytometry for reporter gene expression from wild-type PsV was quantified by qPCR, and an equivalent number of genomes in mutant PsV were used to infect cells. Approximately five-fold more virus was used to attain this MOI in HaCaT cells. Cells were analyzed by flow cytometry on a Stratedigm-12 flow cytometer to determine the fraction of cells displaying HcRed fluorescence 48 h.p.i. To measure dependence on retromer, 1×105 HeLa S3 cells in a 12-well plate were transfected 48 h.p.i. prior to infection with 10 pmol siRNAs targeting Vps29 (Dharmacon, Lafayette, CO; #D-009764-03) or Vps35 (Dharmacon, #D-010894-01) by using RNAiMAX (Thermo Fisher, Waltham, MA, #13778100) according to manufacturer's protocol. Cells transfected with a 10 pmol RISC-free siRNA were used as a control. Forty-eight h.p.i., infectivity was measured by flow cytometry for reporter gene expression. To measure dependence on γ-secretase, HeLa S3 cells were pretreated for one h with 250 nM γ-secretase inhibitor, compound XXI, which was retained in the medium for the duration of the experiment. Cells were infected at MOI of one. Forty-eight h.p.i., infectivity was measured by flow cytometry for HcRed expression. To measure the effect of aphidicolin, HeLa S3 cells were pretreated for 24 h with 6 μM aphidicolin, which was retained in the medium for the duration of the experiment. Cells were infected at MOI of one. Forty-eight h.p.i., infectivity was measured by flow cytometry for HcRed expression.

### Immunofluorescence Microscopy and Proximity Ligation Assay

To measure cell binding, 5×10⁴ HeLa-Sen2 cells grown on glass coverslips were infected with wild-type PsV at MOI of 20 or mutant PsV containing the same number of encapsidated reporter plasmids. Cells were incubated with PsVs at 4° C. for two h and then washed three times with phosphate-buffered saline (PBS). Cells were fixed for 15 min at room temperature with 4% Formalde-Fresh, washed with PBS and reacted with anti-L1 rabbit polyclonal serum (obtained from J. Schiller, NIH), followed by fluorescently-tagged donkey anti-rabbit secondary antibody. Images were acquired with a Leica SP5 inverted fluorescence microscope and processed with ImageJ.

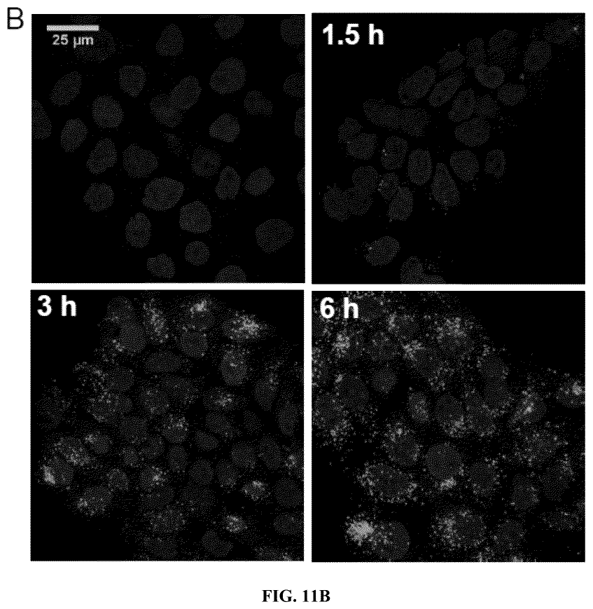

For the internalization experiments, HeLa cells were incubated with PsV at MOI of 50 at 4° C. for two h, followed by extensive washing in PBS to remove loosely bound PsV. Cells were then shifted to 37° C. to initiate internalization. After six hours, samples were fixed, permeabilized and reacted with anti-L1 antibody (BD Biosciences, San Jose, CA, #554171), followed by donkey anti-rabbit secondary antibody. Cells were analyzed by fluorescence confocal microscopy. In the experiment shown in FIG. 11B, cells were infected with wild-type HPV16 PsV and stained with the antibody 33L1-7, which recognizes L1 after virus is internalized and capsid disassembly has begun.

For the proximity ligation assay, HeLa-Sen2 cells were infected with wild-type PsV at MOI of 100-to-200 or mutant PsV containing the same number of encapsidated reporter plasmids. Infected cells were fixed, permeabilized at various times post-infection, and incubated with pairs of antibodies, one recognizing L1 (#554171) and the other recognizing a cellular marker or a retromer subunit. PLA was performed with Duolink reagents from Olink Biosciences (Uppsala, Sweden) according to the manufacturer's directions. Briefly, cells were incubated in a humidified chamber with a pair of suitable PLA antibody probes diluted 1:5 and processed for ligation and amplification with fluorescent substrate at 37° C. Images were acquired as described above. Approximately 100 nuclei in each sample were imaged. The images were processed by ImageJ and quantitatively analyzed by BlobFinder software to measure total fluorescence intensity in each sample. The average fluorescence intensity per cell in each sample was normalized to the control sample as indicated in each experiment. Similar results were obtained in three independent experiments for each antibody pair.

In all fluorescence imaging experiments, cells were also stained with DAPI or Hoechst 33342 to visualize nuclei (blue), and a single confocal Z-plane is shown in each panel.

### Flow Cytometry to Measure Binding and Internalization

To measure cell surface binding by wild-type and mutant PsV, 7.5×10⁵ HeLa cells in six-well plates were incubated with wild-type PsVs at MOI of 20 or mutant PsVs containing the same number of reporter plasmids at 4° C. for two h and then washed three times with PBS. Cells were harvested by treatment of 0.5 mM EDTA on ice for 15 min. Samples were fixed in ice-cold methanol and stained with mouse anti-L1 polyclonal IgG (BD Biosciences, #554171) and incubated with corresponding Alexa Fluor secondary antibodies. Fluorescence intensity was assayed on a Stratedigm-13 flow cytometer.

To measure internalization, 7.5×10⁵ HeLa cells in six-well plates were incubated at 4° C. for two h with wild-type PsV at MOI of 20 or mutant PsV containing the same number of reporter plasmids and then washed three times with PBS, followed by additional incubation at 37° C. for six h to initiate internalization. Cells were then harvested by trypsinization to remove PsV on the cell surface. Samples were fixed in ice-cold methanol and stained with anti-L1 antibody #554171 and incubated with Alexa Fluor-labeled secondary antibody. Fluorescence intensity was assayed on a Stratedigm-13 flow cytometer.

### Peptide and Fusion Protein Cellular Uptake Experiments

5×10⁴ 293T cells grown on eight-chambered glass coverslips were incubated with 30 μM fluorescently-labeled peptides for three hours at 37° C. or mock-infected. 5×10⁴ HaCaT and HeLa S3 cells grown on eight-chambered glass coverslips were incubated with 2.5 μM wild-type or mutant His-tagged GFP-L2 fusion proteins (see next section) for three h at 37° C. After treatment, cells were washed with PBS three times, stained with Hoechst 33342, and examined by confocal microscopy. To observe internalized fusion proteins, cells were treated for 10-15 min with 0.04% trypan blue, washed with PBS, and examined by confocal microscopy.

### Fusion Protein and Peptide Pull-Down Experiments

For fusion protein pull-down experiments, individual human Vps26, Vps29, and GST-tagged Vps35 subunits were expressed individually in E. coli, and the assembled trimeric retromer complex was immobilized on GSH resin (GE Health Care Life Sciences, Pittsburgh, PA, #17075601). His6-GFP-L2 fusion proteins containing a C-terminal segment from wild-type or mutant HPV16 L2 (amino acids 435-461) were expressed in bacteria and purified using the His GraviTrap column (GE Healthcare, #11-0033-99). Purified proteins were exchanged into PBS buffer by dialysis and quantified by bicinchoninic acid protein assay. Ten g fusion protein was incubated with assembled 15 μg retromer trimer immobilized on GSH resin for two h at 4° C. in 20 mM HEPES pH 8.0, 50 mM NaCl, 5 mM MgCl₂, 1 mM DTT, and 0.1% Triton X-100. Beads were centrifuged and washed twice in HEPES buffer, suspended in SDS loading buffer, boiled, and subjected to SDS-PAGE and anti-GFP immunoblotting.

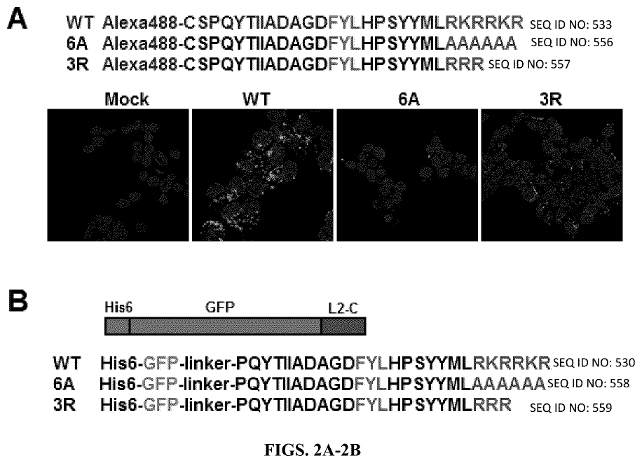

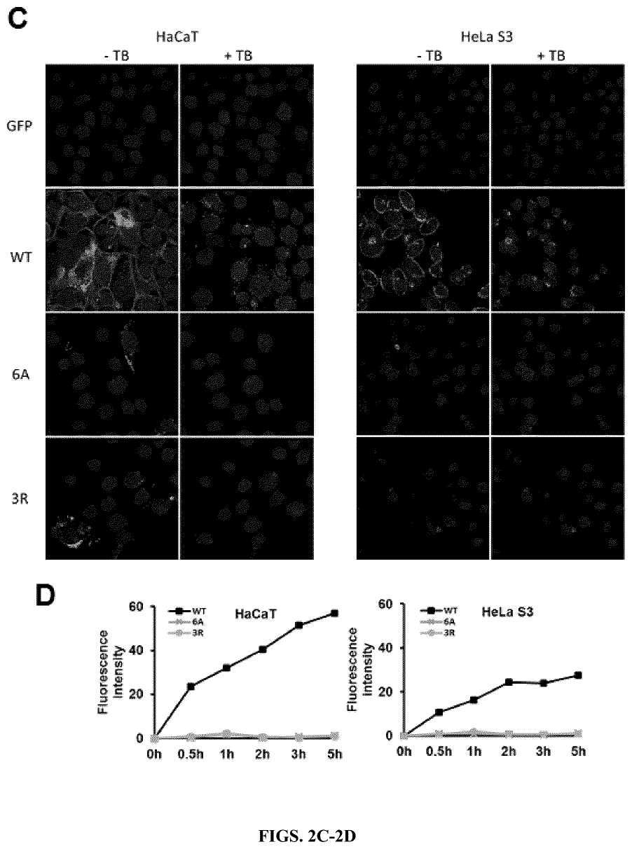

For peptide pull-down experiments, N-terminal biotinylated L2 peptides (sequences shown in FIG. 2A) were purchased from ABClonal Science (Woburn, MA) at >95% purity. L2 wild-type was dissolved initially in 30% acetic acid and DMSO (˜70 μl each), and then dissolved in sterile deionized water with 0.01% sodium azide. L2-6A, L2-3R and L2-DM were initially solubilized in a small amount of DMSO (˜70-80 l) and then dissolved in sterile deionized water with 0.01% sodium azide. Peptide stocks (5 mg/ml) were aliquoted and stored at −20° C. HeLa S3 cells plated in six-well plates were lysed at ˜80% confluency with 500 μl RIPA-MOPS buffer (20 mM morpholinepropanesulfonic acid [pH 7.0], 150 mM NaCl, 1% Nonidet P-40, 1 mM EDTA, 1% deoxycholic acid, 0.1% sodium dodecyl sulfate [SDS]) supplemented with protease inhibitors (1×HALT protease and phosphatase inhibitor cocktail (Thermo Scientific, Waltham, MA; #78443)). The lysate was centrifuged at 14,000 rpm for 20 min, and the supernatant was incubated with 10 μg of a biotinylated peptide for two h at 4° C. Fifty μl of streptavidin agarose beads slurry (Pierce, Rockford, IL; #20349) was added, and the mixture was gently rocked for 45 min at 4° C. Beads were recovered by centrifugation and washed four times with RIPA-MOPS buffer supplemented with NaCl to a final concentration of 0.4M. Samples were analyzed by SDS-PAGE and immunoblotting with antibody recognizing Vps35 (Abcam, Cambridge, MA; ab57632).

### Split GFP Assay

Plasmids. The full-length GFP gene in pLenti CMV GFP Puro (658-5) (Addgene, #17448) contained on a BamHI and SalI fragment was replaced with the DNA segment encoding GFP1-10 amplified from pCMV-mGFP1-10 to generate pLenti CMV.GFP1-10. To construct pLentiCMV.GFP1-10NES, oligonucleotides encoding the nuclear export signal (NES) sequence LPPLERLTLD (SEQ ID NO: 522) were inserted in-frame at the C-terminus of GFP1-10 in pLentiCMV.GFP1-10.

Plasmid pCD8-CIMPR expresses a CD8-CIMPR fusion protein containing the extracellular domain of CD8 fused to the transmembrane and mutant cytoplasmic domain of the cation-independent mannose phosphate receptor (CIMPR), which contains an endocytosis motif and three alanines replacing the WLM retromer binding site. The In-Fusion HD cloning Kit (TaKaRa Bio USA, Inc., Kusatsu, Shiga, Japan), #121416) was used to insert seven tandem copies of GFP11 (SEQ ID NO: 523 RDHMVLHEYVNAAGIT) in-frame into pCD8-CIMPR either immediately downstream of the CD8 signal peptide sequence or at the C-terminus of the fusion protein to generate pGFP11-CD8-CIMPR and pCD8-CIMPR-GFP11, respectively.

Each GFP 11 repeat was separated by short GGSGG (SEQ ID NO: 524)linker sequences. The sequences of the relevant portions of the encoded CD8-CIMPR fusion proteins are as follows, with GFP11 and linker sequences in bold.

GFP11-CD8-CIMPR,
(SEQ ID NO: 525)
MALPVTALLLPLALLLHAARP(RDHMVLHEYVNAAGITGGSGG)₆RDH
MVLHEYVNAAGITGGKSQFRV...
CD8-CIMPR-GFP11,
(SEQ ID NO: 526)
...DDSDEDLLHVGSSGSS(RDHMVLHEYVNAAGITGGSGG)₆RDHMV
LHEYVNAAGITGGK.

Plasmids CNX-S1-10(N) and CNX-S1-10(C) were obtained from Bernard Moss (NIH) and designated here GFP1-10-CNX and CNX-GFP1-10, respectively.

To construct pHPV16sheLL-GFP11-CPP for producing PsVs containing GFP11 inserted into the HPV16 L2 protein immediately upstream of the basic segment, DNA encoding the tandem GFP11 repeats was amplified from pGFP11-CD8-CIMPR by using the In-Fusion HD cloning Kit and cloned in-frame into L2 in p16sheLL. pHPV16sheLL-CPP-GFP11 with the GFP 11 repeats at the extreme C-terminus of L2 was constructed by first inserting Nsil and AvrlI restriction sites immediately upstream of the L2 stop codon in p16sheLL. The DNA segment encoding the GFP11 repeats was then inserted into this plasmid between these sites. Site-directed mutagenesis of pHPV16sheLL-GFP11-CPP and pHPV16sheLL-CPP-GFP11 was used to construct pHPV16sheLL-GFP11-3R and pHPV16sheLL-3R-GFP11, respectively, both of which contain three arginines in place of the wild-type CPP. The sequences of the relevant portions of the encoded L2-GFP 11 fusion proteins with wild-type CPP are as follows, with GFP 11 and linker sequences in bold and L2 CPP underlined.

L2-GFP11-CPP,
(SEQ ID NO: 527)
...FYLHPSYYML(RDHMVLHEYVNAAGITGGSGG)₆RDHMVLHEYVNAA
GITGGKFRKRRKRLPYFFSDVSLAA.
L2-CPP-GFP11,
(SEQ ID NO: 528)
...YYMLRKRRKRLPYFFSDVSLAADASGSSGSS(RDHMVLHEYVNAAGI
TGGSGG)₆RDHMVLHEYVNAAGITGGKFPRL.

Generation of stable cell lines. To generate stable cell lines expressing cytoplasmic GFP1-10NES, lentiviruses were produced by co-transfection of 293T cells in 100 mm dishes with 6 μg of pLentiCMV.GFP1-10NES, 4.5 μg of lentiviral packaging plasmid psPAX2 and 1.5 μg envelope plasmid pMD2.G. Forty-eight h later, the lentiviral supernatant was harvested, filtered and stored at −80° C. for later use. Cells stably expressing GFP1-10NES were constructed by infecting HaCaT cells with lentivirus for 48 h in medium containing 1 μg/mL puromycin. Single cells were then plated in a 96-well plate, and monoclonal cell lines were isolated and tested by infection of HPV16-CPP-GFP11 PsV at ˜2000 MOI and monitoring fluorescence.

Immunofluorescence microscopy of GFP1-10 expression. 5×10⁴ clonal HaCaT/GFP1-10NES stable cells grown on glass coverslips were fixed for 15 min at room temperature with 4% Formalde-Fresh, washed with PBS and incubated with anti-GFP mouse antibody (Santa Cruz, Dallas, TX, #SC-9996) and Alexa Fluor 488 donkey anti-mouse IgG (H&L) secondary antibody (Fisher, #A-21202). Images were acquired with a Leica SP5 inverted fluorescence microscope to identify cell lines expressing cytoplasmic GFP1-10NES.

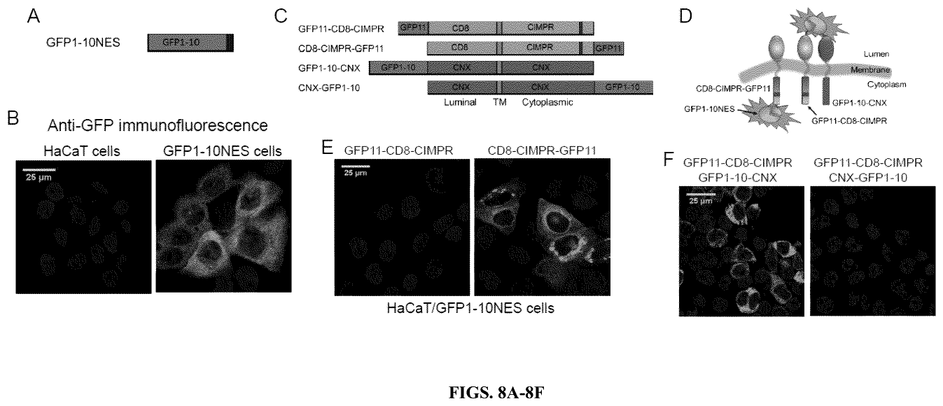

Validation of the split GFP assay. 5×10⁴ clonal HaCaT/GFP1-10NES cells in eight-chambered glass slides were transfected with 0.25 μg of pGFP11-CD8-CIMPR or pCD8-CIMPR-GFP11. Twenty-four hours later, live cells were examined by fluorescence microscopy. To demonstrate the functionality of GFP11 in GFP11-CD8-CIMPR, 5×10⁴ 293T cells in eight-chambered glass slides were co-transfected with a plasmid expressing GFP11-CD8-CIMPR and a plasmid expressing a calnexin transmembrane fusion protein containing luminal GPF1-10 or cytoplasmic GFP1-10 (GFP1-10-CNX and CNX-GFP1-10, respectively) (FIGS. 8C and 8D). Twenty-four h later, live cells were examined by fluorescence microscopy. As expected, fluorescence with GFP11-CD8-CIMPR was reconstituted only when the GFP1-10 segment was luminal (FIG. 8F).

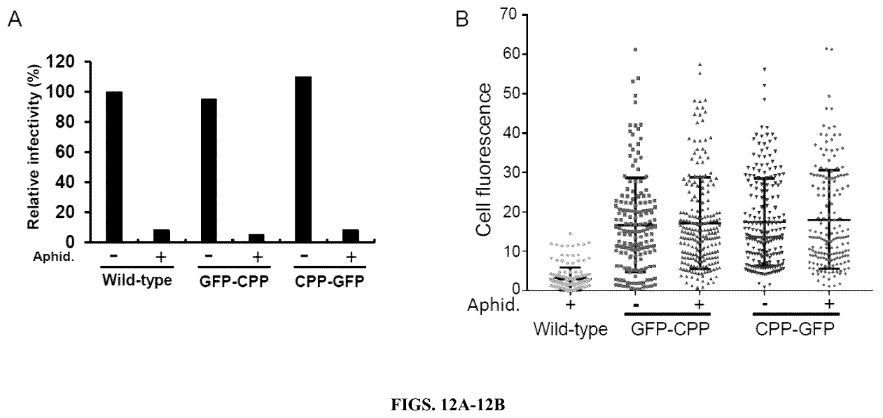

Use of split GFP assay to demonstrate cytoplasmic protrusion of L2. 3×10⁴ clonal HaCaT/GFP1-10NES cells seeded in eight-chambered glass slides were incubated for 1.5 or 3 h at MOI of ˜2000 with HPV16 PsV containing wild-type L2 or L2 containing inserted GFP11 and a wild-type or 3R mutant CPP. Live cells were analyzed by a Leica SP5 inverted fluorescence microscope and processed with ImageJ. In the experiment shown in FIG. 12B, cells were pretreated with aphidocolin, which was left in the medium for the duration of the experiment (Santa Cruz, #201535).

### Modulation of CPP Action by Flanking Sequences

Fusion proteins with various segments of the C-terminus of HPV16 or HPV5 L2 were added to HaCaT cells. The sequences used are as follows:

GFP- His6-GFP-linker CPP
(SEQ ID NO: 529)
-RKRRKR
GFP-21P-CPP His6-GFP-linker-
(SEQ ID NO: 530)
PQYTIIADAGDFYLHPSYYMLRKRRKR
GFP-HPV16 L2 His6-GFP-linker-
(SEQ ID NO: 531)
PQYTIIADAGDFYLHPSYYMLRKRRKRLPYFFSDVSLAA
GFP-HPV5 L2 His6-GFP-linker-
(SEQ ID NO: 532)
IPVVIIHPHDSTGDFYLHPSLHRRKRKRKYL

In some cases, various concentrations of fusion proteins were added to assess efficacy. In other cases, the pH of the culture medium was physiological pH 7 or adjusted to acidic pH 4 prior to peptide addition, or the cells were incubated with peptide at physiological temperature of 37° C. or at 30° C. Activity was assessed by confocal microscopy for intracellular fluorescence. In some experiments, cell surface fluorescence was extinguished by treatment with the membrane-impermeable fluorescence quencher, trypan blue.

### Peptide Inhibition Experiment

HeLa cells were left untreated or treated at 37° C. with 4 μg L2-C, a peptide containing the HPV16 L2 retromer binding site and CPP. The sequence of L2-C is as follows: B-SPQYTIIADAGDFYLHPSYYMLRKRRKR-Am, (SEQ ID NO: 533), where B represents N-terminal biotinylation and Am represents C-terminal amidation. Two hours later, the cells were infected for two hours at 37° C. with wild-type HPV16 pseudovirus. Cells were washed and medium was replenished with medium containing the peptide for 24 hours. Infectivity was tested 48 hours later by using flow cytometry to measure fluorescence of HcRed expressed from a reporter plasmid encapsidated in the pseudovirus particle, and normalized to infectivity of cells infected in the absence of peptide. Similar results were obtained in two independent experiments.

### Example 1: The C-Terminal Basic Sequence of L2 can be Replaced by a Cationic Cell-Penetrating Motif

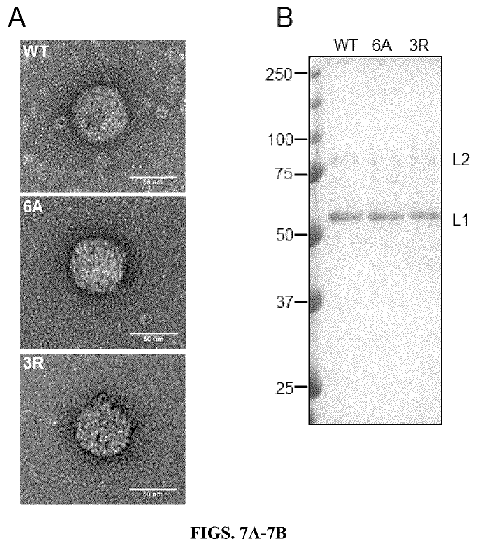

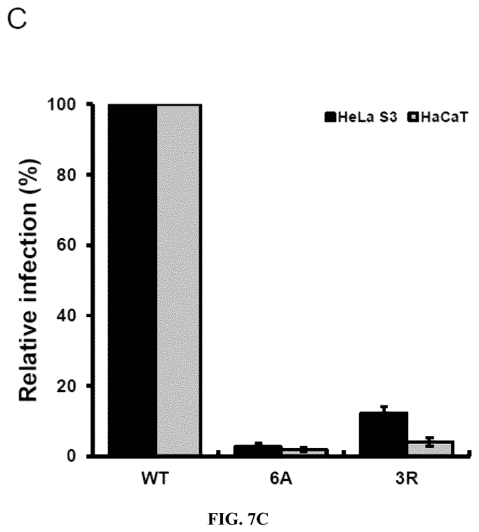

Direct binding of retromer to a carboxy-terminal segment of the HPV16 L2 protein is required for transport of the incoming virus from endosomes to the Golgi. To test whether the basic amino acids in the C-terminus of the L2 protein function as a CPP to transfer a segment of the L2 protein into the cytoplasm to allow a direct interaction with retromer, it was first tested whether mutations in this segment of L2 inhibited infectivity. For these experiments, pseudoviruses (PsVs) comprised of an HcRed reporter plasmid, wild-type HPV16 L1, and wild-type or mutant HPV16 L2 were used. PsV assembly was confirmed by electron microscopy, which showed no obvious morphologic differences between wild-type and mutant PsVs (FIG. 7A). Each PsV stock was normalized to the number of encapsidated reporter plasmids and analyzed by SDS-PAGE. Wild-type and mutant PsVs displayed comparable purity and contained similar levels of L1 and L2 (FIG. 7B).

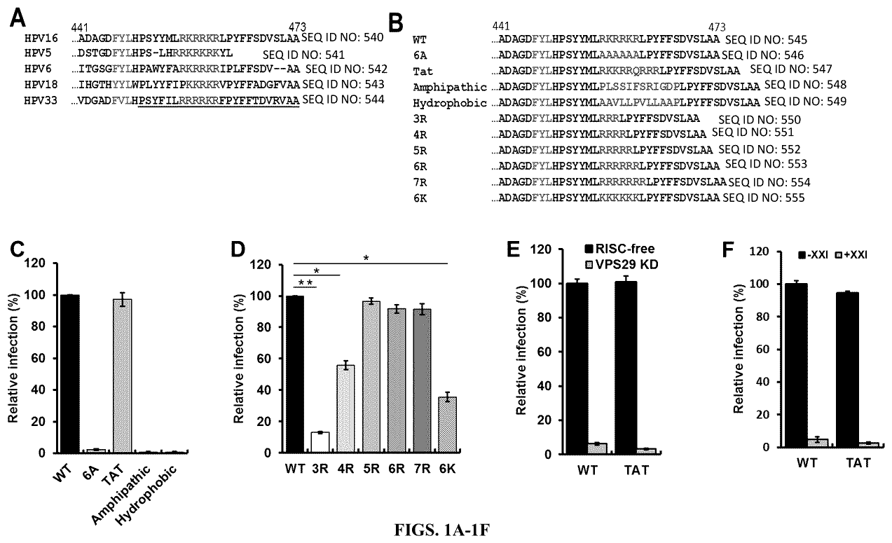

To determine if the L2 basic sequence is important for HPV16 infection, wild-type RKRRKR (SEQ ID NO: 2) was replaced with six alanines (6A mutant) (FIG. 1). HeLa or HaCaT cells were infected with wild-type HPV16 PsV at multiplicity of infection (MOI) of one or with 6A mutant PsV containing an equivalent number of encapsidated reporter plasmids, and infection efficiency was measured two days later by flow cytometry for HcRed fluorescence. As shown in FIG. 1C and FIG. 7C, the 6A mutation abolished infectivity, indicating that the basic sequence is essential for HPV16 infection. Next, the basic sequence of L2 was replaced with the CPP domain of the HIV-1 Tat protein to generate L2-Tat (FIG. 1). Strikingly, HPV16 PsV with the Tat sequence in place of the basic region infected cells as well as HPV16 PsV containing wild-type L2 (FIG. 1C). In contrast, an amphipathic or a hydrophobic CPP did not support infection. These results suggest that the basic region of L2 acts as a cationic CPP to deliver the C-terminus of L2 containing retromer binding sites through a membrane into the cytoplasm and that only the cationic class of CPP can support HPV infection.

RKRRKR (SEQ ID NO: 2) was then replaced with six consecutive lysines or with various numbers of arginines (FIG. 1i). A stretch of five or more arginines can act as efficient CPPs, whereas fewer arginines and polylysine were less effective. Infectivity of the mutant PsV with three arginines (3R) was minimal (˜10%) and increased with the number of arginine residues, reaching a plateau with five arginines (FIG. 1D and FIGS. 7A-7C). The mutant with six lysines was ˜40% as infectious as PsV containing five or more arginines. These results demonstrate that the basic region at the C-terminus of HPV16 L2 can be functionally replaced by arginine-rich sequences with known CPP activity, and that infectivity correlated with predicted CPP activity.

It was next tested if the HPV16 PsV containing L2-Tat utilized a similar entry pathway as wild-type HPV16 PsV. To determine if retromer was required for infection mediated by L2-Tat, cells transfected with siRNA that knocked down the Vps29 retromer subunit were infected.

Retromer knockdown dramatically inhibited infection by either wild-type or the L2-Tat PsV, indicating that the L2-Tat chimera requires retromer (FIG. 1E). γ-secretase is also required for proper trafficking by HPV16. To determine if γ-secretase was required for PsV containing L2-Tat, cells were treated with a chemical inhibitor of γ-secretase, compound XXI, and infectivity was measured. XXI caused near-complete inhibition of both wild-type and L2-Tat PsV infection, showing that the L2-Tat chimera also requires γ-secretase (FIG. 1F). Thus, the L2-Tat chimeric HPV16 PsV displays two of the key entry requirements as wild-type HPV16 PsV, namely dependence on retromer and γ-secretase. Taken together, these results provide strong genetic evidence that the basic segment of L2 acts as a CPP during HPV16 infection.

### Example 2: The Basic Sequence of L2 can Translocate Peptides and GFP Fusion Proteins into Cells

Experiments were conducted to determine whether the basic sequence of L2 has intrinsic cell penetrating activity. First, Alexa Fluor 488 was conjugated onto the N-terminus of a 28-residue L2 peptide that terminates with the wild-type basic sequence or the corresponding L2 peptide with the 6A or the 3R mutation (FIG. 2A, top). As assessed by confocal microscopy, the wild-type peptide entered cells, but the peptide containing 6A mutation did not (FIG. 2A, bottom). The 3R peptide displayed less cell-associated fluorescence signal than wild-type, suggesting that the 3R peptide is partially impaired for cell penetration.

To confirm CPP activity of this L2 segment, GFP fusion proteins were expressed and purified from bacteria consisting of GFP fused in-frame to a 28-residue segment from the C-terminus of L2 terminating with either the wild-type basic segment or a mutant segment (FIG. 2B). Microscopic examination of HeLa and HaCaT cells incubated with medium containing L2-GFP fusion proteins showed that the protein with the wild-type L2 segment generated a strong punctate fluorescence signal inside cells and at the cell periphery (FIG. 2C). The 3R mutation greatly impaired cellular uptake and the 6A mutation abolished it. To show that the wild-type fusion protein was internalized, cells incubated with fusion protein were treated with 0.04% trypan blue, a cell membrane-impermeable agent that quenches cell-surface GFP fluorescence but not intracellular fluorescence. As shown in FIG. 2C, in cells incubated with the wild-type fusion protein, the signal at the cell periphery was eliminated by trypan blue, but the punctate signal persisted, demonstrating intracellular uptake of the protein. In contrast, the signal from the 3R mutant was largely eliminated. To quantify cellular fluorescence at 0.5 to 5 h post-incubation, flow cytometry was performed following trypsinization to remove any GFP fusion protein adsorbed to the cell surface. This experiment showed that the wild-type fusion protein translocated into cells rapidly (with >50% and >90% of maximum fluorescence observed in HaCaT cells and HeLa cells by two hours, respectively), whereas there was no internalization of 6A and 3R fusion proteins (FIG. 2D). Thus, the wild-type and mutant basic segments of L2 displayed cell penetration activity that correlated with the infectivity of PsV containing these segments. These results provide strong evidence that the membrane-penetration property of the L2 CPP is required for HPV entry.

### Example 3: The L2 CPP is not Essential for Virus Binding and Internalization

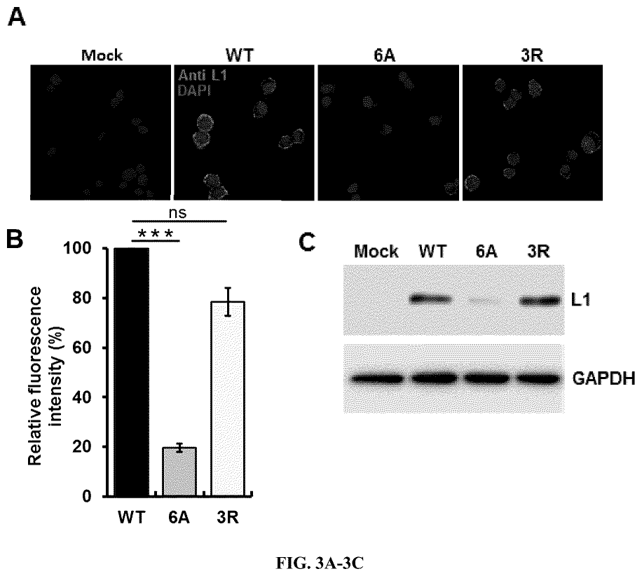

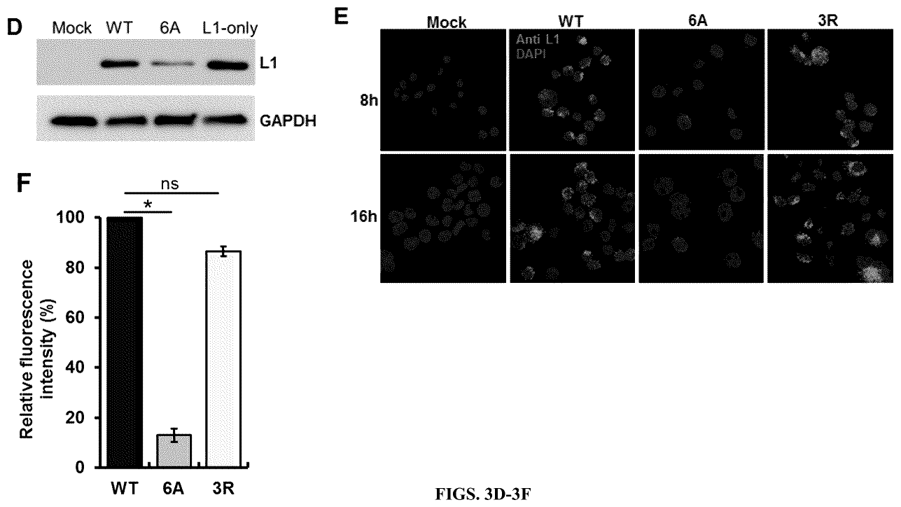

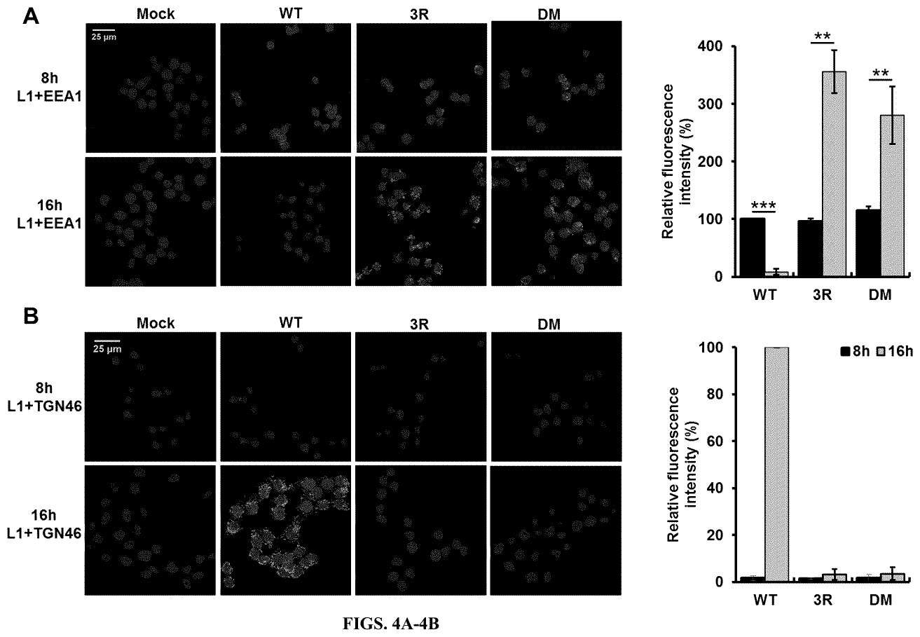

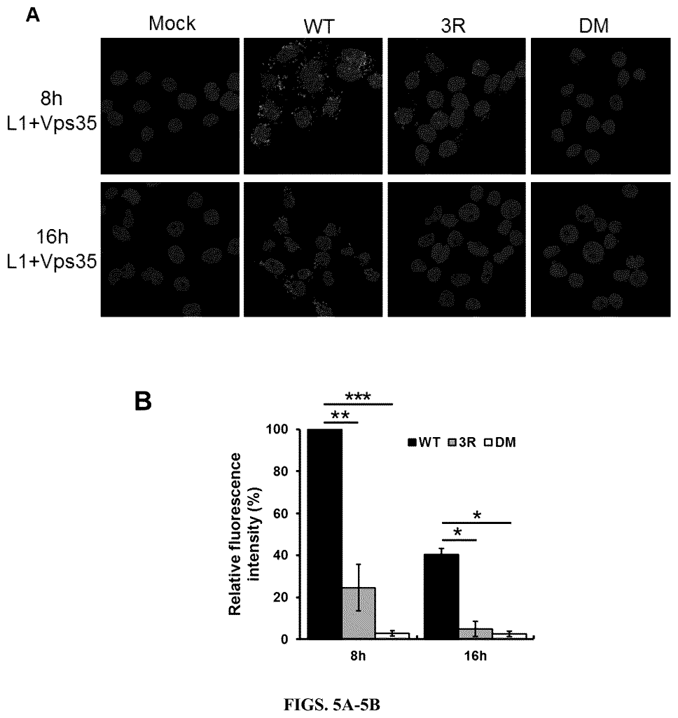

To determine the role of the L2 CPP in HPV infection, cell binding experiments were conducted. HeLa cells were incubated with either wild-type or mutant HPV16 PsV for two hours at 4° C., followed by washing to remove unbound viruses. Non-permeabilized cells were stained with an L1 antibody, and immunofluorescence was performed to detect viruses stably bound to cells. As shown in FIG. 3A, binding of wild-type PsV to cells was readily detectable, as was binding of the 3R mutant. Unexpectedly, the 6A mutation resulted in a dramatic reduction of cell binding. Similar results were obtained when cell binding was assessed by flow cytometry for L1 (FIG. 3B) or by western blotting (FIG. 3C) for L1 stably associated with cells incubated with HPV16 PsV at 4° C. Because the cell binding defect of the 6A mutant was in conflict with published studies showing efficient binding of capsids lacking L2, binding with PsV lacking L2 (L1-only PsV) was also tested. As shown in FIG. 3D, L1-only PsV bound cells to a similar level as complete PsV containing L1 plus wild-type L2, far better than PsV containing the 6A mutant. These results imply that the L2 CPP does not play a direct role in cell surface binding, but sequences in the C-terminus of L2 can modulate binding.

The role of the L2 CPP in virus internalization was examined next. After incubation of cells with HPV16 PsVs at 4° C., cells were shifted to 37° C. for six hours to allow internalization. Internalization was assessed by immunofluorescence and by flow cytometry. The 3R L2 mutant internalized into cells as well as wild-type, while the 6A mutant showed much less internalized L1, as expected because of its cell surface binding defect (FIGS. 3E and 3F). This result showed that the short basic segment of three arginines was able to support cell surface binding and virus internalization, even though it was not sufficient to restore infectivity. Thus, the L2 CPP is required for an intracellular event subsequent to cell binding and internalization.

### Example 4: The 3R Mutant Accumulates in Endosomes and Fails to Reach the Golgi

To examine the post-internalization defect of the 3R mutant, proximity ligation assay (PLA) was used to determine the localization of incoming wild-type and 3R mutant HPV16 PsV. PLA is an immune-based assay used to test if two proteins of interest are within 40 nm. PLA was performed with an anti-L1 antibody and an antibody that recognizes either EEA1, a marker of the early endosome, or the trans-Golgi network (TGN) marker, TGN46. The L2 double mutant (DM), which lacks retromer binding sites, was used as a control. As shown in FIG. 4A, these antibodies did not generate a PLA signal in mock-infected cells. At eight hours post-infection, punctate intracellular EEA1/L1 PLA signal was observed in cells infected with wild-type L2 or either mutant and showed similar fluorescence intensity, confirming that the 3R mutant, like DM, efficiently entered cells and reached the endosome. At 16 hours post-infection, the EEA1/L1PLA signal of cells infected with wild-type PsV was significantly diminished, reflecting the departure of the incoming virion from the endosome, whereas the signal for the DM in the endosome was markedly increased, reflecting endosomal accumulation due to the absence of retromer binding sites (FIG. 4A). Notably, the EEA1/L1 PLA signal of the 3R mutant at 16 h.p.i. was increased ˜3-fold compared to the eight-hour signal, similar to the increase seen with DM mutant. Thus, the 3R mutant and the DM accumulated in the endosome. As expected, little TGN46/L1 PLA signal was generated at eight hours after wild-type or mutant PsV infection (FIG. 4B). At 16 h.p.i., cells infected with wild-type displayed abundant TGN46 PLA signal, reflecting delivery of L1 to this distal site. In contrast, 3R and DM mutants showed a greatly reduced TGN46/L1 PLA signal, indicating that these mutants did not arrive at the TGN. These results indicate that 3R mutation leads to accumulation of the incoming virus particle in the endosome and prevents its arrival into the TGN, a phenotype similar to the mutant that cannot bind retromer. Together with the reduced infectivity of the 3R mutant and the impaired cell entry of 3R mutant fusion proteins, these data suggest that the trafficking defect displayed by 3R mutant is due to impaired endosomal membrane penetration and retromer association.

### Example 5: The 3R Mutant is Defective in Accessing Retromer During HPV Infection but Binds Retromer In Vitro

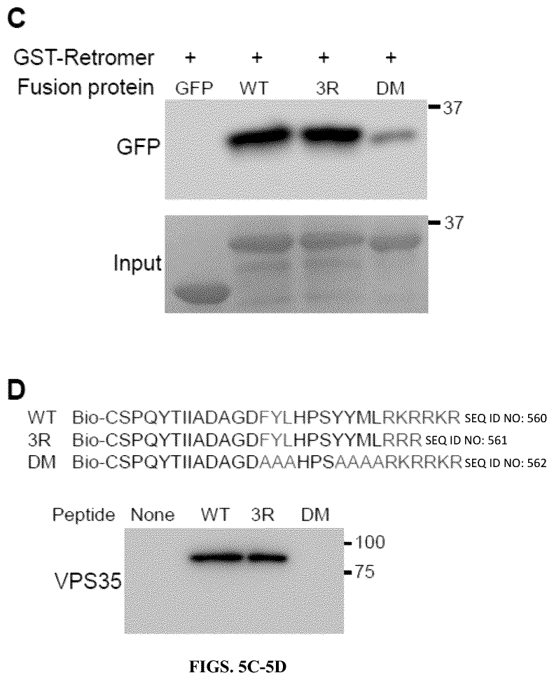

It was next tested whether the 3R mutation impaired association between the capsid and retromer in infected cells. HeLa cells were infected with either wild-type or mutant HPV16 PsV, and PLA was performed with an anti-L1 antibody and an antibody recognizing Vps35, a subunit of retromer. As shown in FIG. 5A, wild-type PsV generated abundant Vps35/L1 PLA signal at eight h.p.i., while the signal was diminished by 16 h.p.i., reflecting the exit of virus from endosomes and dissociation from retromer by this time. As expected, the PLA signal for DM mutant was very weak at both eight and 16 h.p.i., consistent with its lack of retromer binding sites. Interestingly, ˜75% reduction of PLA signal was observed for the 3R mutant at eight hours, and this signal decreased further by 16 hours (FIGS. 5A-5B), despite the accumulation of the mutant at the endosome at this later timepoint.

In certain embodiments, the 3R mutation can inhibit retromer association directly, by impinging on the retromer binding sites, or indirectly, by preventing the exposure of the binding sites in the cytoplasm. To determine if mutations in the L2 CPP directly inhibited binding to retromer, pull-down experiments were performed. GST-tagged retromer subunits were expressed in bacteria, assembled into the trimeric retromer complex, and bound to glutathione beads. The purified GFP-L2 fusion proteins containing a wild-type L2 segment, the 3R mutation, or the DM mutations in the retromer binding sites were incubated with the retromer beads at 4° C., pelleted, and subjected to western blotting with an anti-GFP antibody to detect the L2 fusion protein in the pellet. As expected, the wild-type protein bound retromer well and the DM fusion protein bound poorly (FIG. 5C). Notably, the 3R mutant L2 fusion protein also bound retromer well, demonstrating that this mutation did not inhibit the intrinsic ability of L2 to bind retromer. In addition, biotinylated, wild-type and mutant C-terminal L2 peptides were incubated with detergent lysates of uninfected HeLa cells and streptavidin pull-down experiments and western blotting were performed for Vps35. Similar to the results with purified fusion proteins, the 3R mutation did not affect retromer binding to the L2 peptide, whereas binding was abolished by the mutations in the retromer binding sites (FIG. 5D). Therefore, the 3R mutation does not directly interfere with retromer binding, and the 3R mutant displays impaired retromer association in infected cells because it cannot protrude through the endosomal membrane and access retromer in the cytoplasm.

### Example 6: Split GFP Assay Detects CPP-Mediated Cytoplasmic Exposure of the C-Terminus of L2

To directly demonstrate membrane passage of the L2 C-terminus during virus entry, a split GFP imaging method was adapted. A protein consisting of GFP beta strands 1 to 10 (GFP1-10) does not fluoresce, nor does the 16-residue eleventh beta strand of GFP (GFP11). However, when GFP11 is in the same cellular compartment as GFP1-10, holo-GFP is reconstituted, generating a fluorescent signal. This approach has been used to demonstrate cytoplasmic delivery of soluble fusion proteins linked to CPPs.

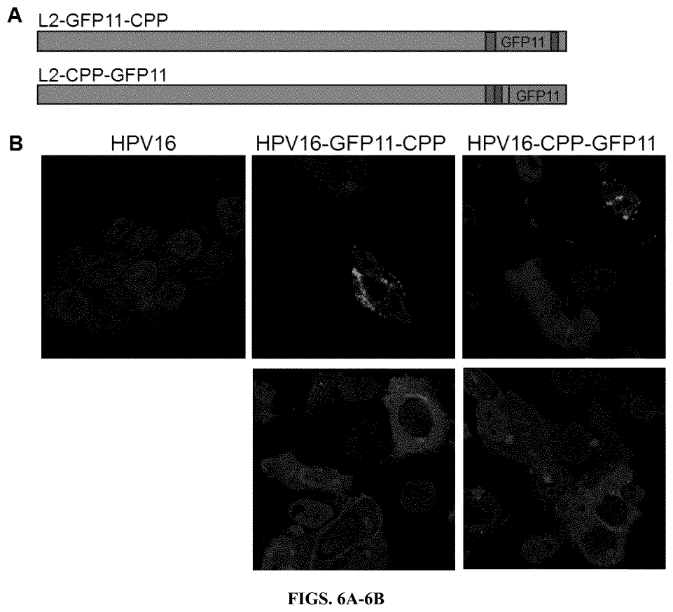

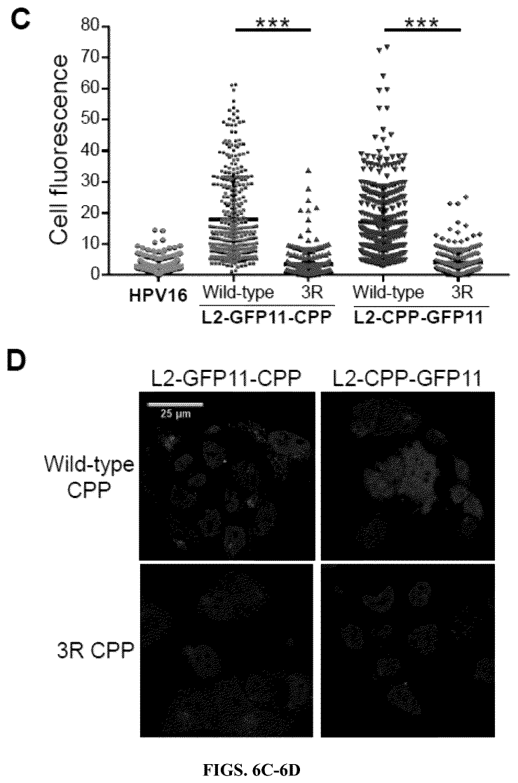

To use this assay to assess L2 exposure, a clonal HaCaT cell line expressing GFP1-10 linked at its C-terminus to a nuclear export signal (NES) was constructed (FIG. 8A). Immunofluorescence experiments with an anti-GFP antibody confirmed cytoplasmic expression of GFP1-10NES (FIG. 8B). As expected, cells expressing this construct without GFP11 displayed minimal fluorescence (see FIG. 8E and FIG. 6B). Control experiments were then conducted to establish that GFP fluorescence in HaCaT/GFP1-10NES cells indicated the presence of GFP11 in the cytoplasm. CD8-cation-independent mannose phosphate receptor (CD8-CIMPR) fusion proteins consisting of the extracellular domain of CD8 fused to the transmembrane and cytoplasmic domains of CIMPR were used. Seven tandem copies of GFP11 were inserted at either the N-terminus or the C-terminus of CD8-CIMPR to generate GFP11-CD8-CIMPR and CD8-CIMPR-GFP11, respectively (FIG. 8C). When expressed, these proteins adopt a type 1 transmembrane orientation with the GFP 11 segment located in the extracellular/luminal space for GFP11-CD8-CIMPR and in the cytoplasm for CD8-CIMPR-GFP 11 (FIG. 8D). Neither construct fluoresced on its own when transfected into unmodified HaCaT cells. HaCaT/GFP1-10NES cells were then transfected with a plasmid expressing GFP11-CD8-CIMPR or CD8-CIMPR-GFP11 and fluorescence was assessed. As shown in FIG. 8E, bright cytoplasmic fluorescence was observed after expression of CD8-CIMPR-GFP11 (with cytoplasmic GFP11) but not after expression of GFP11-CD8-CIMPR. Additional control experiments showed that luminal GFP1-10 reconstituted fluorescence in cells expressing GFP11-CD8-CIMPR (see FIGS. 8C, 8D, and 8F). Taken together, these results validate the split GFP reporter system by showing that fluorescence is reconstituted in HaCaT/GFP1-10NES cells only when the GFP11 segment is in the cytoplasm and shows that GFP1-10NES does not access the luminal space.

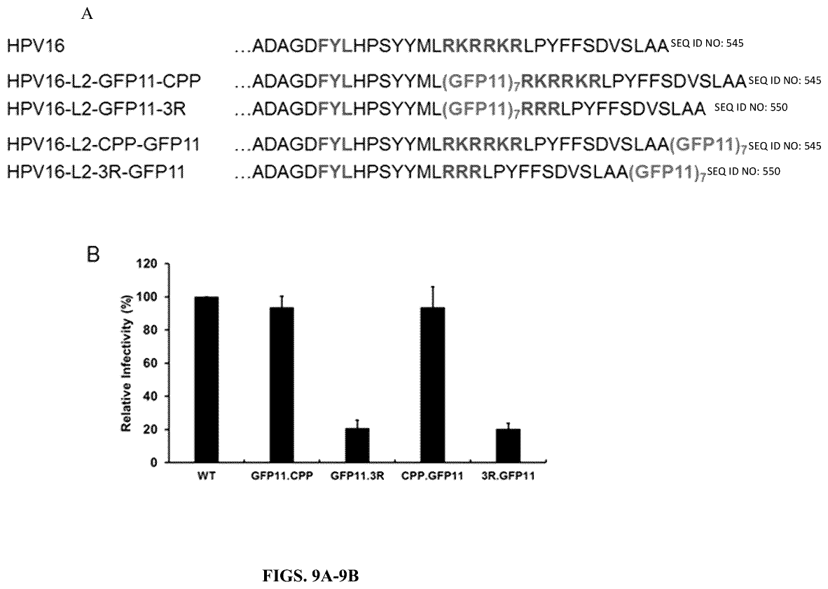

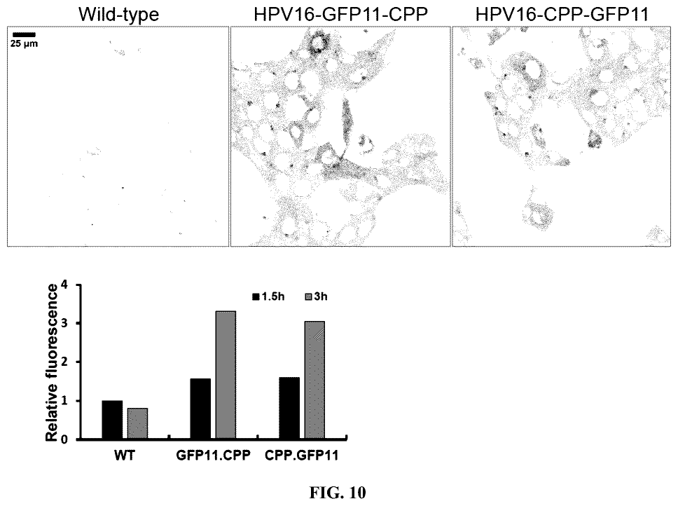

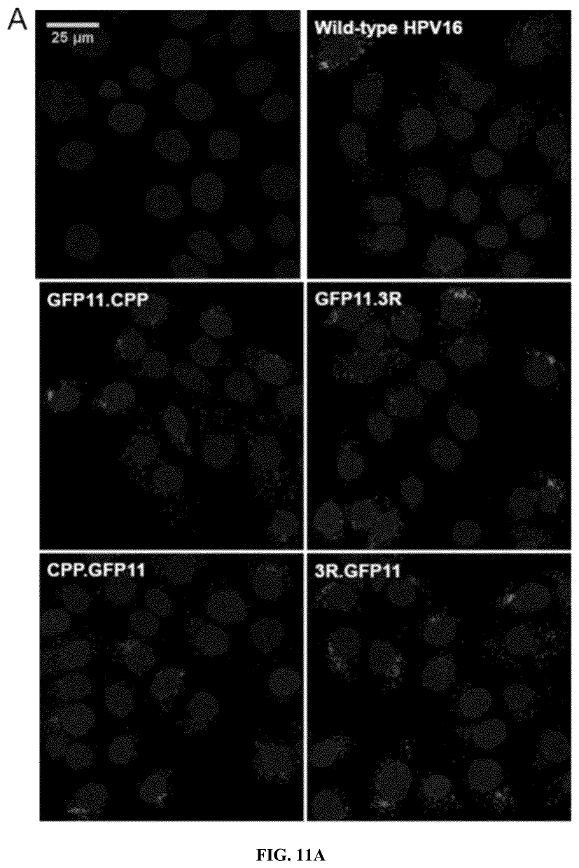

To assess cytoplasmic exposure of L2 during infection, seven tandem copies of GFP11 were inserted at two different positions in the C-terminus of the L2 protein: between the CPP and the retromer binding sites (L2-GFP11-CPP) or at the extreme C-terminus of L2 (L2-CPP-GFP11) (FIGS. 6A and 9A). Insertion of GFP11 into L2 did not impair HPV16 PsV infectivity (FIG. 9B). HaCaT/GFP1-10NES cells were then infected at high MOI with HPV16 PsV with or without GFP11-tagged L2 protein and the cells were examined by confocal microscopy. As shown in FIGS. 6B and 10, fluorescence was not detectable in HaCaT/GFP1-10NES cells infected with wild-type HPV16PsV lacking the GFP11 insert, confirming that the GFP1-10NES protein did not fluoresce on its own. Similarly, infection of unmodified HaCaT cells with HPV16 PsV containing GFP11-tagged L2 did not generate a fluorescent signal. Strikingly, however, infection of HaCaT/GFP1-10NES cells with the PsV containing GFP11 inserted at either C-terminal position in L2 resulted in reconstituted cytoplasmic fluorescence in ˜60-90% of cells (FIGS. 6B, 6C, and 10). Reconstituted fluorescence showed either a punctate distribution or a more uniform distribution throughout the cytoplasm (or both) and was evident at a low level as early as 1.5 h.p.i., which increased by 3 h.p.i (FIGS. 6B and 10). Notably, reconstituted fluorescence did not display peripheral, cell-surface localization at any timepoint examined. This result demonstrates that early during HPV infection, the C-terminus of at least a fraction of L2 molecules was exposed in the cytoplasm.

To test if the L2 CPP was required for membrane protrusion, the wild-type CPP in L2-GFP11-CPP and L2-CPP-GFP11 was replaced with three arginines (FIG. 9A). The 3R mutation inhibited infection but did not inhibit virus internalization (FIGS. 9B and 11A). In HaCaT/GFP1-10NES cells infected with these mutant PsVs, there was significantly less reconstituted fluorescence compared to cells infected with PsV with wild-type CPP (FIGS. 6C and 6D). This result provides direct evidence that the L2 CPP is required for cytoplasmic exposure of the L2 C-terminus during infection.

### Example 7

To assess the effect of sequences flanking the core basic CPP, fusion proteins were constructed and purified with varying portions of the C-terminal segment of HPV16 and HPV5 L2. HPV16 typically infects genital and oral epithelia, at body core temperature of 37° C., whereas HPV5 is a virus that infects skin, which is several degrees cooler. The ability of these L2 segments to allow the fusion proteins to bind to cells and be delivered intracellularly was tested under a variety of conditions. In aggregate, these experiments show 1) that the 21 amino acids upstream of the HPV16 L2 CPP stimulates the ability of fusion proteins to enter cells (FIG. 13); 2) that the 12 amino acids downstream of the HPV16 L2 CPP allows the fusion protein to preferentially enter cells at low pH and apparently inhibits uptake at neutral pH (FIG. 14); and 3) sequences encompassing the HPV5 L2 CPP and flanking amino acids allow preferential cell binding at 30° C. (FIG. 15). Taken together, these results indicate that sequences flanking the CPP can modulate its ability to bind and enter cells, and that the behavior of fusion proteins containing HPV L2 segments may mimic the activity of the virus in vivo.

### Example 8

A major long-term goal is to use CPPs to deliver bioactive peptides into cells as therapeutics. As an example of using the HPV L2 CPP to deliver a novel peptide-based inhibitor that blocks HPV infection. Intracellular delivery of peptides containing the retromer binding site (RBS) of HPV16 compete for retromer binding with the RBS on the L2 protein of the incoming virus particle protruding through the endosome membrane, and thus cause HPV to accumulate in the endosome. Cells were treated with peptide L2-C, comprised of a C-terminal segment of HPV16 L2 including the RBS and adjacent CPP. The CPP delivers the peptide across the plasma membrane into the cytoplasm where the RBS on the peptide will bind retromer and compete with the intact L2 protein on the incoming virion, thereby inhibiting infection. FIG. 17 shows that a wild-type peptide terminating at the end of the CPP causes a dramatic inhibition of infection by HPV16 PsV, as measured by expression of a reporter gene packaged into the virus particle. Therefore, the CPP delivers the peptide into the cytoplasm where it competes with the incoming virus particle for binding to the essential cellular trafficking factor retromer, thereby inhibiting infection.

### Example 9

In order to traffic to the nucleus, papillomaviruses rely on cellular retrograde transport, but it was not clear how the incoming virion in the endosomal lumen enters the retrograde pathway. Here, these data show that a short sequence of basic amino acids near the C-terminus of the L2 protein acts as a CPP to transfer a segment of the L2 protein into the cytoplasm where adjacent sequences can bind retromer for transport to the TGN. First, it was shown that the basic region of L2 is required for efficient infection of epithelial cells and can be replaced with the cationic CPP from HIV-1 Tat. Like wild-type HPV16 PsV, PsV containing the Tat CPP required retromer and γ-secretase for infection. Five or more consecutive arginine residues restored full infectivity, whereas fewer arginines and six lysines were less effective, consistent with the known cell-penetrating activities of these sequences. Peptide and protein transduction assays were used to demonstrate that the basic segment of L2 did in fact display CPP activity. Importantly, a truncated sequence of three arginines was defective for CPP activity and failed to support infection. The preferential uptake of the 3R peptide compared to the 3R fusion protein may reflect the much higher concentration of molecules used in the peptide experiments. Taken together, these experiments demonstrated that L2 CPP activity was required for HPV infection.

The presence of a C-terminal basic region in all papillomavirus L2 proteins implies that the essential role of the L2 CPP has been maintained since the papillomaviruses first emerged more than 250 million years ago. The amino acid sequence of the basic segment is variable, consistent with the relatively relaxed sequence requirements for CPPs. The 353 sequenced L2 proteins in the papillomavirus PaVe sequence database (pave dot niaid dot nih dot gov/#home) contain 164 different C-terminal basic sequences, including a 10-residue poly-arginine stretch in three canine viruses (Table 1). RKRRKR (SEQ ID NO: 2) present in HPV16 L2 is one of the most common, being found in 16 diverse human and animal papillomaviruses, and many more CPPs are likely to exist in the papillomavirus virome because most of these different basic sequences have been identified in only a single virus type (Table 1). In addition, sequences flanking the core basic amino acids may influence membrane penetration activity. The multitude of papillomavirus types thus represents the results of a mutational analysis carried out over evolutionary time, revealing hundreds of different, presumably non-toxic sequences that can have cargo-carrying activity.

HPV16 PsV containing a mutant CPP consisting of three arginines was internalized, showing the L2 CPP was not required for endocytosis, but the mutant was defective for retromer engagement, exit of the virus from the endosome, and trafficking to the TGN. The same phenotype is caused by retromer binding site mutations. However, the CPP mutation did not directly impair the ability of L2 to bind retromer, implying that during infection the retromer binding sites were not in the same cellular compartment as retromer.

To directly assay the cytoplasmic exposure of L2, a split GFP assay was developed, in which fluorescence is reconstituted when a segment of GFP at the C-terminus of L2 encounters GFP1-10 in the cytoplasm. Cytoplasmic exposure of L2 was detectable early during infection and was impaired by replacing the CPP with three arginines. These findings show that the L2 CPP mediates passage of the C-terminus of the L2 protein into the cytoplasm so that it can engage retromer and enter the retrograde trafficking pathway. In some cells, reconstituted fluorescence is fairly uniform throughout the cytoplasm (FIGS. 6B and 10), suggesting that the C-terminal segment of L2 can be cleaved from the virion after protrusion through the membrane or that retrograde transport vesicles containing virions rapidly distribute through the cytoplasm.

The rapid generation of reconstituted GFP fluorescent signal is consistent with the fact that the L2 protein binds to the cytoplasmic protein SNX17 as early as two h.p.i. In addition, by one and a half to three h.p.i., intracellular L1 is detectable by immunofluorescence with the 33L1-7 conformation-specific antibody (FIG. 11B) and by western blotting, providing additional evidence that HPV internalization can occur quite rapidly. To determine if L2 protrusion also required cell cycle progression, aphidocolin, which causes S-phase arrest and blocks HPV infection by inhibiting translocation of the incoming virus into the nucleus late during entry, was used. As shown in FIG. 12, aphidocolin inhibited infection but did not decrease the reconstituted GFP signal, showing that cell cycle progression and nuclear membrane breakdown are not required for L2 exposure. Thus, GFP reconstitution indicates the protrusion of L2 molecules into the cytoplasm to engage the retrograde trafficking machinery in the first place. The split GFP assay should be useful in investigating the action of additional chemicals and mutations that inhibit HPV trafficking.

The physiological role of most CPPs is not known. Naturally-occurring CPPs are usually studied as small peptide fragments removed from their protein of origin, and in many cases cell-penetrating activity may be the fortuitous consequence of basic sequences with no natural role in membrane penetration. These results show that CPP-driven membrane penetration by L2 plays an important role in HPV infection and that the L2 CPP of endocytosed virus protrudes through the endosomal membrane into the cytoplasm. The bulk of L2 then passes through the membrane, possibly assisted by endosomal acidification, until it is arrested by its N-terminal transmembrane domain in a type 1 transmembrane orientation with its N-terminus in the endosomal lumen and most of the protein exposed in the cytoplasm. The exposed C-terminus then binds to essential entry factors, including retromer, which sorts the virus into retrograde transport vesicles that later fuse with more distal retrograde compartments. The effect of the L2 CPP on the membrane is relatively subtle and localized, in contrast to the more drastic membrane disruption events caused by other non-enveloped viruses, which are deposited into the cytoplasm. This allows the residual HPV virion, including viral DNA, to be retained in transport vesicles and presumably contributes to the relatively low immunogenicity of these viruses during cell entry by sequestering them from cytoplasmic immune sensors. The L2 protein may protrude into the cytoplasm in a sequential fashion, with the C-terminus being exposed prior to the middle of the protein. Thus, cellular proteins may bind L2 sequentially, first retromer to the C-terminus of L2 and later proteins such as SNX17 to the middle portion of L2. Such ordered binding may be important for the assembly of the protein complexes necessary for proper trafficking. It also is important to note that each capsid contains up to 72 L2 proteins. The presence of multiple L2 molecules in each virion ensures a high local concentration of CPPs upon infection even at low MOI, which may be important for membrane penetration activity. However, it is possible that not all L2 molecules insert into membranes or bind retromer or other cytoplasmic factors.

The experiments reported here show that the L2 protein is an inducible transmembrane protein. In the intact capsid, which lacks membranes, L2 cannot have any transmembrane character. However, once the L2 CPP inserts into the membrane and protrudes into the cytoplasm, the L2 protein adopts a transmembrane existence. Thus, CPPs can not only transport molecules into cells or between compartments, but can also transform a soluble protein into a transmembrane one. Cellular and other viral proteins may also transition from a soluble to a transmembrane state, with important consequences for their biological activities. These results suggest that the primary role of CPPs in biology may not be to transfer proteins into cells in a paracrine fashion but rather to act intracellularly to mediate the transfer of proteins or protein segments between cellular compartments and to convert soluble proteins into transmembrane proteins.

The Materials and Methods employed in Example 10 are here described.

### Cells, Plasmids, and Virus

HeLa-S3 cells were obtained from the American Type Culture Collection (ATCC). HaCaT cells purchased from AddexBio Technologies are spontaneously transformed keratinocytes from histologically normal skin. 293TT cells, generated by introducing SV40 Large T antigen cDNA into 293T cells to increase SV40 Large T antigen expression. All cells were cultured in DMEM with HEPES and L-glutamine, supplemented with 10% fetal bovine serum, and 100 units/mL penicillin-streptomycin at 37° C. in 5% CO2.

The plasmids designated p16sheLL, p18sheLL, and p5sheLL, expressing both L1 and L2 for HPV16, HPV18, and HPV5 pseudovirus production, respectively. pCAG-HcRed reporter plasmid was purchased from Addgene.

HPV PsV was produced by co-transfecting 293TT cells with pCAG-HcRed and a psheLL expression plasmid with or without mutations at the C terminus of L2. The packaged PsV was purified by density gradient centrifugation in Opti Prep. Quality of PsV preparations was confirmed by SDS-PAGE, followed by Coomassie brilliant blue staining for L1 and L2. SV40 was prepared in CV1 cells as described.

The infectious multiplicity-of-infection (MOI) of HPV PsV was assessed by flow cytometry for reporter gene expression after infection of HeLa-S3 cells with wild-type HPV16 PsV. The number of packaged reporter plasmids required to achieve the MOI for wild-type PsV was quantified by qPCR, and an equivalent number of genomes in mutant PsV was used to infect cells. For quantifying encapsidated HcRed genomes, purified PsV was treated with DNase I to remove free DNA associated with capsids. Reporter DNA genome was isolated using a DNA purification kit, and the copy number of encapsidated reporter plasmid was determined by qPCR using primers for the HcRed gene in comparison to a standard curve.

### Preparation of Peptides

Peptides were purchased from ABclonal Technology at >95% purity. Peptide (sequences shown in FIG. 1A) was resuspended in 30% acetic acid and dimethyl-formamide (˜30 μl each, 2.7% stock concentration, 0.02% working concentration), and then dissolved in sterile deionized water with 0.01% sodium azide. Peptide stocks (˜5 mg/mL) were aliquoted and stored at −20° C.

### Infectivity

To assess the effect of peptides on viral infection, 5×104 HeLa-S3 in 24-well plates were pretreated with peptides for one hour, followed by infection with wild-type HPV PsV at 37° C. at MOI of ˜1 or with mutant PsV containing an equivalent number of encapsidated reporter plasmids. Peptides were left in the medium for the duration of the experiment unless otherwise indicated. As a control, cells were incubated with the solution used to dissolve peptide. Cells were assessed by flow cytometry on a Stratedigm-13 flow cytometer to determine reporter protein expression at 48 h.p.i. unless otherwise indicated. In some experiments, peptides were added at various times after infection and were left in the medium for the duration of the experiment.

To measure the inhibition of infection by authentic HPV16, HeLa cells were infected with 5 μL raft-derived HPV16 (obtained from Craig Meyers, Hershey Medical Center) or with HPV16 PsV in the presence and absence of 14 μM P16/16, and total RNA was isolated 48 h.p.i. by using the RNeasy kit following the manufacturer's instructions. RNA was reverse-transcribed into cDNA by iScript cDNA Synthesis kit. The cDNA was quantified in triplicate by using SYBR Green Supermix real-time PCR detection system and primers for HPV16 E7 or HcRed. Actin mRNA were used for normalization.

HcRed forward
(SEQ ID NO: 534)
GCACCCAGAGCATGAGAAT
HcRed reverse
(SEQ ID NO: 535)
TCGTAGGTGGTGGTTCTCT
HPV16 E7 forward
(SEQ ID NO: 536)
AATGTTTCAGGACCCACAGG
HPV16 E7 reverse
(SEQ ID NO: 537)
CTCACGTCGCAGTAACTGTTG
Actin forward
(SEQ ID NO: 538)
CTGCTGTCACCTTCACCGTTCC
Actin reverse
(SEQ ID NO: 539)
AGTACTCCGTGTGGATCGGC

For studies of SV40 infection, 7.5×105 HeLa-S3 in 6-well plates were incubated with peptides prior to infection. Cells were infected with a crude preparation of SV40 at MOI of ˜1 and fixed with 4% paraformaldehyde at 48 h.p.i. Samples were stained with FITC-conjugated anti-large T antigen antibody (Pab101) (Santa Cruz, pSC147 FITC), followed by flow cytometry to determine the mean fluorescent intensity of T antigen staining.

### Immunofluorescence Microscopy and Proximity Ligation Assay

For the internalization experiments, 5×104 HeLa-S3 cells were grown on glass coverslips for 16 hours. After one-hour incubation with or without peptides, PsV at MOI of 50 were added and incubated at 4° C. for 2 hours, washed with PBS three times to remove loosely bound PsV, and shifted to 37° C. to initiate infection. As a control, cells were incubated with the solution used to dissolve peptides alone. At the indicated times post-infection, samples were fixed, permeabilized and stained with anti-L1 and AlexaFluor 488 conjugated secondary antibody. Cells were analyzed by a Leica SP5 confocal microscope.

For the proximity ligation assay, after an hour incubation with peptides, HeLa-S3 cells were infected with wild-type PsV at MOI of 200 or mutant PsV containing the same number of reporter plasmid genomes. Infected cells were fixed at 8 or 16 h.p.i., permeabilized, and incubated with 1:100 dilutions of anti-L1 antibody and an antibody recognizing EEA1, TGN46, or VPS35. PLA was performed with Duolink reagents from Olink Biosciences according to the manufacturer's directions. Briefly, after staining with primary antibody, cells were incubated with a pair of suitable PLA antibody probes at 1:5 in a humidified chamber and processed for ligation and amplification with fluorescent substrate at 37° C. The nuclei were stained by using Abcam fluorescence mounting medium with DAPI and images were acquired by a Leica SP5 inverted fluorescence microscope. Approximately 200 nuclei in each sample were imaged. The images were processed by Fiji and quantitatively analyzed by BlobFinder software to measure total fluorescence intensity in each sample. The average fluorescence intensity per cell in each sample was normalized to the control sample as indicated in each experiment. All the experiments were done independently three times with similar results.

### DMT1-II Localization

3×104 HeLa-S3 cells were transfected with 0.5 μg of a plasmid expressing a GFP DMT1-II fusion protein. Six hours later, medium was removed. 14 μM P16/16 or PDM/16 was added or cells were left untreated overnight. Medium was replaced with fresh medium containing same concentration of peptide, and cells were fixed and stained overnight with 1:200 dilution of anti-TGN46 antibody and then incubated with secondary antibody that recognized anti-TGN46. Cells were mounted with Abcam mounting medium with DAPI, imaged on a Leica SP5 confocal microscope, and processed with Fiji.

### Split GFP Assay

To determine if the cytoplasmic protrusion of L2 of PsV is inhibited by peptides, the split GFP assay was performed. 3×104 clonal HaCaT/GFP1-10NES expressing GFP1-10NES in the cytoplasm were seeded in eight-chambered glass slides and incubated with peptides for an hour prior to infection at MOI of ˜2000 with HPV16 PsV containing wild-type L2 or L2 containing inserted GFP11. Live cells were stained with Hoechst 33342, examined by a Leica SP5 confocal microscope, and processed with Fiji.

The inhibition efficiency of peptides at high MOI was tested by incubating cells with peptides at 30 μM peptide for two hours prior to infection with PsV at MOI of 2,000. At three h.p.i., cells were washed with PBS three times to remove unbound viruses and then incubated at 37° C. in the presence of 30 μM peptide. Forty-eight hours later, cells were assessed by flow cytometry to determine reporter protein expression.

### Example 10

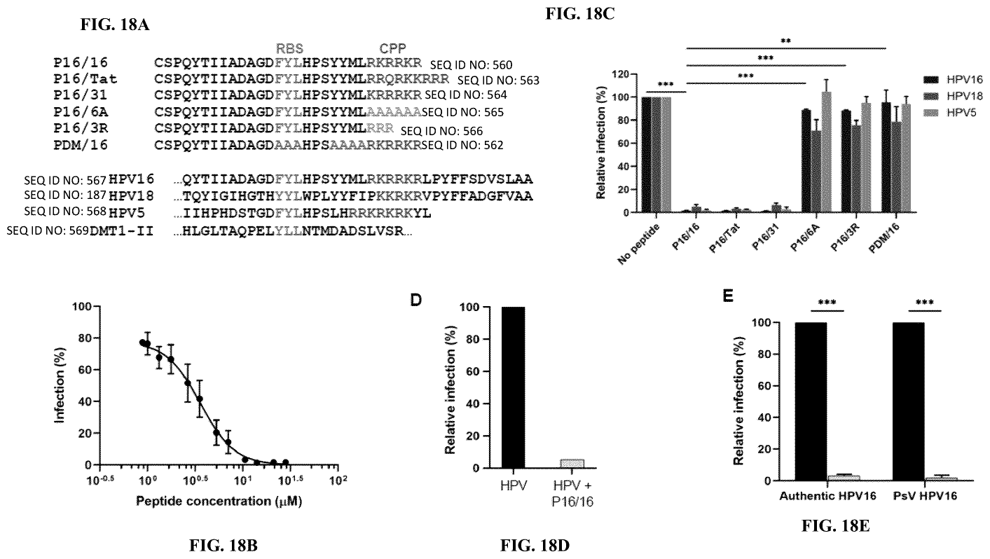

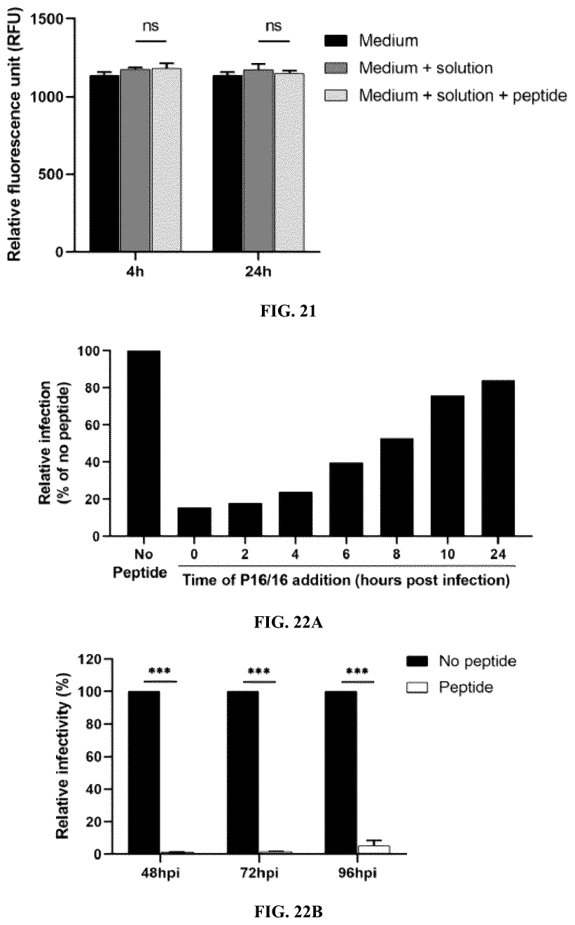

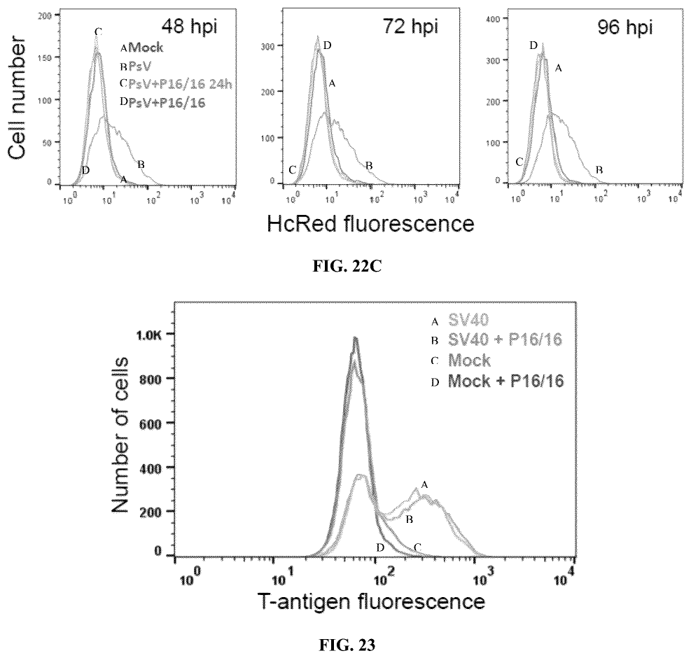

The utility of potential peptide therapeutics with an intracellular site of action is limited by inefficient delivery of the peptide into cells. The L2 CPP can deliver peptides and fusion proteins into cells if added to the culture medium. We reasoned that a segment of L2 containing the CPP would deliver the adjacent RBS into the cytoplasm, sequester retromer from incoming HPV, and block HPV infection. We synthesized a 29-residue peptide, designated P16/16, that contains the RBS and CPP (RKRRKR) (SEQ ID NO: 2), where R represents arginine and K represents lysine) from HPV16 L2 (FIG. 18A). This peptide is competent for retromer binding in vitro. HeLa cells were pre-incubated for one hour in medium containing various concentrations of peptide and then infected at a multiplicity of infection (MOI) of 1 with HPV16 pseudovirus (PsV), which consists of a complete L1 and L2 capsid containing a plasmid that expresses the fluorescent protein, HcRed. As shown in FIG. 18B, the L2 peptide caused a dose-dependent reduction in infection, as assessed by flow cytometry for HcRed fluorescence 48 hours post-infection (h.p.i.). Under these conditions, P16/16 displayed an IC50 of 3.6 μM and caused no apparent toxicity based on morphology and cell viability assay (FIG. 21). For effective inhibition, the peptide needed to be added during the first few hours of infection (FIG. 22A), and inhibition persisted until at least 96 h.p.i. (FIG. 22B). If P16/16 was removed from the medium after 24 hours, infection was not restored over the next three days (FIG. 22C). P16/16 also potently inhibited infection by HPV18 and HPV5 PsV (FIG. 18C). HPV18 is a high-risk oncogenic HPV type that infects genital mucosa like HPV16, and HPV5 is a divergent HPV type associated with skin cancer. All known HPV L2 proteins contain a recognizable RBS, suggesting this strategy will inhibit all HPV types.

Peptides containing the CPP from HIV Tat (P16/Tat) or HPV31 (P16/31) in place of the HPV16 CPP also markedly inhibited infection of the three HPV PsV types tested (FIGS. 18A and 18C). In addition, P16/16 inhibited infection by HPV16 PsV in HaCaT skin keratinocytes (FIG. 18D). P16/16 also inhibited authentic HPV16 produced in organotypic raft cultures, as assessed by quantitative reverse transcriptase PCR for viral mRNA (FIG. 18E), but did not inhibit SV40, an unrelated non-enveloped small DNA tumor virus that does not require retromer (FIG. 23). Importantly, mutations of the RBS (6A and 3R) or the CPP (DM) in P16/16, which block retromer binding and membrane penetration, respectively, eliminated the inhibitory effect (FIGS. 18A and 18C).

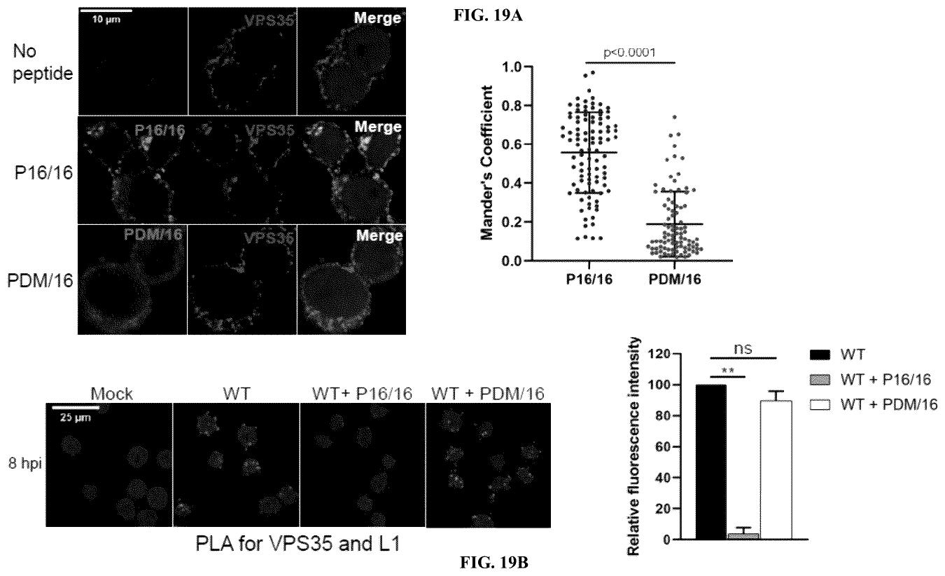

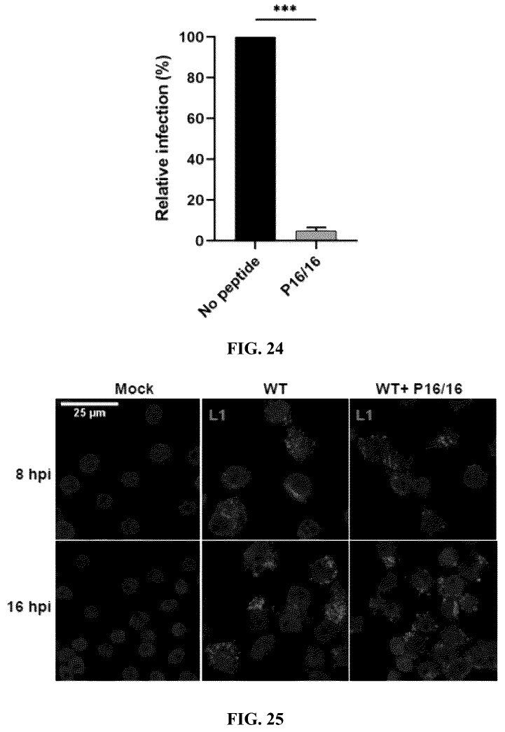

We used biotinylated peptides to confirm their transfer into cells and determine their intracellular location. Biotinylation did not affect anti-HPV activity (FIG. 24). Uninfected HeLa cells were incubated with biotinylated P16/16 or PDM/16, a mutant peptide lacking the RBS (designated bP16/16 and bPDM/16, respectively). Three hours later, cells were stained with streptavidin and an antibody that recognizes the retromer subunit, VPS35. bP16/16 was localized to large cytoplasmic puncta superimposed on a more diffuse cytoplasmic distribution (FIG. 19A). Strikingly, the mutant bPDM/16 showed only the diffuse distribution. These results show that the peptides rapidly enter cells and suggest that retromer binding is required for the punctate signal. VPS35 also displayed a punctate distribution in the presence or absence of peptide. Importantly, there was significant colocalization of wild-type bP16/16 and VPS35, which was greatly reduced with PDM/16, showing that the wild-type peptide but not the mutant stably associated with retromer in intact cells. To test whether the L2 peptide inhibited the ability of retromer to bind incoming HPV16 PsV in infected cells, we conducted a proximity ligation assay (PLA) for HPV16 L1 and the retromer subunit VPS35. PLA generates a fluorescent signal when two antigens are within 40 nm. FIG. 19B shows that eight h.p.i., PLA detected interaction of incoming virus with retromer in intact, infected cells. Strikingly, the wild-type P16/16 peptide inhibited the interaction of retromer with HPV, whereas the mutant peptide lacking the RBS did not. Together, these data show that the peptide containing the RBS associates with retromer and inhibits binding of retromer to the incoming virus.

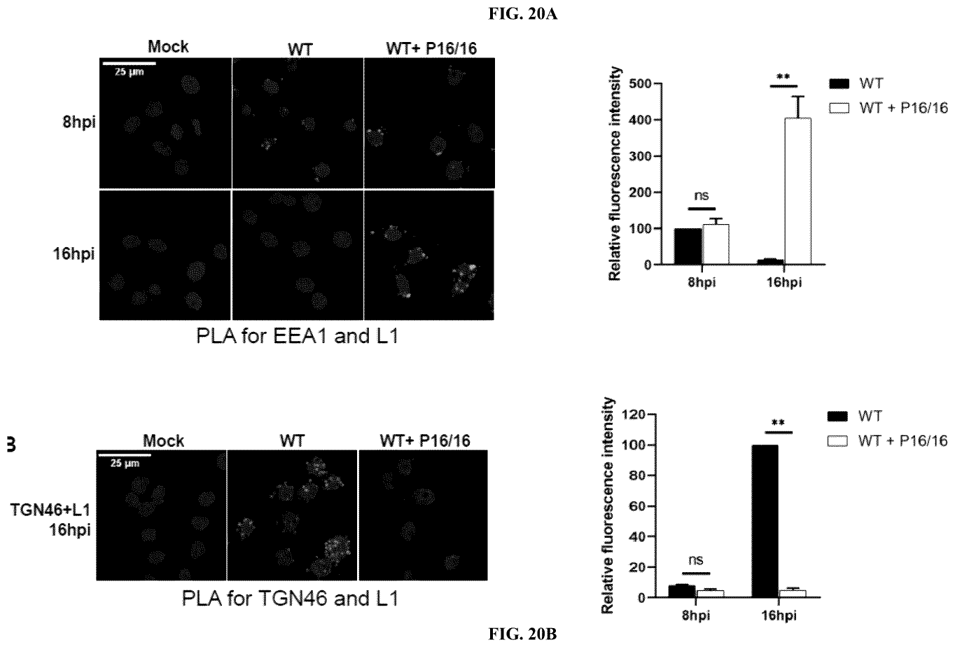

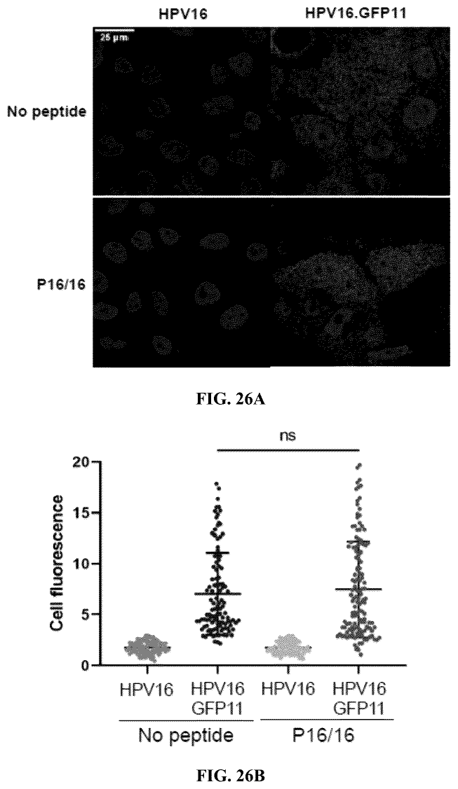

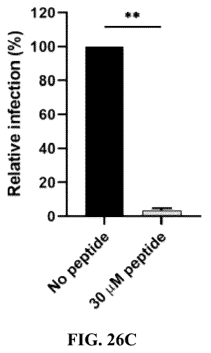

We next identified the step of infection blocked by P16/16. Immunofluorescence studies with antibodies recognizing L1 showed that P16/16 did not inhibit virus internalization (FIG. 25). We then used PLA to examine the localization of incoming HPV16 PsV in cells treated with peptide. PLA for L1 and the endosome marker EEA1 at 8 h.p.i. confirmed arrival of HPV at the endosome regardless of the presence of the peptide (FIG. 20A). However, EEA1/L1 PLA at 16 h.p.i. showed striking accumulation of the virus in the endosome in cells treated with P16/16, a time when the virus had exited the endosome in untreated cells (FIG. 20A). At 16 h.p.i, PLA for L1 and the TGN marker, TGN46, showed that P16/16 inhibited the arrival of HPV in the TGN (FIG. 20B). A split GFP fluorescence reconstitution assay showed that the peptide did not inhibit protrusion of the C terminus of the L2 protein into the cytoplasm (FIG. 26). Thus, P16/16 blocks endosome exit and inhibits intracellular HPV trafficking after protrusion of L2 into the cytoplasm.

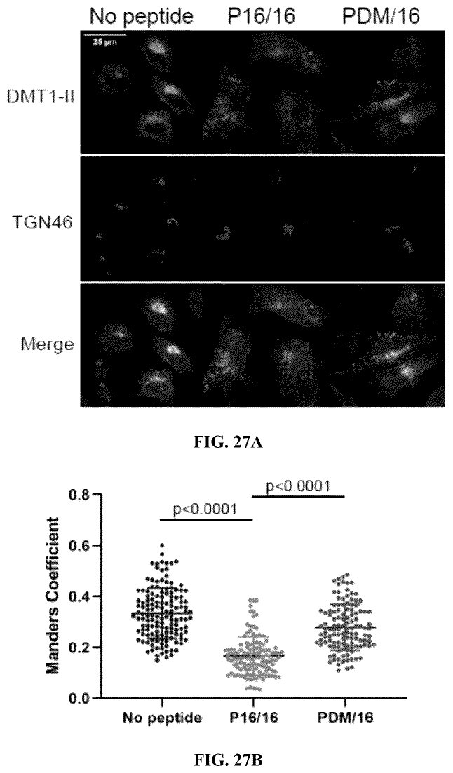

Finally, we tested if the L2 peptide inhibited retrograde transport of a cellular retromer cargo, DMT1-II, which contains a YLL RBS in its cytoplasmic domain required for retromer-mediated transport to the recycling endosome and TGN. We transfected cells with a plasmid expressing GFP fused to DMT1-II, and six hours later, cells were treated with P16/16 or PDM/16 or left untreated. The distribution of GFP fluorescence and anti-TGN46 antibody staining was assessed ˜20 hours later by confocal microscopy. As shown in FIG. 27, in untreated cells, DMT1-II and TGN46 showed a concentrated juxtanuclear pattern, with considerable overlap between TGN46 and DMT1-II staining. In contrast, treatment with the wild-type but not the mutant peptide caused DMT1-II to redistribute to a more diffuse localization with less overlap with TGN46. Neither peptide affected the distribution of TGN46. This result shows that a CPP linked to an RBS also affects trafficking of a cellular retromer cargo.

We describe the use of a CPP to deliver soluble peptides containing the HPV16 RBS into the cytoplasm where it sequestered retromer from L2 in the virion and inhibited endosome exit. It may be possible to increase the potency of inhibitory peptides by multimerization of the CPP or the RBS, use of an CPP or RBS from other sources, or other mutation or modification of the peptide sequence. The peptides did not display any obvious toxicity despite the importance of retromer in normal cell physiology, possibly because HPV infection is particularly dependent on retromer function. This might be the case, for example, if incoming HPV must travel via the retromer, whereas cellular cargo is constantly replenished by new synthesis as well as by retrograde transport.

We provide proof-of-principle for a new anti-viral strategy, namely inhibiting virus infection by treating cells with a peptide that enters cells and interferes with the association of the incoming virus with a cellular protein required for virus replication, in this case for proper intracellular trafficking of incoming virus. Because HPV is a localized infection of skin and mucous membranes, topical application of an entry inhibitor might be useful to prevent or limit genital HPV infection. Retromer has also been implicated in supporting the life cycle of other viruses including hepatitis C virus, influenza virus, and HIV, so agents that target retromer, including the peptides described here, may affect these viruses as well. These peptides may also be useful probes of retromer function in non-infected cells. More generally, these results identify intracellular virus trafficking as a potential therapeutic vulnerability

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-9. (canceled)

10. An isolated transport construct comprising a cargo molecule and a cell-penetrating peptide comprising SEQ ID NO: 360 thereof.

11. (canceled)

12. The transport construct according to claim 10, further comprising at least one selected from the group consisting of an activity modulating flanking sequence, a temperature modulating flanking sequence, and a pH modulating flanking sequence.

13. The transport construct according to claim 10, wherein the transport construct further comprises a linker connecting the cargo molecule and the cell-penetrating peptide.

14. The transport construct according to claim 10, wherein the cargo molecule is at least one selected from the group consisting of a nucleic acid; peptide; peptide-nucleic acid; protein; oligosaccharide; lipid; glycolipid; lipoprotein; small molecule compound; therapeutic drug; UV-vis, fluorescent or radioactive label; imaging agent; diagnostic agent; prophylactic agent; liposome; and virus.

15. A pharmaceutical composition comprising the transport construct according to claim 10 and at least one pharmaceutically acceptable excipient.

16-20. (canceled)


SEQUENCE LISTING (569 sequences)

SEQ ID NO: 1 - protein, 5 aa, synthetic construct
  REGION 1..5: note = cell penetrating peptide
KRRKR

SEQ ID NO: 2 - protein, 6 aa, synthetic construct
  REGION 1..6: note = cell penetrating peptide
RKRRKR

SEQ ID NO: 3 - protein, 6 aa, synthetic construct
  REGION 1..6: note = cell penetrating peptide
KRKRKR

SEQ ID NO: 4 - protein, 6 aa, synthetic construct
  REGION 1..6: note = cell penetrating peptide
RRKRKR

SEQ ID NO: 5 - protein, 6 aa, synthetic construct
  REGION 1..6: note = cell penetrating peptide
RRRRKR

SEQ ID NO: 6 - protein, 5 aa, synthetic construct
  REGION 1..5: note = cell penetrating peptide
KKRKR

SEQ ID NO: 7 - protein, 7 aa, synthetic construct
  REGION 1..7: note = cell penetrating peptide
RRRKRKR

SEQ ID NO: 8 - protein, 5 aa, synthetic construct
  REGION 1..5: note = cell penetrating peptide
RKRKR

SEQ ID NO: 9 - protein, 7 aa, synthetic construct
  REGION 1..7: note = cell penetrating peptide
KRRKRKR

SEQ ID NO: 10 - protein, 6 aa, synthetic construct
  REGION 1..6: note = cell penetrating peptide
RKKRKR

SEQ ID NO: 11 - protein, 7 aa, synthetic construct
  REGION 1..7: note = cell penetrating peptide
RKRKRKR

SEQ ID NO: 12 - protein, 7 aa, synthetic construct
  REGION 1..7: note = cell penetrating peptide
RRRRRKR

SEQ ID NO: 13 - protein, 7 aa, synthetic construct
  REGION 1..7: note = cell penetrating peptide
RKRRKRK

SEQ ID NO: 14 - protein, 5 aa, synthetic construct
  REGION 1..5: note = cell penetrating peptide
RKRRR

SEQ ID NO: 15 - protein, 7 aa, synthetic construct
  REGION 1..7: note = cell penetrating peptide
RRRRKRK

SEQ ID NO: 16 - protein, 6 aa, synthetic construct
  REGION 1..6: note = cell penetrating peptide
KKRKRK

SEQ ID NO: 17 - protein, 7 aa, synthetic construct
  REGION 1..7: note = cell penetrating peptide
RKRKKRK

SEQ ID NO: 18 - protein, 6 aa, synthetic construct
  REGION 1..6: note = cell penetrating peptide
RKRKRR

SEQ ID NO: 19 - protein, 5 aa, synthetic construct
  REGION 1..5: note = cell penetrating peptide
RKRRK

SEQ ID NO: 20 - protein, 8 aa, synthetic construct
  REGION 1..8: note = cell penetrating peptide
RRRKRKRK

SEQ ID NO: 21 - protein, 8 aa, synthetic construct
  REGION 1..8: note = cell penetrating peptide
RRRRKRKR

SEQ ID NO: 22 - protein, 7 aa, synthetic construct
  REGION 1..7: note = cell penetrating peptide
RRRRKRR

SEQ ID NO: 23 - protein, 6 aa, synthetic construct
  REGION 1..6: note = cell penetrating peptide
KRRKRK

SEQ ID NO: 24 - protein, 6 aa, synthetic construct
  REGION 1..6: note = cell penetrating peptide
KRRRKR

SEQ ID NO: 25 - protein, 7 aa, synthetic construct
  REGION 1..7: note = cell penetrating peptide
RKKRKRK

SEQ ID NO: 26 - protein, 6 aa, synthetic construct
  REGION 1..6: note = cell penetrating peptide
RKRKRK

SEQ ID NO: 27 - protein, 7 aa, synthetic construct
  REGION 1..7: note = cell penetrating peptide
RKRRRKR

SEQ ID NO: 28 - protein, 7 aa, synthetic construct
  REGION 1..7: note = cell penetrating peptide
RRKKRKR

SEQ ID NO: 29 - protein, 5 aa, synthetic construct
  REGION 1..5: note = cell penetrating peptide
RRRRK

SEQ ID NO: 30 - protein, 10 aa, synthetic construct
  REGION 1..10: note = cell penetrating peptide
RRRRRRRRRR

SEQ ID NO: 31 - protein, 5 aa, synthetic construct
  REGION 1..5: note = cell penetrating peptide
KKHKR

SEQ ID NO: 32 - protein, 5 aa, synthetic construct
  REGION 1..5: note = cell penetrating peptide
KKRKK

SEQ ID NO: 33 - protein, 7 aa, synthetic construct
  REGION 1..7: note = cell penetrating peptide
KKRKRKR

SEQ ID NO: 34 - protein, 6 aa, synthetic construct
  REGION 1..6: note = cell penetrating peptide
KRHKHK

SEQ ID NO: 35 - protein, 6 aa, synthetic construct
  REGION 1..6: note = cell penetrating peptide
KRKKRR

SEQ ID NO: 36 - protein, 8 aa, synthetic construct
  REGION 1..8: note = cell penetrating peptide
KRKRRKRK

SEQ ID NO: 37 - protein, 6 aa, synthetic construct
  REGION 1..6: note = cell penetrating peptide
KRKRRR

SEQ ID NO: 38 - protein, 8 aa, synthetic construct
  REGION 1..8: note = cell penetrating peptide
KRLKRKRK

SEQ ID NO: 39 - protein, 8 aa, synthetic construct
  REGION 1..8: note = cell penetrating peptide
KRRKRKRK

SEQ ID NO: 40 - protein, 7 aa, synthetic construct
  REGION 1..7: note = cell penetrating peptide
KRRRRKR

SEQ ID NO: 41 - protein, 6 aa, synthetic construct
  REGION 1..6: note = cell penetrating peptide
RKHKHK

SEQ ID NO: 42 - protein, 6 aa, synthetic construct
  REGION 1..6: note = cell penetrating peptide
RKKRRR

SEQ ID NO: 43 - protein, 8 aa, synthetic construct
  REGION 1..8: note = cell penetrating peptide
RKRKRKRK

SEQ ID NO: 44 - protein, 8 aa, synthetic construct
  REGION 1..8: note = cell penetrating peptide
RKRRKRKR

SEQ ID NO: 45 - protein, 8 aa, synthetic construct
  REGION 1..8: note = cell penetrating peptide
RKRRRKRK

SEQ ID NO: 46 - protein, 7 aa, synthetic construct
  REGION 1..7: note = cell penetrating peptide
RRKRKRK

SEQ ID NO: 47 - protein, 8 aa, synthetic construct
  REGION 1..8: note = cell penetrating peptide
RRKRKRKR

SEQ ID NO: 48 - protein, 5 aa, synthetic construct
  REGION 1..5: note = cell penetrating peptide
RRKRR

SEQ ID NO: 49 - protein, 8 aa, synthetic construct
  REGION 1..8: note = cell penetrating peptide
RRRRRKRK

SEQ ID NO: 50 - protein, 8 aa, synthetic construct
  REGION 1..8: note = cell penetrating peptide
RRRRRRRR

SEQ ID NO: 51 - protein, 8 aa, synthetic construct
  REGION 1..8: note = cell penetrating peptide
KGKRRKKR

SEQ ID NO: 52 - protein, 8 aa, synthetic construct
  REGION 1..8: note = cell penetrating peptide
KHRKRKRK

SEQ ID NO: 53 - protein, 7 aa, synthetic construct
  REGION 1..7: note = cell penetrating peptide
KKGKKRK

SEQ ID NO: 54 - protein, 4 aa, synthetic construct
  REGION 1..4: note = cell penetrating peptide
KKHK

SEQ ID NO: 55 - protein, 6 aa, synthetic construct
  REGION 1..6: note = cell penetrating peptide
KKHKHK

SEQ ID NO: 56 - protein, 6 aa, synthetic construct
  REGION 1..6: note = cell penetrating peptide
KKKGKK

SEQ ID NO: 57 - protein, 4 aa, synthetic construct
  REGION 1..4: note = cell penetrating peptide
KKKK

SEQ ID NO: 58 - protein, 6 aa, synthetic construct
  REGION 1..6: note = cell penetrating peptide
KKKKKR

SEQ ID NO: 59 - protein, 7 aa, synthetic construct
  REGION 1..7: note = cell penetrating peptide
KKKKKSK

SEQ ID NO: 60 - protein, 8 aa, synthetic construct
  REGION 1..8: note = cell penetrating peptide
KKKKRKRK

SEQ ID NO: 61 - protein, 8 aa, synthetic construct
  REGION 1..8: note = cell penetrating peptide
KKKKSKVR

SEQ ID NO: 62 - protein, 8 aa, synthetic construct
  REGION 1..8: note = cell penetrating peptide
KKKRKKKR

SEQ ID NO: 63 - protein, 6 aa, synthetic construct
  REGION 1..6: note = cell penetrating peptide
KKKRKR

SEQ ID NO: 64 - protein, 5 aa, synthetic construct
  REGION 1..5: note = cell penetrating peptide
KKKRR

SEQ ID NO: 65 - protein, 7 aa, synthetic construct
  REGION 1..7: note = cell penetrating peptide
KKKRRKR

SEQ ID NO: 66 - protein, 6 aa, synthetic construct
  REGION 1..6: note = cell penetrating peptide
KKKRRR

SEQ ID NO: 67 - protein, 10 aa, synthetic construct
  REGION 1..10: note = cell penetrating peptide
KKLRKKRKRR

SEQ ID NO: 68 - protein, 4 aa, synthetic construct
  REGION 1..4: note = cell penetrating peptide
KKRK

SEQ ID NO: 69 - protein, 8 aa, synthetic construct
  REGION 1..8: note = cell penetrating peptide
KKRKRKRK

SEQ ID NO: 70 - protein, 8 aa, synthetic construct
  REGION 1..8: note = cell penetrating peptide
KKRRKRKR

SEQ ID NO: 71 - protein, 5 aa, synthetic construct
  REGION 1..5: note = cell penetrating peptide
KKRRR

SEQ ID NO: 72 - protein, 8 aa, synthetic construct
  REGION 1..8: note = cell penetrating peptide
KRCRRKRR

SEQ ID NO: 73 - protein, 7 aa, synthetic construct
  REGION 1..7: note = cell penetrating peptide
KRKCKRK

SEQ ID NO: 74 - protein, 6 aa, synthetic construct
  REGION 1..6: note = cell penetrating peptide
KRKKRK

SEQ ID NO: 75 - protein, 4 aa, synthetic construct
  REGION 1..4: note = cell penetrating peptide
KRKR

SEQ ID NO: 76 - protein, 5 aa, synthetic construct
  REGION 1..5: note = cell penetrating peptide
KRKRK

SEQ ID NO: 77 - protein, 7 aa, synthetic construct
  REGION 1..7: note = cell penetrating peptide
KRKRKRK

SEQ ID NO: 78 - protein, 9 aa, synthetic construct
  REGION 1..9: note = cell penetrating peptide
KRKRKRKRK

SEQ ID NO: 79 - protein, 7 aa, synthetic construct
  REGION 1..7: note = cell penetrating peptide
KRKRKRR

SEQ ID NO: 80 - protein, 6 aa, synthetic construct
  REGION 1..6: note = cell penetrating peptide
KRKRRK

SEQ ID NO: 81 - protein, 7 aa, synthetic construct
  REGION 1..7: note = cell penetrating peptide
KRKRRKR

SEQ ID NO: 82 - protein, 8 aa, synthetic construct
  REGION 1..8: note = cell penetrating peptide
KRKRRRKR

SEQ ID NO: 83 - protein, 8 aa, synthetic construct
  REGION 1..8: note = cell penetrating peptide
KRKRRRRR

SEQ ID NO: 84 - protein, 5 aa, synthetic construct
  REGION 1..5: note = cell penetrating peptide
KRRKK

SEQ ID NO: 85 - protein, 7 aa, synthetic construct
  REGION 1..7: note = cell penetrating peptide
KRRKKRK

SEQ ID NO: 86 - protein, 4 aa, synthetic construct
  REGION 1..4: note = cell penetrating peptide
KRRR

SEQ ID NO: 87 - protein, 5 aa, synthetic construct
  REGION 1..5: note = cell penetrating peptide
KRRRK

SEQ ID NO: 88 - protein, 5 aa, synthetic construct
  REGION 1..5: note = cell penetrating peptide
KRRRR

SEQ ID NO: 89 - protein, 6 aa, synthetic construct
  REGION 1..6: note = cell penetrating peptide
KRRRRR

SEQ ID NO: 90 - protein, 7 aa, synthetic construct
  REGION 1..7: note = cell penetrating peptide
KRRRRRR

SEQ ID NO: 91 - protein, 9 aa, synthetic construct
  REGION 1..9: note = cell penetrating peptide
KRRRRRRRK

SEQ ID NO: 92 - protein, 10 aa, synthetic construct
  REGION 1..10: note = cell penetrating peptide
KRRRRRRTRR

SEQ ID NO: 93 - protein, 6 aa, synthetic construct
  REGION 1..6: note = cell penetrating peptide
KSRKRK

SEQ ID NO: 94 - protein, 8 aa, synthetic construct
  REGION 1..8: note = cell penetrating peptide
KTRKRKRK

SEQ ID NO: 95 - protein, 7 aa, synthetic construct
  REGION 1..7: note = cell penetrating peptide
KWKKRKR

SEQ ID NO: 96 - protein, 8 aa, synthetic construct
  REGION 1..8: note = cell penetrating peptide
RGRKRKRR

SEQ ID NO: 97 - protein, 7 aa, synthetic construct
  REGION 1..7: note = cell penetrating peptide
RHRRKRR

SEQ ID NO: 98 - protein, 7 aa, synthetic construct
  REGION 1..7: note = cell penetrating peptide
RHRRRKR

SEQ ID NO: 99 - protein, 8 aa, synthetic construct
  REGION 1..8: note = cell penetrating peptide
RKFKRRRK

SEQ ID NO: 100 - protein, 6 aa, synthetic construct
  REGION 1..6: note = cell penetrating peptide
RKHRHK

SEQ ID NO: 101 - protein, 6 aa, synthetic construct
  REGION 1..6: note = cell penetrating peptide
RKKKKK

SEQ ID NO: 102 - protein, 7 aa, synthetic construct
  REGION 1..7: note = cell penetrating peptide
RKKKKRK

SEQ ID NO: 103 - protein, 5 aa, synthetic construct
  REGION 1..5: note = cell penetrating peptide
RKKKR

SEQ ID NO: 104 - protein, 5 aa, synthetic construct
  REGION 1..5: note = cell penetrating peptide
RKKRK

SEQ ID NO: 105 - protein, 5 aa, synthetic construct
  REGION 1..5: note = cell penetrating peptide
RKKRR

SEQ ID NO: 106 - protein, 6 aa, synthetic construct
  REGION 1..6: note = cell penetrating peptide
RKKRRK

SEQ ID NO: 107 - protein, 7 aa, synthetic construct
  REGION 1..7: note = cell penetrating peptide
RKKRRKR

SEQ ID NO: 108 - protein, 8 aa, synthetic construct
  REGION 1..8: note = cell penetrating peptide
RKKRRKRK

SEQ ID NO: 109 - protein, 7 aa, synthetic construct
  REGION 1..7: note = cell penetrating peptide
RKKRRRR

SEQ ID NO: 110 - protein, 9 aa, synthetic construct
  REGION 1..9: note = cell penetrating peptide
RKRFKRKRK

SEQ ID NO: 111 - protein, 4 aa, synthetic construct
  REGION 1..4: note = cell penetrating peptide
RKRK

SEQ ID NO: 112 - protein, 6 aa, synthetic construct
  REGION 1..6: note = cell penetrating peptide
RKRKHK

SEQ ID NO: 113 - protein, 7 aa, synthetic construct
  REGION 1..7: note = cell penetrating peptide
RKRKKRR

SEQ ID NO: 114 - protein, 8 aa, synthetic construct
  REGION 1..8: note = cell penetrating peptide
RKRKRKYR

SEQ ID NO: 115 - protein, 8 aa, synthetic construct
  REGION 1..8: note = cell penetrating peptide
RKRKRRRK

SEQ ID NO: 116 - protein, 4 aa, synthetic construct
  REGION 1..4: note = cell penetrating peptide
RKRR

SEQ ID NO: 117 - protein, 6 aa, synthetic construct
  REGION 1..6: note = cell penetrating peptide
RKRRKK

SEQ ID NO: 118 - protein, 7 aa, synthetic construct
  REGION 1..7: note = cell penetrating peptide
RKRRKRR

SEQ ID NO: 119 - protein, 9 aa, synthetic construct
  REGION 1..9: note = cell penetrating peptide
RKRRRRRKR

SEQ ID NO: 120 - protein, 10 aa, synthetic construct
  REGION 1..10: note = cell penetrating peptide
RKRRRRRRRR

SEQ ID NO: 121 - protein, 7 aa, synthetic construct
  REGION 1..7: note = cell penetrating peptide
RLKRKRK

SEQ ID NO: 122 - protein, 8 aa, synthetic construct
  REGION 1..8: note = cell penetrating peptide
RLKRKRKR

SEQ ID NO: 123 - protein, 8 aa, synthetic construct
  REGION 1..8: note = cell penetrating peptide
RLKRRRRR

SEQ ID NO: 124 - protein, 9 aa, synthetic construct
  REGION 1..9: note = cell penetrating peptide
RLRKRRKRR

SEQ ID NO: 125 - protein, 6 aa, synthetic construct
  REGION 1..6: note = cell penetrating peptide
RLRRKR

SEQ ID NO: 126 - protein, 8 aa, synthetic construct
  REGION 1..8: note = cell penetrating peptide
RLRRKRKR

SEQ ID NO: 127 - protein, 8 aa, synthetic construct
  REGION 1..8: note = cell penetrating peptide
RRKKRKRK

SEQ ID NO: 128 - protein, 5 aa, synthetic construct
  REGION 1..5: note = cell penetrating peptide
RRKRK

SEQ ID NO: 129 - protein, 9 aa, synthetic construct
  REGION 1..9: note = cell penetrating peptide
RRKRKKRKR

SEQ ID NO: 130 - protein, 10 aa, synthetic construct
  REGION 1..10: note = cell penetrating peptide
RRKRKKRKRR

SEQ ID NO: 131 - protein, 7 aa, synthetic construct
  REGION 1..7: note = cell penetrating peptide
RRKRKRR

SEQ ID NO: 132 - protein, 9 aa, synthetic construct
  REGION 1..9: note = cell penetrating peptide
RRKRRKRKR

SEQ ID NO: 133 - protein, 8 aa, synthetic construct
  REGION 1..8: note = cell penetrating peptide
RRKRRKRR

SEQ ID NO: 134 - protein, 7 aa, synthetic construct
  REGION 1..7: note = cell penetrating peptide
RRKRRRR

SEQ ID NO: 135 - protein, 10 aa, synthetic construct
  REGION 1..10: note = cell penetrating peptide
RRKRRRRGRR

SEQ ID NO: 136 - protein, 9 aa, synthetic construct
  REGION 1..9: note = cell penetrating peptide
RRKRRRRKR

SEQ ID NO: 137 - protein, 9 aa, synthetic construct
  REGION 1..9: note = cell penetrating peptide
RRKRRRRRK

SEQ ID NO: 138 - protein, 11 aa, synthetic construct
  REGION 1..11: note = cell penetrating peptide
RRKRRRRRRKR

SEQ ID NO: 139 - protein, 8 aa, synthetic construct
  REGION 1..8: note = cell penetrating peptide
RRLRRKRK

SEQ ID NO: 140 - protein, 9 aa, synthetic construct
  REGION 1..9: note = cell penetrating peptide
RRRGRKRKR

SEQ ID NO: 141 - protein, 7 aa, synthetic construct
  REGION 1..7: note = cell penetrating peptide
RRRKRIK

SEQ ID NO: 142 - protein, 6 aa, synthetic construct
  REGION 1..6: note = cell penetrating peptide
RRRKRK

SEQ ID NO: 143 - protein, 6 aa, synthetic construct
  REGION 1..6: note = cell penetrating peptide
RRRKRR

SEQ ID NO: 144 - protein, 9 aa, synthetic construct
  REGION 1..9: note = cell penetrating peptide
RRRKRRKRR

SEQ ID NO: 145 - protein, 4 aa, synthetic construct
  REGION 1..4: note = cell penetrating peptide
RRRR

SEQ ID NO: 146 - protein, 7 aa, synthetic construct
  REGION 1..7: note = cell penetrating peptide
RRRRARR

SEQ ID NO: 147 - protein, 9 aa, synthetic construct
  REGION 1..9: note = cell penetrating peptide
RRRRKRKRR

SEQ ID NO: 148 - protein, 8 aa, synthetic construct
  REGION 1..8: note = cell penetrating peptide
RRRRKRRR

SEQ ID NO: 149 - protein, 6 aa, synthetic construct
  REGION 1..6: note = cell penetrating peptide
RRRRRK

SEQ ID NO: 150 - protein, 9 aa, synthetic construct
  REGION 1..9: note = cell penetrating peptide
RRRRRKRRR

SEQ ID NO: 151 - protein, 6 aa, synthetic construct
  REGION 1..6: note = cell penetrating peptide
RRRRRR

SEQ ID NO: 152 - protein, 8 aa, synthetic construct
  REGION 1..8: note = cell penetrating peptide
RRRRRRKR

SEQ ID NO: 153 - protein, 7 aa, synthetic construct
  REGION 1..7: note = cell penetrating peptide
RRRRRRR

SEQ ID NO: 154 - protein, 9 aa, synthetic construct
  REGION 1..9: note = cell penetrating peptide
RRRRRRRRK

SEQ ID NO: 155 - protein, 10 aa, synthetic construct
  REGION 1..10: note = cell penetrating peptide
RRRRRRRRRK

SEQ ID NO: 156 - protein, 8 aa, synthetic construct
  REGION 1..8: note = cell penetrating peptide
RSKKRKRK

SEQ ID NO: 157 - protein, 7 aa, synthetic construct
  REGION 1..7: note = cell penetrating peptide
RSRKRKR

SEQ ID NO: 158 - protein, 8 aa, synthetic construct
  REGION 1..8: note = cell penetrating peptide
RTKRRKRK

SEQ ID NO: 159 - protein, 6 aa, synthetic construct
  REGION 1..6: note = cell penetrating peptide
RVKKRR

SEQ ID NO: 160 - protein, 8 aa, synthetic construct
  REGION 1..8: note = cell penetrating peptide
RWKKRKRK

SEQ ID NO: 161 - protein, 7 aa, synthetic construct
  REGION 1..7: note = cell penetrating peptide
RWRKRKR

SEQ ID NO: 162 - protein, 8 aa, synthetic construct
  REGION 1..8: note = cell penetrating peptide
RWRKRKRK

SEQ ID NO: 163 - protein, 9 aa, synthetic construct
  REGION 1..9: note = cell penetrating peptide
RWRRKRRRR

SEQ ID NO: 164 - protein, 8 aa, synthetic construct
  REGION 1..8: note = cell penetrating peptide
RWRRRKRK

SEQ ID NO: 165 - protein, 32 aa, synthetic construct
  REGION 1..32: note = cell penetrating peptide
DGDISLTDLNYRQYFLHPSLMKGKRRKKRVSG

SEQ ID NO: 166 - protein, 34 aa, synthetic construct
  REGION 1..34: note = cell penetrating peptide
PISPPLPFDSVTATFDLHPGLKHRKRKRKHHGLT

SEQ ID NO: 167 - protein, 33 aa, synthetic construct
  REGION 1..33: note = cell penetrating peptide
LDPAILIDVASDTYYIHPSLLKKGKKRKYSDIF

SEQ ID NO: 168 - protein, 32 aa, synthetic construct
  REGION 1..32: note = cell penetrating peptide
PDIILNFEDDTATFFLHPSLLKKHKHNKHWFL

SEQ ID NO: 169 - protein, 31 aa, synthetic construct
  REGION 1..31: note = cell penetrating peptide
PAIIIDFDEDTATFFLHPSLLKKHKHKHWFF

SEQ ID NO: 170 - protein, 39 aa, synthetic construct
  REGION 1..39: note = cell penetrating peptide
PYSIYIVGSDYYLFPSYIFFPKKHKRLHYFFTDGYVAAW

SEQ ID NO: 171 - protein, 39 aa, synthetic construct
  REGION 1..39: note = cell penetrating peptide
PYAIYVVGTDFYLFPSYIFFPKKHKRIHYSFTDGYVAAW

SEQ ID NO: 172 - protein, 29 aa, synthetic construct
  REGION 1..29: note = cell penetrating peptide
ILTDSDAGFFWNTFLHPSLLSKKKGKKTF

SEQ ID NO: 173 - protein, 58 aa, synthetic construct
  REGION 1..58: note = cell penetrating peptide
QPSFGVDVYSDDFYLHPGLYHKKKKETNRIFLMFCRCPYGCRPLENCIFHLKSQLLKC

SEQ ID NO: 174 - protein, 43 aa, synthetic construct
  REGION 1..43: note = cell penetrating peptide
TPAVVIDLLGGTDFYLHPALFKKKKKRLFCDFFADGGVASCTE

SEQ ID NO: 175 - protein, 44 aa, synthetic construct
  REGION 1..44: note = cell penetrating peptide
PSTANSLDASYSYYLHPSLNNKKKKKSKGLRGGWWFVADDLLAT

SEQ ID NO: 176 - protein, 31 aa, synthetic construct
  REGION 1..31: note = cell penetrating peptide
TPTVVIRFGEAGTDYYLHPSLKKKKRKRKYL

SEQ ID NO: 177 - protein, 34 aa, synthetic construct
  REGION 1..34: note = cell penetrating peptide
GIVIDLSDDYYRHYYLHPSLLKKKKSKVRKLWYA

SEQ ID NO: 178 - protein, 31 aa, synthetic construct
  REGION 1..31: note = cell penetrating peptide
IPVSPAVSLGGANYWLEPSLIKKKRKKKRLI

SEQ ID NO: 179 - protein, 31 aa, synthetic construct
  REGION 1..31: note = cell penetrating peptide
QPPTVILDLFSDDYFLHPSYLKKKRKRSDIF

SEQ ID NO: 180 - protein, 38 aa, synthetic construct
  REGION 1..38: note = cell penetrating peptide
TILIDFQSSYGDFFLHPSLIPKKKRRLGLFTDEYVVTE

SEQ ID NO: 181 - protein, 33 aa, synthetic construct
  REGION 1..33: note = cell penetrating peptide
TPVIVLDWQQSNDFFLHPSLIKKKRRKRSAAFF

SEQ ID NO: 182 - protein, 31 aa, synthetic construct
  REGION 1..31: note = cell penetrating peptide
IGPSYYVGVDNDFYLHPSLIPKKKRRRLDYF

SEQ ID NO: 183 - protein, 33 aa, synthetic construct
  REGION 1..33: note = cell penetrating peptide
PIRPGLDVYDSIDFYLHPSLGKKLRKKRKRRFY

SEQ ID NO: 184 - protein, 38 aa, synthetic construct
  REGION 1..38: note = cell penetrating peptide
TFAITIYGSNYYLLPLLFFLLKKRKHLPYFFTDGIVAS

SEQ ID NO: 185 - protein, 29 aa, synthetic construct
  REGION 1..29: note = cell penetrating peptide
PWVVDGDGGSGYWIDPSLLTNKKRKKHFH

SEQ ID NO: 186 - protein, 37 aa, synthetic construct
  REGION 1..37: note = cell penetrating peptide
VHPAYSVTFSMLSELDDPFLTKKRKKCFADGCLDTFY

SEQ ID NO: 187 - protein, 38 aa, synthetic construct
  REGION 1..38: note = cell penetrating peptide
TQYIGIHGTHYYLWPLYYFIPKKRKRVPYFFADGFVAA

SEQ ID NO: 188 - protein, 38 aa, synthetic construct
  REGION 1..38: note = cell penetrating peptide
TYAITIQGSNYYLLPLLYFFLKKRKRIPYFFSDGYVAV

SEQ ID NO: 189 - protein, 38 aa, synthetic construct
  REGION 1..38: note = cell penetrating peptide
TTYIGIHGTQYYLWPWYYYFPKKRKRIPYFFADGFVAA

SEQ ID NO: 190 - protein, 38 aa, synthetic construct
  REGION 1..38: note = cell penetrating peptide
TVAIAIQGSNYYLLPLLYYFLKKRKRIPYFFTDGFVAV

SEQ ID NO: 191 - protein, 38 aa, synthetic construct
  REGION 1..38: note = cell penetrating peptide
PSSFIVYGTEYYLMPSYIFFPKKRKRVHYFFADGFVAA

SEQ ID NO: 192 - protein, 39 aa, synthetic construct
  REGION 1..39: note = cell penetrating peptide
IQSVFIDGTDYYLLPNYIFFPKKRKRVHYSFADGYVAAW

SEQ ID NO: 193 - protein, 39 aa, synthetic construct
  REGION 1..39: note = cell penetrating peptide
PYAIYILGSDYYLLPNYIFFPKKRKRVPYSFSDGFVAAW

SEQ ID NO: 194 - protein, 38 aa, synthetic construct
  REGION 1..38: note = cell penetrating peptide
THSVVLQGTNYYLWPNYYFIFKKRKRVPYFLTDGFVAF

SEQ ID NO: 195 - protein, 38 aa, synthetic construct
  REGION 1..38: note = cell penetrating peptide
PGSVLVNGSTYYLLPPLGLLPKKRKRFPYFFADGNVEA

SEQ ID NO: 196 - protein, 38 aa, synthetic construct
  REGION 1..38: note = cell penetrating peptide
THYIGINGTHYYLWPLYYFLPKKRKRVPYFFADGLLAA

SEQ ID NO: 197 - protein, 39 aa, synthetic construct
  REGION 1..39: note = cell penetrating peptide
SQSVFVDGTDYFLLPNYLFFPKKRKRVHYSFADGFVATW

SEQ ID NO: 198 - protein, 30 aa, synthetic construct
  REGION 1..30: note = cell penetrating peptide
TIVIEAFDSSGGFYLHPSFVGKKRKRLYFL

SEQ ID NO: 199 - protein, 43 aa, synthetic construct
  REGION 1..43: note = cell penetrating peptide
VPIIVVDATSQDFYLHPYLQHKKRKRKHFVYMFADGSVASEYQ

SEQ ID NO: 200 - protein, 32 aa, synthetic construct
  REGION 1..32: note = cell penetrating peptide
VAPAVHVEAFGSNYYLHPSLLKKRKRKYLDVF

SEQ ID NO: 201 - protein, 31 aa, synthetic construct
  REGION 1..31: note = cell penetrating peptide
PAIVIDILDSSADYYLHPSLIKKRKRKHFFF

SEQ ID NO: 202 - protein, 31 aa, synthetic construct
  REGION 1..31: note = cell penetrating peptide
PAILIEIWDSGSNYSLHPSLLKKRKRKLLFL

SEQ ID NO: 203 - protein, 31 aa, synthetic construct
  REGION 1..31: note = cell penetrating peptide
LPAVVIHIYDDSGDFYLHPSLKKRKRKRAYL

SEQ ID NO: 204 - protein, 33 aa, synthetic construct
  REGION 1..33: note = cell penetrating peptide
GPSILIDEFSSEDFVLHPSLSKKRKRKRLYSDF

SEQ ID NO: 205 - protein, 31 aa, synthetic construct
  REGION 1..31: note = cell penetrating peptide
TPTVVIKFAEAGGRFLFTPSLKKRKRKRKYL

SEQ ID NO: 206 - protein, 33 aa, synthetic construct
  REGION 1..33: note = cell penetrating peptide
PFIPSTLQFDTFDYDIHPSAIKKRRKRKRSDFM

SEQ ID NO: 207 - protein, 30 aa, synthetic construct
  REGION 1..30: note = cell penetrating peptide
VAPSYIPDYYADFYLYPSFLPKKRRRIDIV

SEQ ID NO: 208 - protein, 57 aa, synthetic construct
  REGION 1..57: note = cell penetrating peptide
SATPPTSTDTAGFSMLDPSLLKRCRRKRRSCFADGIVDARQSEVLSAPCSCDKGAQY

SEQ ID NO: 209 - protein, 31 aa, synthetic construct
  REGION 1..31: note = cell penetrating peptide
PDIIITFEEGTATFFLHPSLLKRHKHKHWFF

SEQ ID NO: 210 - protein, 31 aa, synthetic construct
  REGION 1..31: note = cell penetrating peptide
PDIVIDMEEDTATFTLHPSLLKRHKHKHWFF

SEQ ID NO: 211 - protein, 32 aa, synthetic construct
  REGION 1..32: note = cell penetrating peptide
PTVVIHIHDTSGDYYLHPSLQKRKCKRKHRYL

SEQ ID NO: 212 - protein, 31 aa, synthetic construct
  REGION 1..31: note = cell penetrating peptide
GPTSPVSFYSDDFYLHPSLFTKRKKRKYYNF

SEQ ID NO: 213 - protein, 29 aa, synthetic construct
  REGION 1..29: note = cell penetrating peptide
ITDSGVDGTYFLNTYLHPSLHKRKKRRFS

SEQ ID NO: 214 - protein, 29 aa, synthetic construct
  REGION 1..29: note = cell penetrating peptide
IIDFSVEGTYFLNTYAHPSLHKRKKRRLS

SEQ ID NO: 215 - protein, 37 aa, synthetic construct
  REGION 1..37: note = cell penetrating peptide
TPIITGVFVSFDFWLHPSQLLKRKRSPFYLADGIVAA

SEQ ID NO: 216 - protein, 30 aa, synthetic construct
  REGION 1..30: note = cell penetrating peptide
PYAPSPITVFGDTFYLNPSLLKRKRKQYFY

SEQ ID NO: 217 - protein, 38 aa, synthetic construct
  REGION 1..38: note = cell penetrating peptide
PAGPIYIYGSGFILHPSYYLLKRKRKRLSYSFTDVATY

SEQ ID NO: 218 - protein, 30 aa, synthetic construct
  REGION 1..30: note = cell penetrating peptide
PAVVIHIADASGDFYLHPSLQKRKRKRAYL

SEQ ID NO: 219 - protein, 30 aa, synthetic construct
  REGION 1..30: note = cell penetrating peptide
EPAIIIDLESASDFFIHPSLLKRKRKRPLL

SEQ ID NO: 220 - protein, 30 aa, synthetic construct
  REGION 1..30: note = cell penetrating peptide
EPAIIIDLESANDFLIHPSLLKRKRKRPLL

SEQ ID NO: 221 - protein, 30 aa, synthetic construct
  REGION 1..30: note = cell penetrating peptide
EPAIVIDLESASDFFIHPSLLKRKRKRPLL

SEQ ID NO: 222 - protein, 30 aa, synthetic construct
  REGION 1..30: note = cell penetrating peptide
INPSLGASVIFDTYDLHPSLLKRKRKRPFF

SEQ ID NO: 223 - protein, 30 aa, synthetic construct
  REGION 1..30: note = cell penetrating peptide
EPAIIIDVDSATDFLIHPSLLKRKRKRHLL

SEQ ID NO: 224 - protein, 31 aa, synthetic construct
  REGION 1..31: note = cell penetrating peptide
PLYPTEVTVYGDSFVIDPFFLKRKRKRYTLY

SEQ ID NO: 225 - protein, 31 aa, synthetic construct
  REGION 1..31: note = cell penetrating peptide
TEPHLIIDLFSDDFYLHPGYLKRKRKRSDIF

SEQ ID NO: 226 - protein, 31 aa, synthetic construct
  REGION 1..31: note = cell penetrating peptide
IMPGVTIDIYSIDYDIHPSLLKRKRKRIDYV

SEQ ID NO: 227 - protein, 30 aa, synthetic construct
  REGION 1..30: note = cell penetrating peptide
EPAIIIDLESATDFLIHPSLLKRKRKRNLL

SEQ ID NO: 228 - protein, 30 aa, synthetic construct
  REGION 1..30: note = cell penetrating peptide
AIVIEIWGSGNSYSLHPSLLSKRKRKRLSL

SEQ ID NO: 229 - protein, 37 aa, synthetic construct
  REGION 1..37: note = cell penetrating peptide
HIFVYSPHLTSFDFLPHPSLLKRKRKRSLDDDFTILQ

SEQ ID NO: 230 - protein, 32 aa, synthetic construct
  REGION 1..32: note = cell penetrating peptide
IIDNELAVWFHSYFLHPSKLGKRKRKRSDSSV

SEQ ID NO: 231 - protein, 30 aa, synthetic construct
  REGION 1..30: note = cell penetrating peptide
IIDGNTIDLYSSNFTLHPSLLKRKRKRKHA

SEQ ID NO: 232 - protein, 32 aa, synthetic construct
  REGION 1..32: note = cell penetrating peptide
PTVVIHISDTSGDYYLHPSLQKRKRKRKRKYL

SEQ ID NO: 233 - protein, 30 aa, synthetic construct
  REGION 1..30: note = cell penetrating peptide
PTIIVTKETGVTYDLHPSLFGKRKRKRRSL

SEQ ID NO: 234 - protein, 31 aa, synthetic construct
  REGION 1..31: note = cell penetrating peptide
TVLINVYDEGLDFLLHPSMFPKRKRRKLAFL

SEQ ID NO: 235 - protein, 31 aa, synthetic construct
  REGION 1..31: note = cell penetrating peptide
PGILIEVLDSSGDFYLHPSLLKRKRRKRPSF

SEQ ID NO: 236 - protein, 31 aa, synthetic construct
  REGION 1..31: note = cell penetrating peptide
PTVVIHIADTSGDYYLHPSLQKRKRRKRKYL

SEQ ID NO: 237 - protein, 31 aa, synthetic construct
  REGION 1..31: note = cell penetrating peptide
PVVIIYPHDNTGDFYLHPSLHKRKRRKRKYF

SEQ ID NO: 238 - protein, 31 aa, synthetic construct
  REGION 1..31: note = cell penetrating peptide
PAPHTVSDINDDYYLYPSLYPKRKRRRLDFF

SEQ ID NO: 239 - protein, 31 aa, synthetic construct
  REGION 1..31: note = cell penetrating peptide
PLEPANAIEVYYDFFLHPALQKRKRRRLDVF

SEQ ID NO: 240 - protein, 32 aa, synthetic construct
  REGION 1..32: note = cell penetrating peptide
DGNINLQEVLYRQYFLHPSLMKRKRRRKRLFG

SEQ ID NO: 241 - protein, 33 aa, synthetic construct
  REGION 1..33: note = cell penetrating peptide
RPPIYIGSSPGVDYYLHPSLYKRKRRRRRHSYL

SEQ ID NO: 242 - protein, 31 aa, synthetic construct
  REGION 1..31: note = cell penetrating peptide
PVVIIHTYDTSGDFYLHPSLTKRLKRKRKYL

SEQ ID NO: 243 - protein, 31 aa, synthetic construct
  REGION 1..31: note = cell penetrating peptide
PVVIIHTYDTSGDFYLHPSLHKRLKRKRKYL

SEQ ID NO: 244 - protein, 37 aa, synthetic construct
  REGION 1..37: note = cell penetrating peptide
STPLYPPRHVFFSDLDDPIMFKRRKKCFADGCVDAFY

SEQ ID NO: 245 - protein, 32 aa, synthetic construct
  REGION 1..32: note = cell penetrating peptide
PPTVIVYDYDDSVDFYLHPSLKRRKKRKYIVY

SEQ ID NO: 246 - protein, 38 aa, synthetic construct
  REGION 1..38: note = cell penetrating peptide
PSSVYIFGGDYYLMPSYVLWPKRRKRVHYFFADGFVAA

SEQ ID NO: 247 - protein, 38 aa, synthetic construct
  REGION 1..38: note = cell penetrating peptide
LPAIVVHGDNYYLWPYIYLIHKRRKRMPYFFSDGFVAY

SEQ ID NO: 248 - protein, 38 aa, synthetic construct
  REGION 1..38: note = cell penetrating peptide
PSSVYIFGGDYYLLPSYILWPKRRKRVNYFFADGFVAA

SEQ ID NO: 249 - protein, 38 aa, synthetic construct
  REGION 1..38: note = cell penetrating peptide
PSSVYIVGGDYYLLPSYVLWPKRRKRVHYFFADGYVAA

SEQ ID NO: 250 - protein, 39 aa, synthetic construct
  REGION 1..39: note = cell penetrating peptide
PHSIYIQGSDFYLLPAYVFFPKRRKRVPYSFSDGFVAAW

SEQ ID NO: 251 - protein, 39 aa, synthetic construct
  REGION 1..39: note = cell penetrating peptide
PHSIYVEGFDFYLLPAYIFFPKRRKRVPYSFADGFVAAW

SEQ ID NO: 252 - protein, 39 aa, synthetic construct
  REGION 1..39: note = cell penetrating peptide
PYSVYIQGSDYYLLPNYIFFPKRRKRVPYSFSDGFVAAW

SEQ ID NO: 253 - protein, 38 aa, synthetic construct
  REGION 1..38: note = cell penetrating peptide
PSAVSIYGTDFYLHPSLLHFGKRRKRISYFFADNYVAA

SEQ ID NO: 254 - protein, 39 aa, synthetic construct
  REGION 1..39: note = cell penetrating peptide
PYAIYIVGSDYYLLPNYIFFPKRRKRVPYSFSDGFVAAW

SEQ ID NO: 255 - protein, 38 aa, synthetic construct
  REGION 1..38: note = cell penetrating peptide
QHSIYVHGTDFYLLPGYLFVPKRRKRFIYSFADGYVAA

SEQ ID NO: 256 - protein, 39 aa, synthetic construct
  REGION 1..39: note = cell penetrating peptide
QPSAAISIFASDFYLHPSYILKRRKRVPYTFFADGIVAS

SEQ ID NO: 257 - protein, 38 aa, synthetic construct
  REGION 1..38: note = cell penetrating peptide
APPASVTVVSGDFVLHPSYFWKRRKRVSYFFADGVVAA

SEQ ID NO: 258 - protein, 38 aa, synthetic construct
  REGION 1..38: note = cell penetrating peptide
VIPAAVTIYAGDFFLHPSYIWKRRKRVSYFLADGIVAA

SEQ ID NO: 259 - protein, 29 aa, synthetic construct
  REGION 1..29: note = cell penetrating peptide
PVIVIHGVYFSVDFYLHPHLLKRRKRFHF

SEQ ID NO: 260 - protein, 38 aa, synthetic construct
  REGION 1..38: note = cell penetrating peptide
PQSVSIHGTDFYLHPSLWHLGKRRKRFSYFFTDNYVAA

SEQ ID NO: 261 - protein, 38 aa, synthetic construct
  REGION 1..38: note = cell penetrating peptide
IGHVVVHGGDFYLHPSYYTLHKRRKRMPRFLADVSVAA

SEQ ID NO: 262 - protein, 39 aa, synthetic construct
  REGION 1..39: note = cell penetrating peptide
PQAIYIQGTDFYLVPNYVFFPKRRKRVPYSFADGFVAAW

SEQ ID NO: 263 - protein, 32 aa, synthetic construct
  REGION 1..32: note = cell penetrating peptide
YTPVIYIDPFGSDFYLHPALLKRRKRKYSEIF

SEQ ID NO: 264 - protein, 32 aa, synthetic construct
  REGION 1..32: note = cell penetrating peptide
LIPGRSVDVYSNDFIIHPSVLKRRKRKLSDSF

SEQ ID NO: 265 - protein, 32 aa, synthetic construct
  REGION 1..32: note = cell penetrating peptide
ISPAVLIDAFSADYYLHPSLMKRRKRKYSEIF

SEQ ID NO: 266 - protein, 31 aa, synthetic construct
  REGION 1..31: note = cell penetrating peptide
TPTVIIHTEDYSGDYYLHPSLKRRKRKRAYL

SEQ ID NO: 267 - protein, 31 aa, synthetic construct
  REGION 1..31: note = cell penetrating peptide
PVGPSSSVDIFYYDYDLHPSLKRRKRKRNMF

SEQ ID NO: 268 - protein, 31 aa, synthetic construct
  REGION 1..31: note = cell penetrating peptide
PIGPSNSVEIFYYDYDLHPSLKRRKRKRNVF

SEQ ID NO: 269 - protein, 31 aa, synthetic construct
  REGION 1..31: note = cell penetrating peptide
PAIILEIWGSGENYSLHPSLLKRRKRKRLIL

SEQ ID NO: 270 - protein, 31 aa, synthetic construct
  REGION 1..31: note = cell penetrating peptide
DVPSSSSDSVSSTFDLHPSLLKRRKRKRGDI

SEQ ID NO: 271 - protein, 45 aa, synthetic construct
  REGION 1..45: note = cell penetrating peptide
LEPPILLDYLGSGYYLHPSLWKRRKRKRSDTYNSFTDGIVDATEW

SEQ ID NO: 272 - protein, 31 aa, synthetic construct
  REGION 1..31: note = cell penetrating peptide
TPTVVINFEDAGGDYYLHPSLKRRKRKRKYL

SEQ ID NO: 273 - protein, 34 aa, synthetic construct
  REGION 1..34: note = cell penetrating peptide
ITPAVYLDAFSSDYYLHPALMKRRKRKRKYLEVF

SEQ ID NO: 274 - protein, 29 aa, synthetic construct
  REGION 1..29: note = cell penetrating peptide
SEQPLFVLDYSDYDLHPGLLPKRRRIDYF

SEQ ID NO: 275 - protein, 29 aa, synthetic construct
  REGION 1..29: note = cell penetrating peptide
IFSTPYYEYNYILDPSILFLLKRRRKLFV

SEQ ID NO: 276 - protein, 39 aa, synthetic construct
  REGION 1..39: note = cell penetrating peptide
PQVSIFVDGGDFYLHPSYYMLKRRRKRVSYFFTDVSVAA

SEQ ID NO: 277 - protein, 39 aa, synthetic construct
  REGION 1..39: note = cell penetrating peptide
PIYSIIADGGDFYLHPSYYLLKRRRKRIPYFFADVSVAV

SEQ ID NO: 278 - protein, 39 aa, synthetic construct
  REGION 1..39: note = cell penetrating peptide
SHSVVAQGGNYYLWPYIYLIHKRRRKRVPCFFSDGLAAY

SEQ ID NO: 279 - protein, 41 aa, synthetic construct
  REGION 1..41: note = cell penetrating peptide
PSFDWPMTDLDADFVLHPSLLKRRRRFYWSFADGGLASRTK

SEQ ID NO: 280 - protein, 40 aa, synthetic construct
  REGION 1..40: note = cell penetrating peptide
DTTHDVVIQGSTFALWPVYFLKRRRRKRIPYFLADGGVAA

SEQ ID NO: 281 - protein, 40 aa, synthetic construct
  REGION 1..40: note = cell penetrating peptide
DVTHDVYIQGATFALWPVYFFKRRRRKRIPYFFADGDVAA

SEQ ID NO: 282 - protein, 40 aa, synthetic construct
  REGION 1..40: note = cell penetrating peptide
PIVIIDLTSTSIDYFLHPSLAKRRRRRAHWSFLADVGLAT

SEQ ID NO: 283 - protein, 34 aa, synthetic construct
  REGION 1..34: note = cell penetrating peptide
PDIIIDFLTPGDTFYLHPSHFKRRRRRRYQLFFF

SEQ ID NO: 284 - protein, 32 aa, synthetic construct
  REGION 1..32: note = cell penetrating peptide
VIVDVERGSGSDYYLHPSLSLKRRRRRRRKSL

SEQ ID NO: 285 - protein, 35 aa, synthetic construct
  REGION 1..35: note = cell penetrating peptide
ITAIPAGGQSIDFLLHPGLFPKRRRRRRTRRHSYL

SEQ ID NO: 286 - protein, 32 aa, synthetic construct
  REGION 1..32: note = cell penetrating peptide
ITPLIQIDPFGPDFYLHPALMKSRKRKYLEVF

SEQ ID NO: 287 - protein, 31 aa, synthetic construct
  REGION 1..31: note = cell penetrating peptide
TPTVVINFEEAGGDYYLHPSLKTRKRKRKYL

SEQ ID NO: 288 - protein, 31 aa, synthetic construct
  REGION 1..31: note = cell penetrating peptide
TPTVIIHTEDFSGDYYLHPSLKWKKRKRAYL

SEQ ID NO: 289 - protein, 44 aa, synthetic construct
  REGION 1..44: note = cell penetrating peptide
PQPPTYEGPSSGVTYYLHPSLRGRKRKRRNLHVRFSIPDGILAS

SEQ ID NO: 290 - protein, 32 aa, synthetic construct
  REGION 1..32: note = cell penetrating peptide
VMPPIIFDPETSTFDLHPSLHRHRRKRRHIGL

SEQ ID NO: 291 - protein, 40 aa, synthetic construct
  REGION 1..40: note = cell penetrating peptide
THYVYIDGGDFYLWPVTFHFSRHRRRKRVSYFFADGTLAL

SEQ ID NO: 292 - protein, 31 aa, synthetic construct
  REGION 1..31: note = cell penetrating peptide
PVVIIHTFDTSGDFYLHPSLSRKFKRRRKYL

SEQ ID NO: 293 - protein, 31 aa, synthetic construct
  REGION 1..31: note = cell penetrating peptide
PTVIIDFEDGSATFFLHPSLLRKHKHKHWFF

SEQ ID NO: 294 - protein, 31 aa, synthetic construct
  REGION 1..31: note = cell penetrating peptide
PEIVIDFEENTATFYLHPSLLRKHKHKHWFF

SEQ ID NO: 295 - protein, 31 aa, synthetic construct
  REGION 1..31: note = cell penetrating peptide
PDIIVNLEENTATFFLHPSLLRKHRHKHWFF

SEQ ID NO: 296 - protein, 31 aa, synthetic construct
  REGION 1..31: note = cell penetrating peptide
PLVIIDPVGTGANYFLHPSLLRKKKKKLIFH

SEQ ID NO: 297 - protein, 31 aa, synthetic construct
  REGION 1..31: note = cell penetrating peptide
EVVIVQEGNNSGTFYLHPSLLRKKKKRKYVF

SEQ ID NO: 298 - protein, 38 aa, synthetic construct
  REGION 1..38: note = cell penetrating peptide
WYPTILSELSSSDFIFHPSLWRKKKRFPFFLSDGIVAA

SEQ ID NO: 299 - protein, 40 aa, synthetic construct
  REGION 1..40: note = cell penetrating peptide
VIVEDDTAAYDYWFDLYLHLPRKKRKWCSFCSLTDGIVDT

SEQ ID NO: 300 - protein, 39 aa, synthetic construct
  REGION 1..39: note = cell penetrating peptide
PTTHVVVYGGDFYLHPSYFPVRKKRKRVHRFLSDVIVAA

SEQ ID NO: 301 - protein, 31 aa, synthetic construct
  REGION 1..31: note = cell penetrating peptide
LEPALLSDIFSTDFVYRPSLYRKKRKRLEMF

SEQ ID NO: 302 - protein, 31 aa, synthetic construct
  REGION 1..31: note = cell penetrating peptide
LEPPFFSEFYSSDFVYRPSLYRKKRKRSDIF

SEQ ID NO: 303 - protein, 31 aa, synthetic construct
  REGION 1..31: note = cell penetrating peptide
PLKPALLTDFYSDFTYYPSLYRKKRKRSDLF

SEQ ID NO: 304 - protein, 30 aa, synthetic construct
  REGION 1..30: note = cell penetrating peptide
PLIVLFEPGFGPSFYLHPSLLRKKRKRVFY

SEQ ID NO: 305 - protein, 30 aa, synthetic construct
  REGION 1..30: note = cell penetrating peptide
PFIFFLFSHGDPSFFLHPSLLRKKRKRVFY

SEQ ID NO: 306 - protein, 33 aa, synthetic construct
  REGION 1..33: note = cell penetrating peptide
RPSILIDDFASNDFVLHPSLNRKKRKRKQVHFL

SEQ ID NO: 307 - protein, 30 aa, synthetic construct
  REGION 1..30: note = cell penetrating peptide
PTVVIHINDTSGDYYLHPSLQRKKRKRKYL

SEQ ID NO: 308 - protein, 42 aa, synthetic construct
  REGION 1..42: note = cell penetrating peptide
VGPSYVESTDSFPYWLAPSLQRKKRKRKTVSFCSLADGCMDS

SEQ ID NO: 309 - protein, 30 aa, synthetic construct
  REGION 1..30: note = cell penetrating peptide
INWFPLFDSYSDFALDPFFIPRKKRRLDIL

SEQ ID NO: 310 - protein, 42 aa, synthetic construct
  REGION 1..42: note = cell penetrating peptide
ISPVFIFEGNADGTYYLEEPLRKKRRKSIFLLADGSVAVYAE

SEQ ID NO: 311 - protein, 31 aa, synthetic construct
  REGION 1..31: note = cell penetrating peptide
PAISFDIYGDGLNFYLHPSLLRKKRRKRYFY

SEQ ID NO: 312 - protein, 31 aa, synthetic construct
  REGION 1..31: note = cell penetrating peptide
APAVVIHTLDKSFDYYLHPSLRKKRRKRKYL

SEQ ID NO: 313 - protein, 31 aa, synthetic construct
  REGION 1..31: note = cell penetrating peptide
TPAFYLDIYNDFNLHPALLPPRKKRRRLDIF

SEQ ID NO: 314 - protein, 31 aa, synthetic construct
  REGION 1..31: note = cell penetrating peptide
LTPSIITSSYNNFYLEPFYVPRKKRRRLDMF

SEQ ID NO: 315 - protein, 30 aa, synthetic construct
  REGION 1..30: note = cell penetrating peptide
PNINVYSLDDTGGQFRFWHFLRKKRRRRYL

SEQ ID NO: 316 - protein, 32 aa, synthetic construct
  REGION 1..32: note = cell penetrating peptide
LPVVIIHTYDTSGDFYLHPSLRKRFKRKRKYL

SEQ ID NO: 317 - protein, 30 aa, synthetic construct
  REGION 1..30: note = cell penetrating peptide
VVVISYTADVSIFSLFEPSLYRKRKYSYLY

SEQ ID NO: 318 - protein, 31 aa, synthetic construct
  REGION 1..31: note = cell penetrating peptide
PEIVIDFGESTASFSLHPSLLRKRKHKHWFF

SEQ ID NO: 319 - protein, 30 aa, synthetic construct
  REGION 1..30: note = cell penetrating peptide
IIDGHTVDLYSSNYTLHPSLLRKRKKRKHA

SEQ ID NO: 320 - protein, 30 aa, synthetic construct
  REGION 1..30: note = cell penetrating peptide
IIDGHTVDLYSNNYSLHPSLLRKRKKRKHA

SEQ ID NO: 321 - protein, 30 aa, synthetic construct
  REGION 1..30: note = cell penetrating peptide
IIDGHTIDLYSNNYSLHPSLLRKRKKRKHA

SEQ ID NO: 322 - protein, 30 aa, synthetic construct
  REGION 1..30: note = cell penetrating peptide
IIDGHIVDLYSRNYSLHPSLYRKRKKRKHA

SEQ ID NO: 323 - protein, 30 aa, synthetic construct
  REGION 1..30: note = cell penetrating peptide
DYSYDQSAGPSFTLDPSLLQLRKRKKRRYY

SEQ ID NO: 324 - protein, 37 aa, synthetic construct
  REGION 1..37: note = cell penetrating peptide
SGPIYIYGSDFILHPSLYVIPRKRKRLSYFFADVATY

SEQ ID NO: 325 - protein, 38 aa, synthetic construct
  REGION 1..38: note = cell penetrating peptide
TGDVLVHGSTYYLLPSYVLLPRKRKRFPSFFADGIVEA

SEQ ID NO: 326 - protein, 38 aa, synthetic construct
  REGION 1..38: note = cell penetrating peptide
IQSINIEGTNYFLWPIYYFLPRKRKRVPYFFTDGSMAF

SEQ ID NO: 327 - protein, 39 aa, synthetic construct
  REGION 1..39: note = cell penetrating peptide
PYAIYIQGSDYYLLPNYIFFPRKRKRVHYSFSDGFVAAW

SEQ ID NO: 328 - protein, 29 aa, synthetic construct
  REGION 1..29: note = cell penetrating peptide
GNINVSMEYFRHYYLHPSLLGRKRKRLFG

SEQ ID NO: 329 - protein, 30 aa, synthetic construct
  REGION 1..30: note = cell penetrating peptide
PSVVVDSFVTSTTFYLHPGLSRKRKRSHMF

SEQ ID NO: 330 - protein, 29 aa, synthetic construct
  REGION 1..29: note = cell penetrating peptide
QPSFAIDVDNSTFDIHPSLLPRKRKRPSF

SEQ ID NO: 331 - protein, 29 aa, synthetic construct
  REGION 1..29: note = cell penetrating peptide
LGPAVGLTVNFDDFDLHPSLLRKRKRSIL

SEQ ID NO: 332 - protein, 29 aa, synthetic construct
  REGION 1..29: note = cell penetrating peptide
LGPSQGLTVDFYDFDLHPSLLRKRKRSIF

SEQ ID NO: 333 - protein, 28 aa, synthetic construct
  REGION 1..28: note = cell penetrating peptide
PLITIRIDNSTGDYDVDPSLLRKRKRVF

SEQ ID NO: 334 - protein, 30 aa, synthetic construct
  REGION 1..30: note = cell penetrating peptide
ISPAFWNMSYSTFYLHPGYLARKRKRKVLT

SEQ ID NO: 335 - protein, 31 aa, synthetic construct
  REGION 1..31: note = cell penetrating peptide
IPARTVIYFDDFADFYLHPSLRKRKRKHVIR

SEQ ID NO: 336 - protein, 31 aa, synthetic construct
  REGION 1..31: note = cell penetrating peptide
PGADVVVVPEDSVDYYLHPSLRKRKRKYVFH

SEQ ID NO: 337 - protein, 31 aa, synthetic construct
  REGION 1..31: note = cell penetrating peptide
IPLSSPDYGSVTFDLHPGLIHRKRKRKRGSV

SEQ ID NO: 338 - protein, 42 aa, synthetic construct
  REGION 1..42: note = cell penetrating peptide
PFEPPTEPDPAETGGNFEPSLRKRKRKRVEVASRTYSLRRKR

SEQ ID NO: 339 - protein, 32 aa, synthetic construct
  REGION 1..32: note = cell penetrating peptide
TPSILVDGFSSTDFILHPSHTRKRKRKRSDYF

SEQ ID NO: 340 - protein, 32 aa, synthetic construct
  REGION 1..32: note = cell penetrating peptide
DNTPLLVLDLTSDDYFLHPSLRKRKRKRSDVF

SEQ ID NO: 341 - protein, 31 aa, synthetic construct
  REGION 1..31: note = cell penetrating peptide
PAIVIEVWASGATYSLHPSLLRKRKRKRISL

SEQ ID NO: 342 - protein, 31 aa, synthetic construct
  REGION 1..31: note = cell penetrating peptide
PAIVIDIWESGNSYSLHPSLLRKRKRKRLFL

SEQ ID NO: 343 - protein, 31 aa, synthetic construct
  REGION 1..31: note = cell penetrating peptide
IPIVVIHTHDSSGDFYLHPSLRKRKRKRKYL

SEQ ID NO: 344 - protein, 31 aa, synthetic construct
  REGION 1..31: note = cell penetrating peptide
APSVVFHITDTSGDFYLHPSLRKRKRKRKYL

SEQ ID NO: 345 - protein, 31 aa, synthetic construct
  REGION 1..31: note = cell penetrating peptide
PASTVISVDDDFADFYLHPSLRKRKRKYRIY

SEQ ID NO: 346 - protein, 52 aa, synthetic construct
  REGION 1..52: note = cell penetrating peptide
SVFVFTFSMNGLFDLHPSLHPRKRKRRYNCCFADGFLDNEQTTPVPTPQSRL

SEQ ID NO: 347 - protein, 30 aa, synthetic construct
  REGION 1..30: note = cell penetrating peptide
PFVIVDSFISSDTFYLHPSLVRKRKRRDLV

SEQ ID NO: 348 - protein, 29 aa, synthetic construct
  REGION 1..29: note = cell penetrating peptide
QPPDGVLVDDNDYYLHPGLYSRKRKRRVL

SEQ ID NO: 349 - protein, 30 aa, synthetic construct
  REGION 1..30: note = cell penetrating peptide
TVPVLEYDFTSADFTLHPSLSRKRKRRPSF

SEQ ID NO: 350 - protein, 34 aa, synthetic construct
  REGION 1..34: note = cell penetrating peptide
DTPVITIDIWSSDFYLHPSLSRKRKRRRKFVFVY

SEQ ID NO: 351 - protein, 33 aa, synthetic construct
  REGION 1..33: note = cell penetrating peptide
IPTEPHKKGKPIHVHRKRRCIKRRGKRCVKYSL

SEQ ID NO: 352 - protein, 37 aa, synthetic construct
  REGION 1..37: note = cell penetrating peptide
PTGPVFINGSTFYLYPSWYFARKRRKHVPLFFTDVAA

SEQ ID NO: 353 - protein, 39 aa, synthetic construct
  REGION 1..39: note = cell penetrating peptide
PSFDSVMVLGWDFILHPSYMWRKRRKPVPYFFADVRVAA

SEQ ID NO: 354 - protein, 39 aa, synthetic construct
  REGION 1..39: note = cell penetrating peptide
PQGHITVLGGAFYWHPSWYTARKRRKLVPNFLADVSVAA

SEQ ID NO: 355 - protein, 30 aa, synthetic construct
  REGION 1..30: note = cell penetrating peptide
IPLIIIHLDNSTGDYDLHPSLRKRRKLVHI

SEQ ID NO: 356 - protein, 32 aa, synthetic construct
  REGION 1..32: note = cell penetrating peptide
PIPAVLFNVLSSDLLLDPSLLRKRRKKYGVFS

SEQ ID NO: 357 - protein, 39 aa, synthetic construct
  REGION 1..39: note = cell penetrating peptide
PTARIIVYGGDFYLHPSYFGIRKRRKRVHHSFADVFVAA

SEQ ID NO: 358 - protein, 31 aa, synthetic construct
  REGION 1..31: note = cell penetrating peptide
IPAISVLIRGTDYYLNPAYYFRKRRKRILAY

SEQ ID NO: 359 - protein, 37 aa, synthetic construct
  REGION 1..37: note = cell penetrating peptide
PTGPVFITASGFYLYPTWYFTRKRRKRVSLFFTDVAA

SEQ ID NO: 360 - protein, 39 aa, synthetic construct
  REGION 1..39: note = cell penetrating peptide
PQYTIIADAGDFYLHPSYYMLRKRRKRLPYFFSDVSLAA

SEQ ID NO: 361 - protein, 36 aa, synthetic construct
  REGION 1..36: note = cell penetrating peptide
IPAISVLIHGTDYYLHPAYYLRKRRKRILAHQYVAT

SEQ ID NO: 362 - protein, 37 aa, synthetic construct
  REGION 1..37: note = cell penetrating peptide
PTGPVFISGAAFYLYPTWYFARKRRKRVSLFFADVAA

SEQ ID NO: 363 - protein, 39 aa, synthetic construct
  REGION 1..39: note = cell penetrating peptide
TKHSIVILGGDYYLWPYTHLLRKRRKRIPYFFTDGIVAH

SEQ ID NO: 364 - protein, 39 aa, synthetic construct
  REGION 1..39: note = cell penetrating peptide
PQTPIAVNGGDFYLHPSYTYVRKRRKRFPYFLADGYVAA

SEQ ID NO: 365 - protein, 39 aa, synthetic construct
  REGION 1..39: note = cell penetrating peptide
TQHAIVIQGGDYYLWPYTYLLRKRRKRIPYFFADGFVAY

SEQ ID NO: 366 - protein, 39 aa, synthetic construct
  REGION 1..39: note = cell penetrating peptide
PTSHVVVYGGDFYLHPSYYTIRKRRKRVHRFLSDVLVAA

SEQ ID NO: 367 - protein, 39 aa, synthetic construct
  REGION 1..39: note = cell penetrating peptide
PSSHIVVYGGDFYLHPAYYPTRKRRKRMHRFLSDVLVAA

SEQ ID NO: 368 - protein, 37 aa, synthetic construct
  REGION 1..37: note = cell penetrating peptide
PITPIFISGSQFYLHPSLYLARKRRKRVSLFFADVAA

SEQ ID NO: 369 - protein, 39 aa, synthetic construct
  REGION 1..39: note = cell penetrating peptide
VVPSMSVTVFSGDYFLHPSYFRKRRKRVSYFFADGIVAA

SEQ ID NO: 370 - protein, 30 aa, synthetic construct
  REGION 1..30: note = cell penetrating peptide
PLIIIEVLDGSGDYFLHPSLFRKRRKRPFF

SEQ ID NO: 371 - protein, 30 aa, synthetic construct
  REGION 1..30: note = cell penetrating peptide
TPLVIIALNNSTGDFELHPSLRKRRKRAYV

SEQ ID NO: 372 - protein, 37 aa, synthetic construct
  REGION 1..37: note = cell penetrating peptide
PTGPVFITGSGFYLHPAWYFARKRRKRIPLFFSDVAA

SEQ ID NO: 373 - protein, 30 aa, synthetic construct
  REGION 1..30: note = cell penetrating peptide
PVVVIHTHDTSGDFYLHPSLSRKRRKRKYL

SEQ ID NO: 374 - protein, 29 aa, synthetic construct
  REGION 1..29: note = cell penetrating peptide
VWIHTHDATGDFYLHPSLTLRKRRKRKYL

SEQ ID NO: 375 - protein, 32 aa, synthetic construct
  REGION 1..32: note = cell penetrating peptide
IGPSYDVAATNSDFIYDPDLFRKRRKRKLSAF

SEQ ID NO: 376 - protein, 32 aa, synthetic construct
  REGION 1..32: note = cell penetrating peptide
LKPALGLYTLGEDFIFDPDLFRKRRKRKYSDV

SEQ ID NO: 377 - protein, 30 aa, synthetic construct
  REGION 1..30: note = cell penetrating peptide
VVIIHTHDTTGDFYLHPSLTLRKRRKRKYL

SEQ ID NO: 378 - protein, 40 aa, synthetic construct
  REGION 1..40: note = cell penetrating peptide
AGPSIDVGDVGIDYFLDPYLFRKRRKRKRFFSFADDHVDS

SEQ ID NO: 379 - protein, 32 aa, synthetic construct
  REGION 1..32: note = cell penetrating peptide
PTVDLSDFELSMTFDLHPSLLRKRRKRKRTFL

SEQ ID NO: 380 - protein, 34 aa, synthetic construct
  REGION 1..34: note = cell penetrating peptide
RPPALGGFPGTDYYLHPGLSHRKRRKRRPFWFLL

SEQ ID NO: 381 - protein, 30 aa, synthetic construct
  REGION 1..30: note = cell penetrating peptide
VEPSLIITDSHDFELHPALWPRKRRRLDLF

SEQ ID NO: 382 - protein, 30 aa, synthetic construct
  REGION 1..30: note = cell penetrating peptide
ILPQYVIETFHDFFLTPDLYPRKRRRIDFF

SEQ ID NO: 383 - protein, 30 aa, synthetic construct
  REGION 1..30: note = cell penetrating peptide
PWYSIIERNFADFVLDPAFIPRKRRRLEIL

SEQ ID NO: 384 - protein, 33 aa, synthetic construct
  REGION 1..33: note = cell penetrating peptide
PGVLISYDGSMDPSLYYLLFYRKRRRLHRLFYR

SEQ ID NO: 385 - protein, 33 aa, synthetic construct
  REGION 1..33: note = cell penetrating peptide
NPGIPIAYYDNLDPSLIWWYLRKRRRLQHLFYR

SEQ ID NO: 386 - protein, 40 aa, synthetic construct
  REGION 1..40: note = cell penetrating peptide
THYVYIDGGDYFLWPVTFPVSRKRRRKRLSYFLADGFVAL

SEQ ID NO: 387 - protein, 40 aa, synthetic construct
  REGION 1..40: note = cell penetrating peptide
THYVYIDGGNFYLWPVTFSVSRKRRRKRLSYFFADGTVAL

SEQ ID NO: 388 - protein, 32 aa, synthetic construct
  REGION 1..32: note = cell penetrating peptide
IPSILIDGFSSDDFVLHPSHSRKRRRKRTPLL

SEQ ID NO: 389 - protein, 31 aa, synthetic construct
  REGION 1..31: note = cell penetrating peptide
IPVVVIHTHDYSGDFYLHPSLRKRRRKRKYL

SEQ ID NO: 390 - protein, 31 aa, synthetic construct
  REGION 1..31: note = cell penetrating peptide
TVVIHISDTSGDYYLHPSLQTRKRRRKRKYL

SEQ ID NO: 391 - protein, 33 aa, synthetic construct
  REGION 1..33: note = cell penetrating peptide
PPTVVVGSFGGVDYSLHPSLLRKRRRRRKRYIS

SEQ ID NO: 392 - protein, 33 aa, synthetic construct
  REGION 1..33: note = cell penetrating peptide
LVIADITMGEGTDYYLHPSLTRKRRRRRRRRSL

SEQ ID NO: 393 - protein, 30 aa, synthetic construct
  REGION 1..30: note = cell penetrating peptide
PTVVIRTSDSSGDFYLHPSLLRLKRKRKYL

SEQ ID NO: 394 - protein, 33 aa, synthetic construct
  REGION 1..33: note = cell penetrating peptide
EPLPPAIVELDNFDYDLHPSLRLKRKRKRTDLV

SEQ ID NO: 395 - protein, 36 aa, synthetic construct
  REGION 1..36: note = cell penetrating peptide
NDTPAVYIDPFSADYYLHPSLRLKRRRRRGQFVYVY

SEQ ID NO: 396 - protein, 32 aa, synthetic construct
  REGION 1..32: note = cell penetrating peptide
TPEIIVTFLDSSYTVADPSLFRLRKRRKRRFH

SEQ ID NO: 397 - protein, 30 aa, synthetic construct
  REGION 1..30: note = cell penetrating peptide
PFVVLHFSGGGTSFFLHPSLLRLRRKRVFY

SEQ ID NO: 398 - protein, 40 aa, synthetic construct
  REGION 1..40: note = cell penetrating peptide
IMFPQASHISSFDFLLHPSALRLRRKRKRSVHDDDGTIIE

SEQ ID NO: 399 - protein, 32 aa, synthetic construct
  REGION 1..32: note = cell penetrating peptide
PLPPTFVDFDNFDYDIHPGLLRRKKRKRTDLV

SEQ ID NO: 400 - protein, 31 aa, synthetic construct
  REGION 1..31: note = cell penetrating peptide
KPTVTFDALDIGGGFYLHPYIRRKKRKRMYL

SEQ ID NO: 401 - protein, 32 aa, synthetic construct
  REGION 1..32: note = cell penetrating peptide
LQPVIVVSPFESFDYNLHPSLRRKKRKRPYFF

SEQ ID NO: 402 - protein, 31 aa, synthetic construct
  REGION 1..31: note = cell penetrating peptide
GPAIIQSPTHSSFDYYLHPSLRRKKRKRKYL

SEQ ID NO: 403 - protein, 33 aa, synthetic construct
  REGION 1..33: note = cell penetrating peptide
LTPSFSIDVNYSDYNIHPANIRRKRKHSSSLYF

SEQ ID NO: 404 - protein, 43 aa, synthetic construct
  REGION 1..43: note = cell penetrating peptide
PYIVVDLYSGSMDYDIHPSLLRRKRKKRKRVYFSDGRVASRPK

SEQ ID NO: 405 - protein, 33 aa, synthetic construct
  REGION 1..33: note = cell penetrating peptide
SNASDTTSDFISVTFDLHPSLRRKRKKRKRRYI

SEQ ID NO: 406 - protein, 39 aa, synthetic construct
  REGION 1..39: note = cell penetrating peptide
PHGHVVVFGGDFLWHPSWYSPRRKRKRLPTFFADVSVAA

SEQ ID NO: 407 - protein, 39 aa, synthetic construct
  REGION 1..39: note = cell penetrating peptide
PQGHVYVSGGDFLWHPSLYTPRRKRKRVHTFFADVSVAA

SEQ ID NO: 408 - protein, 39 aa, synthetic construct
  REGION 1..39: note = cell penetrating peptide
PSAHIILYGGDFYLHPSYLGIRRKRKRMHNFFSDVYVAA

SEQ ID NO: 409 - protein, 30 aa, synthetic construct
  REGION 1..30: note = cell penetrating peptide
PAVVIHFGEPGGDFYLHPDLQRRKRKRAYL

SEQ ID NO: 410 - protein, 30 aa, synthetic construct
  REGION 1..30: note = cell penetrating peptide
DGNIIIYSTYFKHYYLHPSLYRRKRKRLLD

SEQ ID NO: 411 - protein, 32 aa, synthetic construct
  REGION 1..32: note = cell penetrating peptide
DGSIALSLEYFRHYYLHPSLLRRKRKRNPIFI

SEQ ID NO: 412 - protein, 30 aa, synthetic construct
  REGION 1..30: note = cell penetrating peptide
DGDISLSVEYFRHYYLHPSLLRRKRKRLFN

SEQ ID NO: 413 - protein, 40 aa, synthetic construct
  REGION 1..40: note = cell penetrating peptide
PGTPEVVIDIYPHTPLAFLHHRRKRKRGSSVFFADVLLAF

SEQ ID NO: 414 - protein, 31 aa, synthetic construct
  REGION 1..31: note = cell penetrating peptide
PSILINESSSDDFVLHPSHIPRRKRKRAYPF

SEQ ID NO: 415 - protein, 30 aa, synthetic construct
  REGION 1..30: note = cell penetrating peptide
VVPGFDINVEASDFNIHPSVLRRKRKRSMF

SEQ ID NO: 416 - protein, 30 aa, synthetic construct
  REGION 1..30: note = cell penetrating peptide
PSILLFYPDSSPSFYLHPSLLRRKRKRVFY

SEQ ID NO: 417 - protein, 31 aa, synthetic construct
  REGION 1..31: note = cell penetrating peptide
PSILIDEFSSDDFVLHPSHIPRRKRKRIDSL

SEQ ID NO: 418 - protein, 30 aa, synthetic construct
  REGION 1..30: note = cell penetrating peptide
PAVVIHFGESGADYYLHPHLQRRKRKRAYL

SEQ ID NO: 419 - protein, 30 aa, synthetic construct
  REGION 1..30: note = cell penetrating peptide
DGNIIIYSTYLRHYYLHPSLYRRKRKRLLD

SEQ ID NO: 420 - protein, 30 aa, synthetic construct
  REGION 1..30: note = cell penetrating peptide
PVVIIHPHDSTGDFYLHPSLHRRKRKRKYL

SEQ ID NO: 421 - protein, 32 aa, synthetic construct
  REGION 1..32: note = cell penetrating peptide
DDPDITLAIFGTDFYLHPGLLRRKRKRKNFSV

SEQ ID NO: 422 - protein, 32 aa, synthetic construct
  REGION 1..32: note = cell penetrating peptide
EPPIVVDGFDAFDTFYLHPSHRRKRKRKRSGF

SEQ ID NO: 423 - protein, 31 aa, synthetic construct
  REGION 1..31: note = cell penetrating peptide
PSILIDGFSSDDFVLHPSHIPRRKRKRKRSL

SEQ ID NO: 424 - protein, 30 aa, synthetic construct
  REGION 1..30: note = cell penetrating peptide
DDTLDTFQNYNDYDLHPSLLPRRKRKRRIL

SEQ ID NO: 425 - protein, 49 aa, synthetic construct
  REGION 1..49: note = cell penetrating peptide
DVIIEYPEAGGSYFLHPTAPCRRKRRYCFADGLLDAGQSEVLSPPCACY

SEQ ID NO: 426 - protein, 38 aa, synthetic construct
  REGION 1..38: note = cell penetrating peptide
TTPHAGVYVSVDFWLHPGLLSRRKRRFPFLFTDGIVAA

SEQ ID NO: 427 - protein, 33 aa, synthetic construct
  REGION 1..33: note = cell penetrating peptide
PPYIIDFNDNSATFYLHPSLIRRKRRKRKRIFS

SEQ ID NO: 428 - protein, 31 aa, synthetic construct
  REGION 1..31: note = cell penetrating peptide
PVIIDFGTSGATFYLHPSLFLRRKRRKRRFL

SEQ ID NO: 429 - protein, 32 aa, synthetic construct
  REGION 1..32: note = cell penetrating peptide
HPPIYVGSVPGVDYYLHPSLSRRKRRRRHSYL

SEQ ID NO: 430 - protein, 34 aa, synthetic construct
  REGION 1..34: note = cell penetrating peptide
PIIIVDVAVGAGTDYYLHPSLRRKRRRRGRRHFM

SEQ ID NO: 431 - protein, 33 aa, synthetic construct
  REGION 1..33: note = cell penetrating peptide
GIIIDLSEELFKHYYLHESLLRRKRRRRKRLYA

SEQ ID NO: 432 - protein, 35 aa, synthetic construct
  REGION 1..35: note = cell penetrating peptide
SDTPIVTLDIWSSDFYLHPSLRRKRRRRRKYVFVY

SEQ ID NO: 433 - protein, 35 aa, synthetic construct
  REGION 1..35: note = cell penetrating peptide
PRPDVVIYYYGGVDYSLHPSLRRKRRRRRRKRVSF

SEQ ID NO: 434 - protein, 31 aa, synthetic construct
  REGION 1..31: note = cell penetrating peptide
PIVVIHPHDNSGDFYLHPSLTRRLRRKRKYL

SEQ ID NO: 435 - protein, 33 aa, synthetic construct
  REGION 1..33: note = cell penetrating peptide
PDIILDIYTPGSTFYLHPSHYRRRGRKRKRTVF

SEQ ID NO: 436 - protein, 31 aa, synthetic construct
  REGION 1..31: note = cell penetrating peptide
PLVIIDVLGSGDFLLHPSLLQRRRKRIKSVF

SEQ ID NO: 437 - protein, 31 aa, synthetic construct
  REGION 1..31: note = cell penetrating peptide
PGTEIVFIPEDSIDFYLHPSLRRRKRKYFVR

SEQ ID NO: 438 - protein, 31 aa, synthetic construct
  REGION 1..31: note = cell penetrating peptide
LPTVIIHTADTSGDFYLHPSLRRRKRKRTYL

SEQ ID NO: 439 - protein, 31 aa, synthetic construct
  REGION 1..31: note = cell penetrating peptide
LPAVVIHTSDTSGDFYLHPSLRRRKRKRAYL

SEQ ID NO: 440 - protein, 31 aa, synthetic construct; identical to SEQ ID NO: 439
  REGION 1..31: note = cell penetrating peptide

SEQ ID NO: 441 - protein, 31 aa, synthetic construct
  REGION 1..31: note = cell penetrating peptide
TPTIIIHTEDFSGDYYLHPSLRRRKRKRAYL

SEQ ID NO: 442 - protein, 41 aa, synthetic construct
  REGION 1..41: note = cell penetrating peptide
RPGVGLVDNAFMFAWGPQYILRRRKRKRPLPPLFADGFVAA

SEQ ID NO: 443 - protein, 32 aa, synthetic construct
  REGION 1..32: note = cell penetrating peptide
IIQPAMAVDVYDDFYLHPHLLRRRKRKRLDFF

SEQ ID NO: 444 - protein, 31 aa, synthetic construct
  REGION 1..31: note = cell penetrating peptide
PIGPSAALDVEYYDFDLHPSLRRRKRKRNMF

SEQ ID NO: 445 - protein, 32 aa, synthetic construct
  REGION 1..32: note = cell penetrating peptide
TPSILIDEFSSDDFILHPSHIRRRKRKRLNSL

SEQ ID NO: 446 - protein, 31 aa, synthetic construct
  REGION 1..31: note = cell penetrating peptide
PLQPPYEAEVVFDTFDLHPSLRRRKRKRSSF

SEQ ID NO: 447 - protein, 32 aa, synthetic construct
  REGION 1..32: note = cell penetrating peptide
PYAPPLLYDFESHDFILHPSHRRRKRKRIAMF

SEQ ID NO: 448 - protein, 31 aa, synthetic construct
  REGION 1..31: note = cell penetrating peptide
GVDINVGSIYPSVDYYLHPSLRRRKRKRTLH

SEQ ID NO: 449 - protein, 31 aa, synthetic construct
  REGION 1..31: note = cell penetrating peptide
GTTHIVGSEFSSVDYYLHPSLRRRKRKRNFH

SEQ ID NO: 450 - protein, 31 aa, synthetic construct
  REGION 1..31: note = cell penetrating peptide
LPVVIIHTHDNSGDFFLHPSLRRRKRKRKYL

SEQ ID NO: 451 - protein, 31 aa, synthetic construct
  REGION 1..31: note = cell penetrating peptide
IPVVVIHTHDNSGDFYLHPSLRRRKRKRKYL

SEQ ID NO: 452 - protein, 31 aa, synthetic construct
  REGION 1..31: note = cell penetrating peptide
LPVVVIHTHDNSGDFYLHPSLRRRKRKRKYL

SEQ ID NO: 453 - protein, 31 aa, synthetic construct
  REGION 1..31: note = cell penetrating peptide
GPAIVQSLTHTSLDYYLHPSLRRRKRKRKYL

SEQ ID NO: 454 - protein, 31 aa, synthetic construct
  REGION 1..31: note = cell penetrating peptide
IIILVDSPDTSGVFDLHPSLLRRRKRRYMWN

SEQ ID NO: 455 - protein, 37 aa, synthetic construct
  REGION 1..37: note = cell penetrating peptide
VPPLPGYSNYGGPDFYLHPSLRRRKRRKRRYLSSFVF

SEQ ID NO: 456 - protein, 39 aa, synthetic construct
  REGION 1..39: note = cell penetrating peptide
RPHVIYRGYNGTDYYLHPSLSRRRRNSRHIYFSDGVLAA

SEQ ID NO: 457 - protein, 36 aa, synthetic construct
  REGION 1..36: note = cell penetrating peptide
PGVLVGYNDFAKDPSLYWWFIRRRRARRFHPYSRSR

SEQ ID NO: 458 - protein, 39 aa, synthetic construct
  REGION 1..39: note = cell penetrating peptide
PQGNFVMVSGWDFILHPSYFWRRRRKPVPYFFADVRVAA

SEQ ID NO: 459 - protein, 39 aa, synthetic construct
  REGION 1..39: note = cell penetrating peptide
PSSHVVVYGGDFYLHPSYYPVRRRRKHMPRFLSDVVVAA

SEQ ID NO: 460 - protein, 30 aa, synthetic construct
  REGION 1..30: note = cell penetrating peptide
MIPWIIIGTQGSDYDLHPSLLRRRRKLSFL

SEQ ID NO: 461 - protein, 37 aa, synthetic construct
  REGION 1..37: note = cell penetrating peptide
PTGPVFITGSDFYLHPTWYFARRRRKRIPLFFTDVAA

SEQ ID NO: 462 - protein, 30 aa, synthetic construct
  REGION 1..30: note = cell penetrating peptide
DGNIVIYSTYLKHYYLHPSLYRRRRKRLLD

SEQ ID NO: 463 - protein, 39 aa, synthetic construct
  REGION 1..39: note = cell penetrating peptide
PFDTIVVDGADFVLHPSYFILRRRRKRFPYFFTDVRVAA

SEQ ID NO: 464 - protein, 39 aa, synthetic construct
  REGION 1..39: note = cell penetrating peptide
PSTSIIVDGTDFILHPSYFLLRRRRKRFPYFFTDVRVAA

SEQ ID NO: 465 - protein, 39 aa, synthetic construct
  REGION 1..39: note = cell penetrating peptide
PFNTIIVDGADFMLHPSYFILRRRRKRFPYFFADVRVAA

SEQ ID NO: 466 - protein, 39 aa, synthetic construct
  REGION 1..39: note = cell penetrating peptide
SPFNNVLVYGSDFILHPSYFLRRRRKRFPYFFADVRVAA

SEQ ID NO: 467 - protein, 39 aa, synthetic construct
  REGION 1..39: note = cell penetrating peptide
HYVYVDGGDFYLWPITFYVSHRRRRKRVSYFFTDGTVAT

SEQ ID NO: 468 - protein, 39 aa, synthetic construct
  REGION 1..39: note = cell penetrating peptide
QYVYIDGGDFYLWPVTFSLFGRRRRKRLSYFFADGTVAL

SEQ ID NO: 469 - protein, 39 aa, synthetic construct
  REGION 1..39: note = cell penetrating peptide
PSADIIVNGGDFYLHPSYLSLRRRRKRMHRFFADVLVAA

SEQ ID NO: 470 - protein, 39 aa, synthetic construct
  REGION 1..39: note = cell penetrating peptide
PTAHIVVHGGDFYLHPSYLYVRRRRKRLHHFFTDVYVAA

SEQ ID NO: 471 - protein, 39 aa, synthetic construct
  REGION 1..39: note = cell penetrating peptide
GGDREVVVNMYPYSIMSFLHYRRRRKRGYVYFSDVILAI

SEQ ID NO: 472 - protein, 40 aa, synthetic construct
  REGION 1..40: note = cell penetrating peptide
PEFPEIVIDGYGLSALTFVLHRRRRKRGSHLYFSDVLLAV

SEQ ID NO: 473 - protein, 30 aa, synthetic construct
  REGION 1..30: note = cell penetrating peptide
PAIIIDFTDSTATFYLHPSLMRRRRKRHIF

SEQ ID NO: 474 - protein, 30 aa, synthetic construct
  REGION 1..30: note = cell penetrating peptide
AVVIHTEDSSGDFYLHPSLLQRRRRKRKYL

SEQ ID NO: 475 - protein, 30 aa, synthetic construct
  REGION 1..30: note = cell penetrating peptide
VVIIHTYDTSGDFYLHPSLTTRRRRKRKYL

SEQ ID NO: 476 - protein, 30 aa, synthetic construct
  REGION 1..30: note = cell penetrating peptide
VVIHVEDSSGDYYLHPSLRVTRRRRKRKYL

SEQ ID NO: 477 - protein, 33 aa, synthetic construct
  REGION 1..33: note = cell penetrating peptide
DAPLVTINALGSDYDLHPSYLRRRRKRKYSEIF

SEQ ID NO: 478 - protein, 31 aa, synthetic construct
  REGION 1..31: note = cell penetrating peptide
GTITPSFSFNNSGDFVLHPSLRRRRKRKFVF

SEQ ID NO: 479 - protein, 31 aa, synthetic construct
  REGION 1..31: note = cell penetrating peptide
DIIIHDVGSSVDYILHPGLLPRRRRKRKRSY

SEQ ID NO: 480 - protein, 32 aa, synthetic construct
  REGION 1..32: note = cell penetrating peptide
SEPDIRLDWFSPDYDLHPSLLRRRRKRKRNMF

SEQ ID NO: 481 - protein, 46 aa, synthetic construct
  REGION 1..46: note = cell penetrating peptide
PLEPPIILDFQSSTYYLHPSLRRRRKRKRSDIYDFLSDGSVDTPEW

SEQ ID NO: 482 - protein, 33 aa, synthetic construct
  REGION 1..33: note = cell penetrating peptide
GPDLIINLYGNDYSLHPSYLLRRRRKRKRFHVL

SEQ ID NO: 483 - protein, 32 aa, synthetic construct
  REGION 1..32: note = cell penetrating peptide
DAPDLTSSEYLSVTFDLHPSLRRRRKRKRRYI

SEQ ID NO: 484 - protein, 30 aa, synthetic construct
  REGION 1..30: note = cell penetrating peptide
PSVVILSTDDFGDMFRFWHLLRRRRKRRYL

SEQ ID NO: 485 - protein, 30 aa, synthetic construct
  REGION 1..30: note = cell penetrating peptide
PDVVIYSTDDFGYMFRFWHFLRRRRKRRYL

SEQ ID NO: 486 - protein, 30 aa, synthetic construct
  REGION 1..30: note = cell penetrating peptide
PHVVIYYTDDYGDMFRFWHLLRRRRKRRYL

SEQ ID NO: 487 - protein, 30 aa, synthetic construct
  REGION 1..30: note = cell penetrating peptide
PHVNIYYTDEFGDMFRFWHFLRRRRKRRYL

SEQ ID NO: 488 - protein, 35 aa, synthetic construct
  REGION 1..35: note = cell penetrating peptide
RPAPSDDTVYYSAGVLDPSLLRRRRKRRRSVAYGF

SEQ ID NO: 489 - protein, 40 aa, synthetic construct
  REGION 1..40: note = cell penetrating peptide
DTTHDVVIHGSTFALWPVYFLRRRRRKHVPYFLADGGVAA

SEQ ID NO: 490 - protein, 40 aa, synthetic construct
  REGION 1..40: note = cell penetrating peptide
THYVYIDGGDFYLWPVTFFLPRRRRRKRVSYFLADGTVAL

SEQ ID NO: 491 - protein, 40 aa, synthetic construct
  REGION 1..40: note = cell penetrating peptide
THYVYIDGGDFYLWPVTLFVPRRRRRKRLSYFLADGTVAL

SEQ ID NO: 492 - protein, 40 aa, synthetic construct
  REGION 1..40: note = cell penetrating peptide
DVTHDVYIQGSSFALWPVYFFRRRRRKRIPYFFADGDVAA

SEQ ID NO: 493 - protein, 29 aa, synthetic construct
  REGION 1..29: note = cell penetrating peptide
THYVYIDGGDFYLWPVTFYLSRRRRRKRV

SEQ ID NO: 494 - protein, 40 aa, synthetic construct
  REGION 1..40: note = cell penetrating peptide
THYVYINGGDFYLWPVTFPLSRRRRRKRVSYFFTDGTLAP

SEQ ID NO: 495 - protein, 40 aa, synthetic construct
  REGION 1..40: note = cell penetrating peptide
THYVYIDGADFYLWPVALFVPRRRRRKRISYFLADGTVAL

SEQ ID NO: 496 - protein, 31 aa, synthetic construct
  REGION 1..31: note = cell penetrating peptide
PAVVIHTYDNSGDFYLHPSLLRRRRRKRKYL

SEQ ID NO: 497 - protein, 31 aa, synthetic construct
  REGION 1..31: note = cell penetrating peptide
PVVVIHTHDNSGDFYLHPSLFRRRRRKRKYL

SEQ ID NO: 498 - protein, 33 aa, synthetic construct
  REGION 1..33: note = cell penetrating peptide
PRVVVFYYGGVDYSLHPSLLFRRRRRKRRRHAF

SEQ ID NO: 499 - protein, 33 aa, synthetic construct
  REGION 1..33: note = cell penetrating peptide
FPSGVDINVTDPSLYWLRWWLRRRRRRGYLLFR

SEQ ID NO: 500 - protein, 32 aa, synthetic construct
  REGION 1..32: note = cell penetrating peptide
PTVVVGYFGGVDYSLHPSLMFRRRRRRKRYIS

SEQ ID NO: 501 - protein, 30 aa, synthetic construct
  REGION 1..30: note = cell penetrating peptide
SDVVIYSTDDFGDMFRFWHFLRRRRRRRYL

SEQ ID NO: 502 - protein, 32 aa, synthetic construct
  REGION 1..32: note = cell penetrating peptide
IIVDVASGAGTDYYLHPSLQARRRRRRRRHFM

SEQ ID NO: 503 - protein, 38 aa, synthetic construct
  REGION 1..38: note = cell penetrating peptide
APVSYGGLVSMDPNSLFWFLLRRRRRRRRTTKRILLNR

SEQ ID NO: 504 - protein, 32 aa, synthetic construct
  REGION 1..32: note = cell penetrating peptide
IIVDVERGSGSDYYLHPSLTLRRRRRRRRKSL

SEQ ID NO: 505 - protein, 33 aa, synthetic construct
  REGION 1..33: note = cell penetrating peptide
IVDVARGPSSDYFLHPSLYATRRRRRRRRRKHI

SEQ ID NO: 506 - protein, 33 aa, synthetic construct
  REGION 1..33: note = cell penetrating peptide
IIIVDVIGASGSDYYLHPALSRRRRRRRRRRSL

SEQ ID NO: 507 - protein, 33 aa, synthetic construct
  REGION 1..33: note = cell penetrating peptide
VIIVDVSVGSSTDYYLHPSLARRRRRRRRRRSL

SEQ ID NO: 508 - protein, 33 aa, synthetic construct
  REGION 1..33: note = cell penetrating peptide
IIIVDIIGGAGSDYYLHPSLSRRRRRRRRRRSQ

SEQ ID NO: 509 - protein, 31 aa, synthetic construct
  REGION 1..31: note = cell penetrating peptide
APAVVIHVEDSSGDFYLHPSLRSKKRKRKYL

SEQ ID NO: 510 - protein, 32 aa, synthetic construct
  REGION 1..32: note = cell penetrating peptide
PSSLQPTNYFDYWQLFEPSLWRSRKRKRNVYY

SEQ ID NO: 511 - protein, 31 aa, synthetic construct
  REGION 1..31: note = cell penetrating peptide
IPAVIVHIIDTSFDYYLHPSLRTKRRKRKYL

SEQ ID NO: 512 - protein, 39 aa, synthetic construct
  REGION 1..39: note = cell penetrating peptide
PYWPGEGWPPPHFEFSDPSLYRVKKRRWDDCIAIMITVT

SEQ ID NO: 513 - protein, 31 aa, synthetic construct
  REGION 1..31: note = cell penetrating peptide
VPAVIVHITDTSGDFYLHPSLRWKKRKRKYL

SEQ ID NO: 514 - protein, 31 aa, synthetic construct
  REGION 1..31: note = cell penetrating peptide
TPVVVIHTEDNTGDFYLHPSLRWRKRKRHYL

SEQ ID NO: 515 - protein, 31 aa, synthetic construct
  REGION 1..31: note = cell penetrating peptide
IPVVVIHTHDNTGDFYLHPSLRWRKRKRKYL

SEQ ID NO: 516 - protein, 33 aa, synthetic construct
  REGION 1..33: note = cell penetrating peptide
GQPSLLWDPSTGTFDLHPGLLRWRRKRRRRHDL

SEQ ID NO: 517 - protein, 31 aa, synthetic construct
  REGION 1..31: note = cell penetrating peptide
IPTVVIHVADSSGDFYLHPSLRWRRRKRKYL

SEQ ID NO: 518 - protein, 473 aa, synthetic construct
  REGION 1..473: note = minor capsid protein of a papillomavirus
MRHKRSAKRTKRASATQLYKTCKQAGTCPPDIIPKVEGKTIAEQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 519 - protein, 10 aa, synthetic construct
  REGION 1..10: note = cell penetrating peptide
RKKRRRQRRR

SEQ ID NO: 520 - protein, 11 aa, synthetic construct
  REGION 1..11: note = cell penetrating peptide
PLSSIFSRIDG

SEQ ID NO: 521 - protein, 12 aa, synthetic construct
  REGION 1..12: note = cell penetrating peptide
AAVLLPVLLAAP

SEQ ID NO: 522 - protein, 10 aa, synthetic construct
  REGION 1..10: note = nuclear export signal (NES) sequence
LPPLERLTLD

SEQ ID NO: 523 - protein, 16 aa, synthetic construct
  REGION 1..16: note = GFP11
RDHMVLHEYVNAAGIT

SEQ ID NO: 524 - protein, 5 aa, synthetic construct
  REGION 1..5: note = linker sequence
GGSGG

SEQ ID NO: 525 - protein, 171 aa, synthetic construct
  REGION 1..171: note = GFP11-CD8-CIMPR
MALPVTALLLPLALLLHAARPRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGKSQFRV

SEQ ID NO: 526 - protein, 161 aa, synthetic construct
  REGION 1..161: note = CD8-CIMPR-GFP11
DDSDEDLLHVGSSGSSRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGK

SEQ ID NO: 527 - protein, 174 aa, synthetic construct
  REGION 1..174: note = L2-GFP11-CPP
FYLHPSYYMLRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGKFRKRRKRLPYFFSDVSLAA

SEQ ID NO: 528 - protein, 180 aa, synthetic construct
  REGION 1..180: note = L2-CPP-GFP11
YYMLRKRRKRLPYFFSDVSLAADASGSSGSSRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGSGGRDHMVLHEYVNAAGITGGKFPRL

SEQ ID NO: 529 - protein, 6 aa, synthetic construct
  REGION 1..6: note = synthetic peptide
RKRRKR

SEQ ID NO: 530 - protein, 27 aa, synthetic construct
  REGION 1..27: note = synthetic peptide
PQYTIIADAGDFYLHPSYYMLRKRRKR

SEQ ID NO: 531 - protein, 39 aa, synthetic construct; identical to SEQ ID NO: 360
  REGION 1..39: note = synthetic peptide

SEQ ID NO: 532 - protein, 31 aa, synthetic construct
  REGION 1..31: note = synthetic peptide
IPVVIIHPHDSTGDFYLHPSLHRRKRKRKYL

SEQ ID NO: 533 - protein, 28 aa, synthetic construct
  REGION 1..28: note = synthetic peptide
SPQYTIIADAGDFYLHPSYYMLRKRRKR

SEQ ID NO: 534 - DNA, 19 bp, synthetic construct
  misc_feature 1..19: note = primer
  source 1..19: mol_type = other DNA
gcacccagagcatgagaat

SEQ ID NO: 535 - DNA, 19 bp, synthetic construct
  misc_feature 1..19: note = primer
  source 1..19: mol_type = other DNA
tcgtaggtggtggttctct

SEQ ID NO: 536 - DNA, 20 bp, synthetic construct
  misc_feature 1..20: note = primer
  source 1..20: mol_type = other DNA
aatgtttcaggacccacagg

SEQ ID NO: 537 - DNA, 21 bp, synthetic construct
  misc_feature 1..21: note = primer
  source 1..21: mol_type = other DNA
ctcacgtcgcagtaactgttg

SEQ ID NO: 538 - DNA, 22 bp, synthetic construct
  misc_feature 1..22: note = primer
  source 1..22: mol_type = other DNA
ctgctgtcaccttcaccgttcc

SEQ ID NO: 539 - DNA, 20 bp, synthetic construct
  misc_feature 1..20: note = primer
  source 1..20: mol_type = other DNA
agtactccgtgtggatcggc

SEQ ID NO: 540 - protein, 33 aa, synthetic construct
  REGION 1..33: note = C-terminus of the L2 protein of HPV
ADAGDFYLHPSYYMLRKRRKRLPYFFSDVSLAA

SEQ ID NO: 541 - protein, 22 aa, synthetic construct
  REGION 1..22: note = C-terminus of the L2 protein of HPV
DSTGDFYLHPSLHRRKRKRKYL

SEQ ID NO: 542 - protein, 31 aa, synthetic construct
  REGION 1..31: note = C-terminus of the L2 protein of HPV
ITGSGFYLHPAWYFARKRRKRIPLFFSDVAA

SEQ ID NO: 543 - protein, 33 aa, synthetic construct
  REGION 1..33: note = C-terminus of the L2 protein of HPV
IHGTHYYLWPLYYFIPKKRKRVPYFFADGFVAA

SEQ ID NO: 544 - protein, 33 aa, synthetic construct
  REGION 1..33: note = C-terminus of the L2 protein of HPV
VDGADFVLHPSYFILRRRRKRFPYFFTDVRVAA

SEQ ID NO: 545 - protein, 33 aa, synthetic construct; identical to SEQ ID NO: 540
  REGION 1..33: note = C-terminus of the L2 protein of HPV

SEQ ID NO: 546 - protein, 33 aa, synthetic construct
  REGION 1..33: note = C-terminus of the L2 protein of HPV
ADAGDFYLHPSYYMLAAAAAALPYFFSDVSLAA

SEQ ID NO: 547 - protein, 36 aa, synthetic construct
  REGION 1..36: note = C-terminus of the L2 protein of HPV
ADAGDFYLHPSYYMLRKKRRQRRRLPYFFSDVSLAA

SEQ ID NO: 548 - protein, 39 aa, synthetic construct
  REGION 1..39: note = C-terminus of the L2 protein of HPV
ADAGDFYLHPSYYMLPLSSIFSRIGDPLPYFFSDVSLAA

SEQ ID NO: 549 - protein, 39 aa, synthetic construct
  REGION 1..39: note = C-terminus of the L2 protein of HPV
ADAGDFYLHPSYYMLAAVLLPVLLAAPLPYFFSDVSLAA

SEQ ID NO: 550 - protein, 30 aa, synthetic construct
  REGION 1..30: note = C-terminus of the L2 protein of HPV
ADAGDFYLHPSYYMLRRRLPYFFSDVSLAA

SEQ ID NO: 551 - protein, 31 aa, synthetic construct
  REGION 1..31: note = C-terminus of the L2 protein of HPV
ADAGDFYLHPSYYMLRRRRLPYFFSDVSLAA

SEQ ID NO: 552 - protein, 32 aa, synthetic construct
  REGION 1..32: note = C-terminus of the L2 protein of HPV
ADAGDFYLHPSYYMLRRRRRLPYFFSDVSLAA

SEQ ID NO: 553 - protein, 33 aa, synthetic construct
  REGION 1..33: note = C-terminus of the L2 protein of HPV
ADAGDFYLHPSYYMLRRRRRRLPYFFSDVSLAA

SEQ ID NO: 554 - protein, 34 aa, synthetic construct
  REGION 1..34: note = C-terminus of the L2 protein of HPV
ADAGDFYLHPSYYMLRRRRRRRLPYFFSDVSLAA

SEQ ID NO: 555 - protein, 33 aa, synthetic construct
  REGION 1..33: note = C-terminus of the L2 protein of HPV
ADAGDFYLHPSYYMLKKKKKKLPYFFSDVSLAA

SEQ ID NO: 556 - protein, 28 aa, synthetic construct
  REGION 1..28: note = synthetic peptide
SPQYTIIADAGDFYLHPSYYMLAAAAAA

SEQ ID NO: 557 - protein, 25 aa, synthetic construct
  REGION 1..25: note = synthetic peptide
SPQYTIIADAGDFYLHPSYYMLRRR

SEQ ID NO: 558 - protein, 27 aa, synthetic construct
  REGION 1..27: note = synthetic peptide
PQYTIIADAGDFYLHPSYYMLAAAAAA

SEQ ID NO: 559 - protein, 24 aa, synthetic construct
  REGION 1..24: note = synthetic peptide
PQYTIIADAGDFYLHPSYYMLRRR

SEQ ID NO: 560 - protein, 29 aa, synthetic construct
  REGION 1..29: note = synthetic peptide
CSPQYTIIADAGDFYLHPSYYMLRKRRKR

SEQ ID NO: 561 - protein, 26 aa, synthetic construct
  REGION 1..26: note = synthetic peptide
CSPQYTIIADAGDFYLHPSYYMLRRR

SEQ ID NO: 562 - protein, 29 aa, synthetic construct
  REGION 1..29: note = synthetic peptide
CSPQYTIIADAGDAAAHPSAAAARKRRKR

SEQ ID NO: 563 - protein, 32 aa, synthetic construct
  REGION 1..32: note = synthetic peptide
CSPQYTIIADAGDFYLHPSYYMLRRQRKKRRR

SEQ ID NO: 564 - protein, 29 aa, synthetic construct
  REGION 1..29: note = synthetic peptide
CSPQYTIIADAGDFYLHPSYYMLKRRRKR

SEQ ID NO: 565 - protein, 29 aa, synthetic construct
  REGION 1..29: note = synthetic peptide
CSPQYTIIADAGDFYLHPSYYMLAAAAAA

SEQ ID NO: 566 - protein, 26 aa, synthetic construct
  REGION 1..26: note = synthetic peptide
CSPQYTIIADAGDFYLHPSYYMLRRR

SEQ ID NO: 567 - protein, 38 aa, synthetic construct
  REGION 1..38: note = synthetic peptide
QYTIIADAGDFYLHPSYYMLRKRRKRLPYFFSDVSLAA

SEQ ID NO: 568 - protein, 27 aa, synthetic construct
  REGION 1..27: note = synthetic peptide
IIHPHDSTGDFYLHPSLHRRKRKRKYL

SEQ ID NO: 569 - protein, 24 aa, synthetic construct
  REGION 1..24: note = synthetic peptide
HLGLTAQPELYLLNTMDADSLVSR